(19) United States
(12) Patent Application Publication
(10) Pub. No.: US 2023/0149478 A1
(43) Pub. Date: May 18, 2023

(54) COMPOSITIONS AND METHODS FOR DIABETES TREATMENT

(71) Applicant: The General Hospital Corporation, Boston, MA (US)

(72) Inventor: Denise L. FAUSTMAN, Boston, MA (US)

(21) Appl. No.: 17/913,503

(22) PCT Filed: Mar. 25, 2021
(86) PCT No.: PCT/US2021/024101

§ 371 Date: Sep. 22, 2022

Related U.S. Application Data
(60) Provisional application No. 62/994,507, filed on Mar. 25, 2020.

(51) Int. Cl. A61K 35/741; G01N 33/50; A61P 3/10

(52) U.S. Cl. CPC A61K 35/741; G01N 33/5038; A61P 3/10; G01N 2800/52; G01N 2800/042

(57) ABSTRACT

Described are compositions and methods for determining the propensity of a subject with diabetes (e.g., type 1 diabetes) to benefit from treatment with Bacillus Calmette-Guerin (BCG). Using the compositions and methods of the disclosure, a subject with diabetes (e.g., type 1 diabetes) may be identified as likely to respond to BCG therapy on the basis, for example, of a determination that the subject was young (e.g., less than 40 years of age) at the time of onset of the diabetes. Additionally, or alternatively, the subject may be identified as likely to respond to BCG therapy based on a determination that the subject exhibits a reduced rate of glucose uptake, an elevated rate of oxidative phosphorylation, and/or a reduced rate of aerobic glycolysis, for example, as compared to a healthy subject (e.g., a subject without diabetes). The compositions and methods of the disclosure can additionally be used to administer BCG to subjects identified as likely to respond to treatment with a composition comprising BCG.

### BACKGROUND

Diabetes is a severe autoimmune disease that is characterized by insulin deficiency that prevents normal regulation of blood glucose levels. Insulin is a peptide hormone produced by p cells within the islets of Langerhans of the pancreas (β-islet cells). Insulin promotes glucose utilization, protein synthesis, formation and storage of neutral lipids, and is the primary source of energy for brain and muscle tissue. Type 1 diabetes is caused by an autoimmune reaction that results in destruction of the p-islet cells of the pancreas, which eliminates or reduces insulin production and eventually results in hyperglycemia and ketoacidosis. Insulin injection therapy has been used to treat severe hyperglycemia and ketoacidosis, but may be insufficient to normalize blood glucose levels. Insulin injection therapy may also be insufficient to prevent premature vascular deterioration, which is a leading cause of morbidity among diabetics today. Diabetes-related vascular deterioration, which includes both microvascular deterioration and acceleration of atherosclerosis, can result in renal failure, retinal deterioration, angina pectoris, myocardial infarction, peripheral neuropathy, and atherosclerosis.

There exists a need for therapies for the treatment of diabetes, such as type 1 diabetes, as well as compositions and methods for identifying subjects that are likely to benefit from such therapies.

### SUMMARY OF THE DISCLOSURE

The present disclosure features compositions and methods for treating a subject (e.g., a mammalian subject, such as a human subject) that has diabetes (e.g., type 1 diabetes) by administration of Bacillus Calmette-Guerin (BCG). The disclosure also features compositions and methods for determining the likelihood of a subject with diabetes (e.g., type 1 diabetes) to respond to treatment with BCG. Using the compositions and methods described herein, a subject with diabetes may be assessed for their propensity to benefit from BCG treatment. If the subject is identified as likely to respond to BCG therapy, the subject may be treated accordingly. The disclosure features methods for identifying a diabetes subject that is likely to benefit from BCG treatment so that they can be selected and treated accordingly.

The present disclosure is based, in part, on the discovery of characteristics that identify a subject as having a high likelihood of responding to BCG treatment. For example, a subject with diabetes (e.g., type 1 diabetes or type 2 diabetes) can be identified as likely to benefit from BCG therapy on the basis of the age of the subject at the time of onset of the diabetes. In particular, a diabetes subject that develops diabetes at a young age (e.g., an age of less than 40 years old at the time of onset of the diabetes, such as an age of 39 years, 38 years, 37 years, 36 years, 35 years, 34 years, 33 years, 32 years, 31 years, 30 years, 29 years, 28 years, 27 years, 26 years, 25 years, 24 years, 23 years, 22 years, 21 years, 20 years, 19 years, 18 years, 17 years, 16 years, 15 years, 14 years, 13 years, 12 years, 11 years, 10 years, 9 years, 8 years, 7 years, 6 years, 5 years, 4 years, or 3 years, or less, at the time of onset of the diabetes) has a greater likelihood of benefiting from BCG therapy as compared to a subject that develops diabetes at an older age (e.g., an age of greater than 40 year old). Surprisingly, a subject's current, chronological age and the duration of time during which a subject has been living with the diabetes are not predictive of a subject's responsiveness to BCG treatment. Thus, using the compositions and methods described herein, a diabetes subject can be assessed for their likelihood to rapidly respond to BCG therapy by determining the age of the subject at the time of onset of the diabetes, regardless of the current age of the subject or the total time during which the subject has been living with diabetes. In this way, a subject with a younger age at the time of onset of the diabetes (e.g., an age of less than 40 years at the time of onset of the diabetes, such as an age of 39 years, 38 years, 37 years, 36 years, 35 years, 34 years, 33 years, 32 years, 31 years, 30 years, 29 years, 28 years, 27 years, 26 years, 25 years, 24 years, 23 years, 22 years, 21 years, 20 years, 19 years, 18 years, 17 years, 16 years, 15 years, 14 years, 13 years, 12 years, 11 years, 10 years, 9 years, 8 years, 7 years, 6 years, 5 years, 4 years, or 3 years, or less, at the time of onset of the diabetes) can be identified as likely to benefit from BCG therapy and can be treated accordingly. Using the compositions and methods of the disclosure, a diabetes subject can be identified as likely to benefit from BCG therapy and treated accordingly, even if, for example, the subject is currently a child, young adult, or elderly adult, and/or even if the subject has been living with the diabetes for an extended period of time (e.g., greater than six months, 1, 2, 3, 4, 5, 6, 7, 8, 9, 10, 11, 12, 13, 14, or 15 years, or longer than 15 years), and/or even if the subject lacks any evidence of functional beta islet cells (based on c-peptide levels (e.g., as evidenced by a fasting or stimulated c-peptide concentration in blood of about 200 pM or less, such as a fasting or stimulated c-peptide concentration in blood of from about 1 pM to about 200 pM, from about 1 pM to about 100 pM, from about 1 pM to about 50 pM, from about 1 pM to about 25 pM, from about 1 pM to about 10 pM, or from about 1 pM to about 5 pM) and/or HbA1c levels (e.g., as evidenced by an HbA1c level of from about 6.5% to about 15%, or more, as defined using the Diabetes Control and Complications Trial (DCCT) unit of measurement, which is described in Day et al., The British Journal of Diabetes & Vascular Disease 9(3):2009, the disclosure of which is incorporated herein by reference), so long as the age of the subject at the time of onset of the diabetes was −40 years old or younger (e.g., preferably 30 years old or younger).

Additionally or alternatively, using the compositions and methods of the disclosure, a subject with diabetes (e.g., type 1 diabetes or type 2 diabetes) may be assessed for their likelihood to benefit from BCG therapy on the basis of the subject's pre-treatment level of glucose uptake (e.g., a glucose uptake of from about 6,000 mean fluorescence intensity (MFI) units to about 10,000 MFI, as assessed by way of a fluorescence intensity assay conducted by exposing a cell from the subject (e.g., a blood cell, such as a peripheral blood cell, such as a leukocyte, for example, a monocyte) to a fluorescently-labeled glucose analog, such as 2-[N-(7-nitrobenz-2-oxa-1,3-diazol-4-yl)amino]-2-deoxy-D-glucose (2-NBDG)). For example, a subject exhibiting reduced rates of glucose uptake (e.g., in peripheral blood cells, such as leukocytes, including, e.g., monocytes) relative to a reference rate of glucose uptake (e.g., a rate of glucose uptake that is less than a reference rate (e.g., from about 6,000 median fluorescence intensity (MFI) to about 10,000 MFI, as assessed by way of a fluorescently-labeled glucose uptake assay described herein) of glucose uptake by from about 5% to about 50%, or more), such as a rate of glucose uptake exhibited by a subject that does not have diabetes (e.g., such as a rate of glucose uptake exhibited in peripheral blood cells, such as leukocytes, including, e.g., monocytes, in a subject that does not have diabetes), is likely to benefit from BCG treatment. Accordingly, using the compositions and methods of the disclosure, a subject may be tested for their propensity to benefit from BCG therapy by determining a rate of glucose uptake exhibited by the subject, and this rate of glucose uptake may then be compared to a reference rate of glucose uptake (e.g., a rate of glucose uptake exhibited by a subject that does not have diabetes, such as a rate of glucose uptake exhibited in peripheral blood cells, such as leukocytes, including, e.g., monocytes, in a subject that does not have diabetes). A determination that the subject exhibits a reduced level of glucose uptake relative to such a reference level indicates that the subject is likely to respond to BCG treatment.

In an additional example, using the compositions and methods described herein, a subject with diabetes (e.g., type 1 diabetes or type 2 diabetes) may be assessed for their likelihood to benefit from BCG therapy on the basis of the subject's pre-treatment level of oxidative phosphorylation and/or aerobic glycolysis. A subject exhibiting overactive oxidative phosphorylation (e.g., in peripheral blood cells, such as leukocytes, including, e.g., monocytes) relative to a reference rate of oxidative phosphorylation, such as a rate of oxidative phosphorylation exhibited by a subject that does not have diabetes, is likely to rapidly benefit from BCG treatment. For example, a subject exhibiting a rate of oxidative phosphorylation that is increased by 5% to 200%, or more, relative to a rate of oxidative phosphorylation exhibited by a subject that does not have diabetes is likely to rapidly benefit from BCG treatment. In some embodiments, the reference rate of oxidative phosphorylation is from about 25 pmol of O₂ consumption per minute to about 150 pmol of O₂ consumption per minute, such as 25, 30, 35, 40, 45, 50, 55, 60, 65, 70, 75, 80, 85, 90, 95, 100, 105, 110, 115, 120, 125, 130, 135, 140, 145, or 150 pmol of O₂ consumption per minute. Similarly, a subject exhibiting reduced rates of aerobic glycolysis (e.g., in peripheral blood cells, such as leukocytes, including, e.g., monocytes) relative to a reference rate of aerobic glycolysis, such as a rate of aerobic glycolysis exhibited by a subject that does not have diabetes is likely to benefit from BCG treatment. In some embodiments, the subject exhibits a rate of aerobic glycolysis that is reduced by from about 5% to about 50%, or more, relative to the rate of aerobic glycolysis exhibited by a subject that does not have diabetes. In some embodiments, the reference rate of aerobic glycolysis is from about 5 mpH units per minute to about 50 mpH units per minute (e.g., a reference rate of about 5, 10, 15, 20, 25, 30, 35, 40, 45, or 50 mpH units per minute).

Accordingly, using the compositions and methods of the disclosure, a subject may be tested for their propensity to benefit from BCG therapy by determining a rate of oxidative phosphorylation and/or aerobic glycolysis exhibited by the subject, and comparing one or both of these quantities to a reference rate of oxidative phosphorylation and/or aerobic glycolysis (e.g., a rate of oxidative phosphorylation and/or aerobic glycolysis exhibited by a subject that does not have diabetes, such as a rate of oxidative phosphorylation and/or aerobic glycolysis exhibited in peripheral blood cells, such as leukocytes, including, e.g., monocytes, in a subject that does not have diabetes). A determination that the subject exhibits an elevated rate of oxidative phosphorylation (e.g., a rate of oxidative phosphorylation that is increased by 5% to 200%, or more relative to a reference rate of oxidative phosphorylation, such as a rate of oxidative phosphorylation exhibited by a subject that does not have diabetes (e.g., a reference rate of from about 25 pmol of O₂ consumption per minute to about 150 pmol of O₂ consumption per minute)) and/or a reduced rate of aerobic glycolysis relative to such a reference level (e.g., a level of aerobic glycolysis that is reduced by 5% to 50%, or more, relative to a reference rate of aerobic glycolysis, such as a rate of aerobic glycolysis exhibited by a subject that does not have diabetes (e.g., a reference rate of from about 5 mpH units per minute to about 50 mpH units per minute, such as about 5, 10, 15, 20, 25, 30, 35, 40, 45, or 50 mpH units per minute)) indicates that the subject is likely to respond to BCG treatment.

A subject with diabetes (e.g., type 1 diabetes or type 2 diabetes) may, additionally or alternatively, be assessed for their likelihood to benefit from BCG treatment using the compositions and methods described herein by examining the response of a sample of cells obtained from the subject to BCG exposure. In this context, a sample(s) of cells (e.g., peripheral blood cells, such as leukocytes, including, e.g., monocytes) obtained from the subject may be incubated in the presence of BCG (e.g., the sample(s) may be divided into two or more samples and a first sample can be tested in the presence of BCG and, optionally, a second sample can be tested in the absence of BCG as a control). A determination that the rate of glucose uptake by the cells increases, that the rate of oxidative phosphorylation in the cells decreases, and/or that the rate of aerobic glycolysis in the cells increases upon incubation with BCG (e.g., relative to a rate of glucose uptake, oxidative phosphorylation, and/or aerobic glycolysis exhibited by the cells prior to incubation with BCG) indicates that the subject is likely to benefit from treatment with BCG. For example, the rate of glucose uptake exhibited by the cells may increase by from about 5% to about 200%, or more (e.g., by about 5%, 25%, 50%, 100%, 150%, 200%, or more) upon incubation with BCG relative to the rate of glucose uptake exhibited by the cells prior to incubation with BCG. Additionally or alternatively, the rate of oxidative phosphorylation exhibited by the cells may decrease by from about 5% to about 50%, or more (e.g., by about 5%, 10%, 20%, 30%, 40%, 50%, or more) upon incubation with BCG relative to the rate of oxidative phosphorylation exhibited by the cells prior to incubation with BCG. Additionally or alternatively, the rate of aerobic glycolysis exhibited by the cells may increase by from about 5% to about 200%, or more (e.g., by about 5%, 25%, 50%, 100%, 150%, 200%, or more) upon incubation with BCG relative to the rate of aerobic glycolysis exhibited by the cells prior to incubation with BCG. Thus, using one or more of these assays, a diabetes subject may be identified as likely to rapidly benefit from BCG therapy (e.g., the subject is expected to exhibit one or more benefits from BCG therapy within about 1, 2, 3, 4, 5, or 6 months, or within about 1, 2, 3, 4, or 5 years after BCG administration) and, using the therapeutic compositions and methods described herein, the subject may be treated with accordingly with a composition comprising BCG, as described herein.

In a first aspect, the disclosure features a method of treating diabetes in a subject (e.g., a mammalian subject, such as a human subject) in need thereof by administering a therapeutically effective amount of BCG (e.g., about 1×10⁴ colony forming units (CFU) per 0.1 mg of BCG to about 1×10⁸ CFU per 0.1 mg of BCG, as described herein) to the subject. In some embodiments of this aspect, prior to administration of the BCG to the subject, the subject has been determined to:
a) be aged less than 40 years old at the time of onset of the diabetes (e.g., an age of 40 years, 39 years, 38 years, 37 years, 36 years, 35 years, 34 years, 33 years, 32 years, 31 years, 30 years, 29 years, 28 years, 27 years, 26 years, 25 years, 24 years, 23 years, 22 years, 21 years, 20 years, 19 years, 18 years, 17 years, 16 years, 15 years, 14 years, 13 years, 12 years, 11 years, 10 years, 9 years, 8 years, 7 years, 6 years, 5 years, 4 years, 3 years, 2 years, 1 year, or less, at the onset of the diabetes, such as an age of 20 years or less at the onset of the diabetes, e.g., an age of 20 years, 19 years, 18 years, 17 years, 16 years, 15 years, 14 years, 13 years, 12 years, 11 years, 10 years, 9 years, 8 years, 7 years, 6 years, 5 years, 4 years, 3 years, 2 years, 1 year, or less, at the onset of the diabetes), optionally where the subject is 40 years old or more at the time of being administered the BCG (e.g., 40, 41, 42, 43, 44, 45, 46, 47, 48, 49, 50, 51, 52, 53, 54, 55, 56, 57, 58, 59, 60, 61, 62, 63, 64, or 65 years old, or more, at the time of being administered the BCG);
b) exhibit a rate of glucose uptake that is less than a reference rate of glucose uptake, such as a reference rate of from about 6,000 MFI to about 10,000 MFI, as assessed by way of a fluorescently-labeled glucose uptake assay described herein (e.g., a rate of glucose uptake that is less than a reference rate of glucose uptake by 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, or more),
   i. optionally wherein the reference rate of glucose uptake is a rate of glucose uptake exhibited by a reference subject that does not have diabetes; (e.g., a mammalian subject, such as a human subject) that does not have diabetes, as assessed, for example, by evaluating the rate of glucose uptake in a sample of cells obtained from the reference subject, such as a sample of cells that includes peripheral blood cells (e.g., leukocytes, such as monocytes, neutrophils, basophils, eosinophils, and/or lymphocytes, (e.g., CD4+ lymphocytes)), or
   ii. optionally wherein the reference rate of glucose uptake is a reference rate standard (e.g., from about 6,000 MFI to about 10,000 MFI, as assessed by way of a fluorescently-labeled glucose uptake assay described herein) that is equivalent to a rate of glucose uptake exhibited by a healthy subject (e.g., a mammalian subject, such as a human subject) that does not have diabetes, such as a reference rate standard that is determined by analyzing data obtained from an experiment in which a rate of glucose uptake is determined in a sample of cells obtained from the healthy subject, such as a sample of cells that includes peripheral blood cells (e.g., leukocytes, such as monocytes, neutrophils, basophils, eosinophils, and/or lymphocytes, (e.g., CD4+ lymphocytes));
c) exhibit a rate of glucose uptake that is approximately equivalent to a rate of glucose uptake exhibited by a subject (e.g., a mammalian subject, such as a human subject) with diabetes and that is known to respond to treatment with BCG (e.g., a rate of glucose uptake that is within 5%, 4%, 3%, 2%, 1%, 0.9%, 0.8%, 0.7%, 0.6%, 0.5%, 0.4%, 0.3%, 0.2%, 0.1%, 0.05%, or less, of a rate of glucose uptake exhibited by a subject (e.g., a mammalian subject, such as a human subject) with diabetes and that is known to respond to treatment with BCG, such a rate of glucose update of from about 6,000 MFI to about 10,000 MFI, as assessed by a fluorescently-labeled glucose uptake assay, such as the one described herein),
   i. optionally wherein the rate of glucose uptake exhibited by the subject known to respond to treatment with BCG is assessed by evaluating the rate of glucose uptake in a sample of cells obtained from the subject known to respond to treatment with BCG, such as a sample of cells that includes peripheral blood cells (e.g., leukocytes, such as monocytes, neutrophils, basophils, eosinophils, and/or lymphocytes, (e.g., CD4+ lymphocytes));
d) exhibit a rate of oxidative phosphorylation that is greater than a reference rate of oxidative phosphorylation (e.g., a rate of oxidative phosphorylation that is greater than a reference rate of oxidative phosphorylation (e.g., a reference rate of from about 25 pmol of O₂ consumption per minute to about 150 pmol of O₂ consumption per minute) by 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 200%, 300%, 400%, 500%, or more),
   i. optionally wherein the reference rate of oxidative phosphorylation is a rate of oxidative phosphorylation exhibited by a reference subject that does not have diabetes; (e.g., a mammalian subject, such as a human subject) that does not have diabetes, as assessed, for example, by evaluating the rate of oxygen consumption in a sample of cells obtained from the reference subject, such as a sample of cells that includes peripheral blood cells (e.g., leukocytes, such as monocytes, neutrophils, basophils, eosinophils, and/or lymphocytes, (e.g., CD4+ lymphocytes)), or
   ii. optionally wherein the reference rate of oxidative phosphorylation is a reference rate standard that is equivalent to a rate of oxidative phosphorylation exhibited by a healthy subject (e.g., a mammalian subject, such as a human subject) that does not have diabetes, such as a reference rate standard that is determined by analyzing data obtained from an experiment in which a rate of oxygen consumption is determined in a sample of cells obtained from the healthy subject, such as a sample of cells that includes peripheral blood cells (e.g., leukocytes, such as monocytes, neutrophils, basophils, eosinophils, and/or lymphocytes, (e.g., CD4+ lymphocytes));
e) exhibit a rate of oxidative phosphorylation that is approximately equivalent to a rate of oxidative phosphorylation exhibited by a subject (e.g., a mammalian subject, such as a human subject) with diabetes and that is known to respond to treatment with BCG (e.g., a rate of oxidative phosphorylation that is within 5%, 4%, 3%, 2%, 1%, 0.9%, 0.8%, 0.7%, 0.6%, 0.5%, 0.4%, 0.3%, 0.2%, 0.1%, 0.05%, or less, of a rate of oxidative phosphorylation exhibited by a subject (e.g., a mammalian subject, such as a human subject) with diabetes and that is known to respond to treatment with BCG),
   i. optionally wherein the rate of oxidative phosphorylation exhibited by the subject known to respond to treatment with BCG is assessed by evaluating the rate of oxygen consumption in a sample of cells obtained from the subject known to respond to treatment with BCG, such as a sample of cells that includes peripheral blood cells (e.g., leukocytes, such as monocytes, neutrophils, basophils, eosinophils, and/or lymphocytes, (e.g., CD4+ lymphocytes));
f) exhibit a rate of aerobic glycolysis that is less than a reference rate of aerobic glycolysis, such as a reference rate of from about 5 mpH units per minute to about 50 mpH units per minute (e.g., a rate of aerobic glycolysis that is less than a reference rate of aerobic glycolysis by 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, or more),
   i. optionally wherein the reference rate of aerobic glycolysis is a rate of aerobic glycolysis exhibited by a reference subject that does not have diabetes; (e.g., a mammalian subject, such as a human subject) that does not have diabetes, as assessed, for example, by evaluating the rate of extracellular acidification in a sample of cells obtained from the reference subject, such as a sample of cells that includes peripheral blood cells (e.g., leukocytes, such as monocytes, neutrophils, basophils, eosinophils, and/or lymphocytes, (e.g., CD4+ lymphocytes)), or
   ii. optionally wherein the reference rate of aerobic glycolysis is a reference rate standard that is equivalent to a rate of aerobic glycolysis exhibited by a healthy subject (e.g., a mammalian subject, such as a human subject) that does not have diabetes, such as a reference rate standard that is determined by analyzing data obtained from an experiment in which a rate of extracellular acidification is determined in a sample of cells obtained from the healthy subject, such as a sample of cells that includes peripheral blood cells (e.g., leukocytes, such as monocytes, neutrophils, basophils, eosinophils, and/or lymphocytes, (e.g., CD4+ lymphocytes)); and/or
g) exhibit a rate of aerobic glycolysis that is approximately equivalent to a rate of aerobic glycolysis exhibited by a subject (e.g., a mammalian subject, such as a human subject) with diabetes and that is known to respond to treatment with BCG (e.g., a rate of aerobic glycolysis that is within 5%, 4%, 3%, 2%, 1%, 0.9%, 0.8%, 0.7%, 0.6%, 0.5%, 0.4%, 0.3%, 0.2%, 0.1%, 0.05%, or less, of a rate of aerobic glycolysis exhibited by a subject (e.g., a mammalian subject, such as a human subject) with diabetes and that is known to respond to treatment with BCG),
   i. optionally wherein the rate of aerobic glycolysis exhibited by the subject known to respond to treatment with BCG is assessed by evaluating the rate of extracellular acidification in a sample of cells obtained from the subject known to respond to treatment with BCG, such as a sample of cells that includes peripheral blood cells (e.g., leukocytes, such as monocytes, neutrophils, basophils, eosinophils, and/or lymphocytes, (e.g., CD4+ lymphocytes)).

In another aspect, the disclosure features a method of selecting a subject (e.g., a mammalian subject, such as a human subject) with diabetes for treatment with BCG by:
a) determining an age of onset of the diabetes in the subject, wherein a determination that the subject was less than 40 years old at the onset of the diabetes (e.g., a determination that the subject was 40 years old, 39 years old, 38 years old, 37 years old, 36 years old, 35 years old, 34 years old, 33 years old, 32 years old, 31 years old, 30 years old, 29 years old, 28 years old, 27 years old, 26 years old, 25 years old, 24 years old, 23 years old, 22 years old, 21 years old, 20 years old, 19 years old, 18 years old, 17 years old, 16 years old, 15 years old, 14 years old, 13 years old, 12 years old, 11 years old, 10 years old, 9 years old, 8 years old, 7 years old, 6 years old, 5 years old, 4 years old, 3 years old, 2 years old, 1 year old, or younger, at the onset of the diabetes, such as a determination that the subject was 20 years old, 19 years old, 18 years old, 17 years old, 16 years old, 15 years old, 14 years old, 13 years old, 12 years old, 11 years old, 10 years old, 9 years old, 8 years old, 7 years old, 6 years old, 5 years old, 4 years old, 3 years old, 2 years old, 1 year old, or younger) identifies the subject as likely to respond to the BCG;
b) determining a rate of glucose uptake exhibited by the subject, wherein a determination that the subject exhibits a rate of glucose uptake that is less than a reference rate of glucose uptake (e.g., a rate of glucose uptake that is less than a reference rate of glucose uptake by 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, or more) identifies the subject as likely to respond to the BCG,
   i. optionally wherein the reference rate of glucose uptake is a rate of glucose uptake exhibited by a reference subject that does not have diabetes; (e.g., a mammalian subject, such as a human subject) that does not have diabetes, as assessed, for example, by evaluating the rate of glucose uptake in a sample of cells obtained from the reference subject, such as a sample of cells that includes peripheral blood cells (e.g., leukocytes, such as monocytes, neutrophils, basophils, eosinophils, and/or lymphocytes, (e.g., CD4+ lymphocytes)), or
   ii. optionally wherein the reference rate of glucose uptake is a reference rate standard that is equivalent to a rate of glucose uptake exhibited by a healthy subject (e.g., a mammalian subject, such as a human subject) that does not have diabetes, such as a reference rate standard that is determined by analyzing data obtained from an experiment in which a rate of glucose uptake is determined in a sample of cells obtained from the healthy subject, such as a sample of cells that includes peripheral blood cells (e.g., leukocytes, such as monocytes, neutrophils, basophils, eosinophils, and/or lymphocytes, (e.g., CD4+ lymphocytes));
c) determining a rate of glucose uptake exhibited by the subject, wherein a determination that the subject exhibits a rate of glucose uptake that is approximately equivalent to a rate of glucose uptake exhibited by a subject (e.g., a mammalian subject, such as a human subject) with diabetes and that is known to respond to treatment with BCG (e.g., a rate of glucose uptake that is within 5%, 4%, 3%, 2%, 1%, 0.9%, 0.8%, 0.7%, 0.6%, 0.5%, 0.4%, 0.3%, 0.2%, 0.1%, 0.05%, or less, of a rate of glucose uptake exhibited by a subject (e.g., a mammalian subject, such as a human subject) with diabetes and that is known to respond to treatment with BCG) identifies the subject as likely to respond to the BCG,
   i. optionally wherein the rate of glucose uptake exhibited by the subject known to respond to treatment with BCG is assessed by evaluating the rate of glucose uptake in a sample of cells obtained from the subject known to respond to treatment with BCG, such as a sample of cells that includes peripheral blood cells (e.g., leukocytes, such as monocytes, neutrophils, basophils, eosinophils, and/or lymphocytes, (e.g., CD4+ lymphocytes));
d) determining a rate of oxidative phosphorylation exhibited by the subject, wherein a determination that the subject exhibits a rate of oxidative phosphorylation that is greater than a reference rate of oxidative phosphorylation (e.g., a rate of oxidative phosphorylation that is greater than a reference rate of oxidative phosphorylation by 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 200%, 300%, 400%, 500%, or more) identifies the subject as likely to respond to the BCG,
   i. optionally wherein the reference rate of oxidative phosphorylation is a rate of oxidative phosphorylation exhibited by a reference subject that does not have diabetes; (e.g., a mammalian subject, such as a human subject) that does not have diabetes, as assessed, for example, by evaluating the rate of oxygen consumption in a sample of cells obtained from the reference subject, such as a sample of cells that includes peripheral blood cells (e.g., leukocytes, such as monocytes, neutrophils, basophils, eosinophils, and/or lymphocytes, (e.g., CD4+ lymphocytes)), or
   ii. optionally wherein the reference rate of oxidative phosphorylation is a reference rate standard that is equivalent to a rate of oxidative phosphorylation exhibited by a healthy subject (e.g., a mammalian subject, such as a human subject) that does not have diabetes, such as a reference rate standard that is determined by analyzing data obtained from an experiment in which a rate of oxygen consumption is determined in a sample of cells obtained from the healthy subject, such as a sample of cells that includes peripheral blood cells (e.g., leukocytes, such as monocytes, neutrophils, basophils, eosinophils, and/or lymphocytes, (e.g., CD4+ lymphocytes));
e) determining a rate of oxidative phosphorylation exhibited by the subject, wherein a determination that the subject exhibits a rate of oxidative phosphorylation that is approximately equivalent to a rate of oxidative phosphorylation exhibited by a subject (e.g., a mammalian subject, such as a human subject) with diabetes and that is known to respond to treatment with BCG (e.g., a rate of oxidative phosphorylation that is within 5%, 4%, 3%, 2%, 1%, 0.9%, 0.8%, 0.7%, 0.6%, 0.5%, 0.4%, 0.3%, 0.2%, 0.1%, 0.05%, or less, of a rate of oxidative phosphorylation exhibited by a subject (e.g., a mammalian subject, such as a human subject) with diabetes and that is known to respond to treatment with BCG) identifies the subject as likely to respond to the BCG,
   i. optionally wherein the rate of oxidative phosphorylation exhibited by the subject known to respond to treatment with BCG is assessed by evaluating the rate of oxygen consumption in a sample of cells obtained from the subject known to respond to treatment with BCG, such as a sample of cells that includes peripheral blood cells (e.g., leukocytes, such as monocytes, neutrophils, basophils, eosinophils, and/or lymphocytes, (e.g., CD4+ lymphocytes));
f) determining a rate of aerobic glycolysis exhibited by the subject, wherein a determination that the subject exhibits a rate of aerobic glycolysis that is less than a reference rate of aerobic glycolysis (e.g., a rate of aerobic glycolysis that is less than a reference rate of aerobic glycolysis by 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, or more) identifies the subject as likely to respond to the BCG,
   i. optionally wherein the reference rate of aerobic glycolysis is a rate of aerobic glycolysis exhibited by a reference subject that does not have diabetes; (e.g., a mammalian subject, such as a human subject) that does not have diabetes, as assessed, for example, by evaluating the rate of extracellular acidification in a sample of cells obtained from the reference subject, such as a sample of cells that includes peripheral blood cells (e.g., leukocytes, such as monocytes, neutrophils, basophils, eosinophils, and/or lymphocytes, (e.g., CD4+ lymphocytes)), or
   ii. optionally wherein the reference rate of aerobic glycolysis is a reference rate standard that is equivalent to a rate of aerobic glycolysis exhibited by a healthy subject (e.g., a mammalian subject, such as a human subject) that does not have diabetes, such as a reference rate standard that is determined by analyzing data obtained from an experiment in which a rate of extracellular acidification is determined in a sample of cells obtained from the healthy subject, such as a sample of cells that includes peripheral blood cells (e.g., leukocytes, such as monocytes, neutrophils, basophils, eosinophils, and/or lymphocytes, (e.g., CD4+ lymphocytes; and/or
g) determining a rate of aerobic glycolysis exhibited by the subject, wherein a determination that the subject exhibits a rate of aerobic glycolysis that is approximately equivalent to a rate of aerobic glycolysis exhibited by a subject (e.g., a mammalian subject, such as a human subject) with diabetes and that is known to respond to treatment with BCG (e.g., a rate of aerobic glycolysis that is within 5%, 4%, 3%, 2%, 1%, 0.9%, 0.8%, 0.7%, 0.6%, 0.5%, 0.4%, 0.3%, 0.2%, 0.1%, 0.05%, or less, of a rate of aerobic glycolysis exhibited by a subject (e.g., a mammalian subject, such as a human subject) with diabetes and that is known to respond to treatment with BCG) identifies the subject as likely to respond to the BCG,
   i. optionally wherein the rate of aerobic glycolysis exhibited by the subject known to respond to treatment with BCG is assessed by evaluating the rate of extracellular acidification in a sample of cells obtained from the subject known to respond to treatment with BCG, such as a sample of cells that includes peripheral blood cells (e.g., leukocytes, such as monocytes, neutrophils, basophils, eosinophils, and/or lymphocytes, (e.g., CD4+ lymphocytes)).
      In some embodiments of this aspect, the method further includes administering a therapeutically effective amount of BCG to the subject identified as likely to respond to the BCG.

In another aspect, the disclosure features a method of treating diabetes in a subject (e.g., a mammalian subject, such as a human subject) in need thereof by administering a therapeutically effective amount of BCG to the subject, wherein the subject has been selected for treatment with BCG by a method that includes:
a) providing a sample of cells (e.g., peripheral blood cells, such as leukocytes (e.g., monocytes)) obtained from the subject prior to administration of the BCG to the subject;
b) culturing the sample of cells in the presence of BCG; and
c) evaluating a rate of glucose uptake in the sample of cells.
   In some embodiments of this aspect, the rate of glucose uptake in the sample of cells is determined to be greater than a reference rate of glucose uptake (e.g., by 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 200%, 300%, 400%, 500%, or more). The reference rate of glucose uptake may be, for example, a rate of glucose uptake that has been determined in a sample of cells obtained from the subject previously and that has not been exposed to BCG.

In some embodiments of this aspect, the rate of glucose uptake in the sample of cells is determined to be approximately equivalent (e.g., within 5%, 4%, 3%, 2%, 1%, 0.9%, 0.8%, 0.7%, 0.6%, 0.5%, 0.4%, 0.3%, 0.2%, 0.1%, or less) to a rate of glucose uptake that has been determined in a sample of cells obtained from a reference subject (e.g., a mammalian subject, such as a human subject) that does not have diabetes.

In some embodiments of this aspect, the subject has been selected for treatment with BCG by a method that includes:
a) providing first and second samples of cells (e.g., peripheral blood cells, such as leukocytes (e.g., monocytes)) obtained from the subject prior to administration of the BCG to the subject;
b) culturing the first sample of cells in the absence of BCG and the second sample of cells in the presence of BCG; and
c) evaluating a rate of glucose uptake in the first and second samples of cells.
   In some embodiments, the rate of glucose uptake in the second sample of cells is determined to be greater than the rate of glucose uptake in the first sample of cells (e.g., by 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 200%, 300%, 400%, 500%, or more).

In another aspect, the disclosure features a method of selecting a subject (e.g., a mammalian subject, such as a human subject) with diabetes for treatment with BCG by:
a) providing a sample of cells (e.g., peripheral blood cells, such as leukocytes (e.g., monocytes)) obtained from the subject prior to administration of BCG to the subject;
b) culturing the sample of cells in the presence of BCG; and
c) evaluating a rate of glucose uptake in sample of cells.
   In some embodiments of this aspect, a determination that the rate of glucose uptake in the sample of cells is greater than a reference rate of glucose uptake (e.g., by 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 200%, 300%, 400%, 500%, or more) identifies the subject as likely to respond to the BCG. The reference rate of glucose uptake may be, for example, a rate of glucose uptake that has been determined in a sample of cells obtained from the subject previously and that has not been exposed to BCG.

In some embodiments of this aspect, a determination that the rate of glucose uptake in the sample of cells is approximately equivalent (e.g., within 5%, 4%, 3%, 2%, 1%, 0.9%, 0.8%, 0.7%, 0.6%, 0.5%, 0.4%, 0.3%, 0.2%, 0.1%, or less) to a rate of glucose uptake that has been determined in a sample of cells obtained from a reference subject (e.g., a mammalian subject, such as a human subject) that does not have diabetes identifies the subject as likely to respond to the BCG.

In some embodiments of this aspect, the method includes:
a) providing first and second samples of cells obtained from the subject prior to administration of BCG to the subject;
b) culturing the first sample of cells in the absence of BCG and the second sample of cells in the presence of BCG; and
c) evaluating a rate of glucose uptake in the first and second samples of cells.
   In some embodiments, a determination that the rate of glucose uptake in the second sample of cells is greater than the rate of glucose uptake in the first sample of cells (e.g., by 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 200%, 300%, 400%, 500%, or more) identifies the subject as likely to respond to the BCG. In some embodiments, the method further includes administering a therapeutically effective amount of BCG to the subject identified as likely to respond to the BCG.

In a further aspect, the disclosure features a method of treating diabetes in a subject (e.g., a mammalian subject, such as a human subject) in need thereof by administering a therapeutically effective amount of BCG to the subject, wherein the subject has been selected for treatment with BCG by a method that includes:
a) providing a sample of cells (e.g., peripheral blood cells, such as leukocytes (e.g., monocytes)) obtained from the subject prior to administration of the BCG to the subject;
b) culturing the sample of cells in the presence of BCG; and
c) evaluating a rate of oxidative phosphorylation in sample of cells.
   In some embodiments of this aspect, the rate of oxidative phosphorylation in the sample of cells is determined to be less than a reference rate of oxidative phosphorylation (e.g., by 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, or more). The reference rate of oxidative phosphorylation may be, for example, a rate of oxidative phosphorylation that has been determined in a sample of cells obtained from the subject previously and that has not been exposed to BCG.

In some embodiments of this aspect, the rate of oxidative phosphorylation in the sample of cells is determined to be approximately equivalent (e.g., within 5%, 4%, 3%, 2%, 1%, 0.9%, 0.8%, 0.7%, 0.6%, 0.5%, 0.4%, 0.3%, 0.2%, 0.1%, or less) to a rate of oxidative phosphorylation that has been determined in a sample of cells obtained from a reference subject (e.g., a mammalian subject, such as a human subject) that does not have diabetes.

In some embodiments of this aspect, the subject has been selected for treatment with BCG by a method that includes:
a) providing first and second samples of cells obtained from the subject prior to administration of the BCG to the subject;
b) culturing the first sample of cells in the absence of BCG and the second sample of cells in the presence of BCG; and
c) evaluating a rate of oxidative phosphorylation in the first and second samples of cells.
   In some embodiments, the rate of oxidative phosphorylation in the second sample of cells is determined to be less than the rate of oxidative phosphorylation in the first sample of cells (e.g., by 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, or more).

In another aspect, the disclosure features a method of selecting a subject (e.g., a mammalian subject, such as a human subject) with diabetes for treatment with BCG by:
a) providing a sample of cells obtained from the subject prior to administration of the BCG to the subject;
b) culturing the sample of cells in the presence of BCG; and
c) evaluating a rate of oxidative phosphorylation in the sample of cells.

In some embodiments of this aspect, a determination that the rate of oxidative phosphorylation in the sample of cells is less than a reference rate of oxidative phosphorylation (e.g., by 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, or more) identifies the subject as likely to respond to the BCG. The reference rate of oxidative phosphorylation may be, for example, a rate of oxidative phosphorylation that has been determined in a sample of cells obtained from the subject previously and that has not been exposed to BCG.

In some embodiments of this aspect, a determination that the rate of oxidative phosphorylation in the sample of cells is approximately equivalent (e.g., within 5%, 4%, 3%, 2%, 1%, 0.9%, 0.8%, 0.7%, 0.6%, 0.5%, 0.4%, 0.3%, 0.2%, 0.1%, or less) to a rate of oxidative phosphorylation that has been determined in a sample of cells obtained from a reference subject (e.g., a mammalian subject, such as a human subject) that does not have diabetes identifies the subject as likely to respond to the BCG.

In some embodiments of this aspect, the method includes:
a) providing first and second samples of cells obtained from the subject prior to administration of the BCG to the subject;
b) culturing the first sample of cells in the absence of BCG and the second sample of cells in the presence of BCG; and
c) evaluating a rate of oxidative phosphorylation in the first and second samples of cells.
   In some embodiments, a determination that the rate of oxidative phosphorylation in the second sample of cells is less than the rate of oxidative phosphorylation in the first sample of cells (e.g., by 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, or more) identifies the subject as likely to respond to the BCG. In some embodiments, the method further includes administering a therapeutically effective amount of BCG to the subject identified as likely to respond to the BCG.

In another aspect, the disclosure features a method of treating diabetes in a subject (e.g., a mammalian subject, such as a human subject) in need thereof by administering a therapeutically effective amount of BCG to the subject, wherein the subject has been selected for treatment with BCG by a method that includes:
a) providing a sample of cells obtained from the subject prior to administration of the BCG to the subject;
b) culturing the sample of cells in the presence of BCG; and
c) evaluating a rate of aerobic glycolysis in the sample of cells.
   In some embodiments of this aspect, the rate of aerobic glycolysis in the sample of cells is determined to be greater than a reference rate of aerobic glycolysis (e.g., by 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 200%, 300%, 400%, 500%, or more). The reference rate of aerobic glycolysis may be, for example, a rate of aerobic glycolysis that has been determined in a sample of cells obtained from the subject previously and that has not been exposed to BCG.

In some embodiments of this aspect, the rate of aerobic glycolysis in the sample of cells is determined to be approximately equivalent (e.g., within 5%, 4%, 3%, 2%, 1%, 0.9%, 0.8%, 0.7%, 0.6%, 0.5%, 0.4%, 0.3%, 0.2%, 0.1%, or less) to a rate of aerobic glycolysis that has been determined in a sample of cells obtained from a reference subject (e.g., a mammalian subject, such as a human subject) that does not have diabetes.

In some embodiments of this aspect, the subject has been selected for treatment with BCG by a method that includes:
a) providing first and second samples of cells (e.g., peripheral blood cells, such as leukocytes (e.g., monocytes)) obtained from the subject prior to administration of the BCG to the subject;
b) culturing the first sample of cells in the absence of BCG and the second sample of cells in the presence of BCG; and
c) evaluating a rate of aerobic glycolysis in the first and second samples of cells.
   In some embodiments, the rate of aerobic glycolysis in the second sample of cells is determined to be greater than the rate of aerobic glycolysis in the first sample of cells (e.g., by 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 200%, 300%, 400%, 500%, or more).

In another aspect, the disclosure features a method of selecting a subject (e.g., a mammalian subject, such as a human subject) with diabetes for treatment with BCG by:
a) providing a sample of cells obtained from the subject prior to administration of the BCG to the subject;
b) culturing the sample of cells in the presence of BCG; and
c) evaluating a rate of aerobic glycolysis in the sample of cells.

In some embodiments of this aspect, a determination that the rate of aerobic glycolysis in the sample of cells is greater than a reference rate of aerobic glycolysis (e.g., by 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 200%, 300%, 400%, 500%, or more) identifies the subject as likely to respond to the BCG. The reference rate of aerobic glycolysis may be, for example, a rate of aerobic glycolysis that has been determined in a sample of cells obtained from the subject previously and that has not been exposed to BCG.

In some embodiments of this aspect, a determination that the rate of aerobic glycolysis in the sample of cells is approximately equivalent (e.g., within 5%, 4%, 3%, 2%, 1%, 0.9%, 0.8%, 0.7%, 0.6%, 0.5%, 0.4%, 0.3%, 0.2%, 0.1%, or less) to a rate of aerobic glycolysis that has been determined in a sample of cells obtained from a reference subject (e.g., a mammalian subject, such as a human subject) that does not have diabetes identifies the subject as likely to respond to the BCG.

In some embodiments of this aspect, the method includes:
a) providing first and second samples of cells (e.g., peripheral blood cells, such as leukocytes (e.g., monocytes)) obtained from the subject prior to administration of the BCG to the subject;
b) culturing the first sample of cells in the absence of BCG and the second sample of cells in the presence of BCG; and subsequently
c) evaluating a rate of aerobic glycolysis in the first and second samples of cells.
   In some embodiments, a determination that the rate of aerobic glycolysis in the second sample of cells is greater than the rate of aerobic glycolysis in the first sample of cells (e.g., by 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 200%, 300%, 400%, 500%, or more) identifies the subject as likely to respond to the BCG. In some embodiments, the method further includes administering a therapeutically effective amount of BCG to the subject identified as likely to respond to the BCG.

In some embodiments of any of the foregoing aspects of the disclosure, the subject is or was determined to be 37 years old or younger at the onset of the diabetes (e.g., the subject is or was determined to be 37 years old or younger, 36 years old or younger, 35 years old or younger, 34 years old or younger, 33 years old or younger, 32 years old or younger, 31 years old or younger, 30 years old or younger, 29 years old or younger, 28 years old or younger, 27 years old or younger, 26 years old or younger, 25 years old or younger, 24 years old or younger, 23 years old or younger, 22 years old or younger, 21 years old or younger, 20 years old or younger, 19 years old or younger, 18 years old or younger, 17 years old or younger, 16 years old or younger, 15 years old or younger, 14 years old or younger, 13 years old or younger, 12 years old or younger, 11 years old or younger, 10 years old or younger, 9 years old or younger, 8 years old or younger, 7 years old or younger, 6 years old or younger, 5 years old or younger, 4 years old or younger, 3 years old or younger, 2 years old or younger, or 1 year old or younger at the onset of the diabetes).

For example, in some embodiments, the subject is or was determined to be 36 years old or younger at the onset of the diabetes (e.g., 36 years old or younger, 35 years old or younger, 34 years old or younger, 33 years old or younger, 32 years old or younger, 31 years old or younger, 30 years old or younger, 29 years old or younger, 28 years old or younger, 27 years old or younger, 26 years old or younger, 25 years old or younger, 24 years old or younger, 23 years old or younger, 22 years old or younger, 21 years old or younger, 20 years old or younger, 19 years old or younger, 18 years old or younger, 17 years old or younger, 16 years old or younger, 15 years old or younger, 14 years old or younger, 13 years old or younger, 12 years old or younger, 11 years old or younger, 10 years old or younger, 9 years old or younger, 8 years old or younger, 7 years old or younger, 6 years old or younger, 5 years old or younger, 4 years old or younger, 3 years old or younger, 2 years old or younger, or 1 year old or younger at the onset of the diabetes).

In some embodiments, the subject is or was determined to be 35 years old or younger at the onset of the diabetes (e.g., 35 years old or younger, 34 years old or younger, 33 years old or younger, 32 years old or younger, 31 years old or younger, 30 years old or younger, 29 years old or younger, 28 years old or younger, 27 years old or younger, 26 years old or younger, 25 years old or younger, 24 years old or younger, 23 years old or younger, 22 years old or younger, 21 years old or younger, 20 years old or younger, 19 years old or younger, 18 years old or younger, 17 years old or younger, 16 years old or younger, 15 years old or younger, 14 years old or younger, 13 years old or younger, 12 years old or younger, 11 years old or younger, 10 years old or younger, 9 years old or younger, 8 years old or younger, 7 years old or younger, 6 years old or younger, 5 years old or younger, 4 years old or younger, 3 years old or younger, 2 years old or younger, or 1 year old or younger at the onset of the diabetes).

In some embodiments, the subject is or was determined to be 34 years old or younger at the onset of the diabetes (e.g., 34 years old or younger, 33 years old or younger, 32 years old or younger, 31 years old or younger, 30 years old or younger, 29 years old or younger, 28 years old or younger, 27 years old or younger, 26 years old or younger, 25 years old or younger, 24 years old or younger, 23 years old or younger, 22 years old or younger, 21 years old or younger, 20 years old or younger, 19 years old or younger, 18 years old or younger, 17 years old or younger, 16 years old or younger, 15 years old or younger, 14 years old or younger, 13 years old or younger, 12 years old or younger, 11 years old or younger, 10 years old or younger, 9 years old or younger, 8 years old or younger, 7 years old or younger, 6 years old or younger, 5 years old or younger, 4 years old or younger, 3 years old or younger, 2 years old or younger, or 1 year old or younger at the onset of the diabetes).

In some embodiments, the subject is or was determined to be 33 years old or younger at the onset of the diabetes (e.g., 33 years old or younger, 32 years old or younger, 31 years old or younger, 30 years old or younger, 29 years old or younger, 28 years old or younger, 27 years old or younger, 26 years old or younger, 25 years old or younger, 24 years old or younger, 23 years old or younger, 22 years old or younger, 21 years old or younger, 20 years old or younger, 19 years old or younger, 18 years old or younger, 17 years old or younger, 16 years old or younger, 15 years old or younger, 14 years old or younger, 13 years old or younger, 12 years old or younger, 11 years old or younger, 10 years old or younger, 9 years old or younger, 8 years old or younger, 7 years old or younger, 6 years old or younger, 5 years old or younger, 4 years old or younger, 3 years old or younger, 2 years old or younger, or 1 year old or younger at the onset of the diabetes).

In some embodiments, the subject is or was determined to be 32 years old or younger at the onset of the diabetes (e.g., 32 years old or younger, 31 years old or younger, 30 years old or younger, 29 years old or younger, 28 years old or younger, 27 years old or younger, 26 years old or younger, 25 years old or younger, 24 years old or younger, 23 years old or younger, 22 years old or younger, 21 years old or younger, 20 years old or younger, 19 years old or younger, 18 years old or younger, 17 years old or younger, 16 years old or younger, 15 years old or younger, 14 years old or younger, 13 years old or younger, 12 years old or younger, 11 years old or younger, 10 years old or younger, 9 years old or younger, 8 years old or younger, 7 years old or younger, 6 years old or younger, 5 years old or younger, 4 years old or younger, 3 years old or younger, 2 years old or younger, or 1 year old or younger at the onset of the diabetes).

In some embodiments, the subject is or was determined to be 31 years old or younger at the onset of the diabetes (e.g., 31 years old or younger, 30 years old or younger, 29 years old or younger, 28 years old or younger, 27 years old or younger, 26 years old or younger, 25 years old or younger, 24 years old or younger, 23 years old or younger, 22 years old or younger, 21 years old or younger, 20 years old or younger, 19 years old or younger, 18 years old or younger, 17 years old or younger, 16 years old or younger, 15 years old or younger, 14 years old or younger, 13 years old or younger, 12 years old or younger, 11 years old or younger, 10 years old or younger, 9 years old or younger, 8 years old or younger, 7 years old or younger, 6 years old or younger, 5 years old or younger, 4 years old or younger, 3 years old or younger, 2 years old or younger, or 1 year old or younger at the onset of the diabetes).

In some embodiments, the subject is or was determined to be 30 years old or younger at the onset of the diabetes (e.g., 30 years old or younger, 29 years old or younger, 28 years old or younger, 27 years old or younger, 26 years old or younger, 25 years old or younger, 24 years old or younger, 23 years old or younger, 22 years old or younger, 21 years old or younger, 20 years old or younger, 19 years old or younger, 18 years old or younger, 17 years old or younger, 16 years old or younger, 15 years old or younger, 14 years old or younger, 13 years old or younger, 12 years old or younger, 11 years old or younger, 10 years old or younger, 9 years old or younger, 8 years old or younger, 7 years old or younger, 6 years old or younger, 5 years old or younger, 4 years old or younger, 3 years old or younger, 2 years old or younger, or 1 year old or younger at the onset of the diabetes).

In some embodiments, the subject is or was determined to be 29 years old or younger at the onset of the diabetes (e.g., 29 years old or younger, 28 years old or younger, 27 years old or younger, 26 years old or younger, 25 years old or younger, 24 years old or younger, 23 years old or younger, 22 years old or younger, 21 years old or younger, 20 years old or younger, 19 years old or younger, 18 years old or younger, 17 years old or younger, 16 years old or younger, 15 years old or younger, 14 years old or younger, 13 years old or younger, 12 years old or younger, 11 years old or younger, 10 years old or younger, 9 years old or younger, 8 years old or younger, 7 years old or younger, 6 years old or younger, 5 years old or younger, 4 years old or younger, 3 years old or younger, 2 years old or younger, or 1 year old or younger at the onset of the diabetes).

In some embodiments, the subject is or was determined to be 28 years old or younger at the onset of the diabetes (e.g., 28 years old or younger, 27 years old or younger, 26 years old or younger, 25 years old or younger, 24 years old or younger, 23 years old or younger, 22 years old or younger, 21 years old or younger, 20 years old or younger, 19 years old or younger, 18 years old or younger, 17 years old or younger, 16 years old or younger, 15 years old or younger, 14 years old or younger, 13 years old or younger, 12 years old or younger, 11 years old or younger, 10 years old or younger, 9 years old or younger, 8 years old or younger, 7 years old or younger, 6 years old or younger, 5 years old or younger, 4 years old or younger, 3 years old or younger, 2 years old or younger, or 1 year old or younger at the onset of the diabetes).

In some embodiments, the subject is or was determined to be 27 years old or younger at the onset of the diabetes (e.g., 28 years old or younger, 27 years old or younger, 26 years old or younger, 25 years old or younger, 24 years old or younger, 23 years old or younger, 22 years old or younger, 21 years old or younger, 20 years old or younger, 19 years old or younger, 18 years old or younger, 17 years old or younger, 16 years old or younger, 15 years old or younger, 14 years old or younger, 13 years old or younger, 12 years old or younger, 11 years old or younger, 10 years old or younger, 9 years old or younger, 8 years old or younger, 7 years old or younger, 6 years old or younger, 5 years old or younger, 4 years old or younger, 3 years old or younger, 2 years old or younger, or 1 year old or younger at the onset of the diabetes).

In some embodiments, the subject is or was determined to be 26 years old or younger at the onset of the diabetes (e.g., 26 years old or younger, 25 years old or younger, 24 years old or younger, 23 years old or younger, 22 years old or younger, 21 years old or younger, 20 years old or younger, 19 years old or younger, 18 years old or younger, 17 years old or younger, 16 years old or younger, 15 years old or younger, 14 years old or younger, 13 years old or younger, 12 years old or younger, 11 years old or younger, 10 years old or younger, 9 years old or younger, 8 years old or younger, 7 years old or younger, 6 years old or younger, 5 years old or younger, 4 years old or younger, 3 years old or younger, 2 years old or younger, or 1 year old or younger at the onset of the diabetes).

In some embodiments, the subject is or was determined to be 25 years old or younger at the onset of the diabetes (e.g., 25 years old or younger, 24 years old or younger, 23 years old or younger, 22 years old or younger, 21 years old or younger, 20 years old or younger, 19 years old or younger, 18 years old or younger, 17 years old or younger, 16 years old or younger, 15 years old or younger, 14 years old or younger, 13 years old or younger, 12 years old or younger, 11 years old or younger, 10 years old or younger, 9 years old or younger, 8 years old or younger, 7 years old or younger, 6 years old or younger, 5 years old or younger, 4 years old or younger, 3 years old or younger, 2 years old or younger, or 1 year old or younger at the onset of the diabetes).

In some embodiments, the subject is or was determined to be 24 years old or younger at the onset of the diabetes (e.g., 24 years old or younger, 23 years old or younger, 22 years old or younger, 21 years old or younger, 20 years old or younger, 19 years old or younger, 18 years old or younger, 17 years old or younger, 16 years old or younger, 15 years old or younger, 14 years old or younger, 13 years old or younger, 12 years old or younger, 11 years old or younger, 10 years old or younger, 9 years old or younger, 8 years old or younger, 7 years old or younger, 6 years old or younger, 5 years old or younger, 4 years old or younger, 3 years old or younger, 2 years old or younger, or 1 year old or younger at the onset of the diabetes).

In some embodiments, the subject is or was determined to be 23 years old or younger at the onset of the diabetes (e.g., 23 years old or younger, 22 years old or younger, 21 years old or younger, 20 years old or younger, 19 years old or younger, 18 years old or younger, 17 years old or younger, 16 years old or younger, 15 years old or younger, 14 years old or younger, 13 years old or younger, 12 years old or younger, 11 years old or younger, 10 years old or younger, 9 years old or younger, 8 years old or younger, 7 years old or younger, 6 years old or younger, 5 years old or younger, 4 years old or younger, 3 years old or younger, 2 years old or younger, or 1 year old or younger at the onset of the diabetes).

In some embodiments, the subject is or was determined to be 22 years old or younger at the onset of the diabetes (e.g., 22 years old or younger, 21 years old or younger, 20 years old or younger, 19 years old or younger, 18 years old or younger, 17 years old or younger, 16 years old or younger, 15 years old or younger, 14 years old or younger, 13 years old or younger, 12 years old or younger, 11 years old or younger, 10 years old or younger, 9 years old or younger, 8 years old or younger, 7 years old or younger, 6 years old or younger, 5 years old or younger, 4 years old or younger, 3 years old or younger, 2 years old or younger, or 1 year old or younger at the onset of the diabetes).

In some embodiments, the subject is or was determined to be 21 years old or younger at the onset of the diabetes (e.g., 21 years old or younger, 20 years old or younger, 19 years old or younger, 18 years old or younger, 17 years old or younger, 16 years old or younger, 15 years old or younger, 14 years old or younger, 13 years old or younger, 12 years old or younger, 11 years old or younger, 10 years old or younger, 9 years old or younger, 8 years old or younger, 7 years old or younger, 6 years old or younger, 5 years old or younger, 4 years old or younger, 3 years old or younger, 2 years old or younger, or 1 year old or younger at the onset of the diabetes).

In some embodiments, the subject is or was determined to be 20 years old or younger at the onset of the diabetes (e.g., 20 years old or younger, 19 years old or younger, 18 years old or younger, 17 years old or younger, 16 years old or younger, 15 years old or younger, 14 years old or younger, 13 years old or younger, 12 years old or younger, 11 years old or younger, 10 years old or younger, 9 years old or younger, 8 years old or younger, 7 years old or younger, 6 years old or younger, 5 years old or younger, 4 years old or younger, 3 years old or younger, 2 years old or younger, or 1 year old or younger at the onset of the diabetes).

In some embodiments, the subject is or was determined to be 19 years old or younger at the onset of the diabetes (e.g., 19 years old or younger, 18 years old or younger, 17 years old or younger, 16 years old or younger, 15 years old or younger, 14 years old or younger, 13 years old or younger, 12 years old or younger, 11 years old or younger, 10 years old or younger, 9 years old or younger, 8 years old or younger, 7 years old or younger, 6 years old or younger, 5 years old or younger, 4 years old or younger, 3 years old or younger, 2 years old or younger, or 1 year old or younger at the onset of the diabetes).

In some embodiments, the subject is or was determined to be 18 years old or younger at the onset of the diabetes (e.g., 18 years old or younger, 17 years old or younger, 16 years old or younger, 15 years old or younger, 14 years old or younger, 13 years old or younger, 12 years old or younger, 11 years old or younger, 10 years old or younger, 9 years old or younger, 8 years old or younger, 7 years old or younger, 6 years old or younger, 5 years old or younger, 4 years old or younger, 3 years old or younger, 2 years old or younger, or 1 year old or younger at the onset of the diabetes).

In some embodiments, the subject is or was determined to be 17 years old or younger at the onset of the diabetes (e.g., 17 years old or younger, 16 years old or younger, 15 years old or younger, 14 years old or younger, 13 years old or younger, 12 years old or younger, 11 years old or younger, 10 years old or younger, 9 years old or younger, 8 years old or younger, 7 years old or younger, 6 years old or younger, 5 years old or younger, 4 years old or younger, 3 years old or younger, 2 years old or younger, or 1 year old or younger at the onset of the diabetes).

In some embodiments, the subject is or was determined to be 16 years old or younger at the onset of the diabetes (e.g., 16 years old or younger, 15 years old or younger, 14 years old or younger, 13 years old or younger, 12 years old or younger, 11 years old or younger, 10 years old or younger, 9 years old or younger, 8 years old or younger, 7 years old or younger, 6 years old or younger, 5 years old or younger, 4 years old or younger, 3 years old or younger, 2 years old or younger, or 1 year old or younger at the onset of the diabetes).

In some embodiments, the subject is or was determined to be 15 years old or younger at the onset of the diabetes (e.g., 15 years old or younger, 14 years old or younger, 13 years old or younger, 12 years old or younger, 11 years old or younger, 10 years old or younger, 9 years old or younger, 8 years old or younger, 7 years old or younger, 6 years old or younger, 5 years old or younger, 4 years old or younger, 3 years old or younger, 2 years old or younger, or 1 year old or younger at the onset of the diabetes).

In some embodiments, the subject is or was determined to be 14 years old or younger at the onset of the diabetes (e.g., 15 years old or younger, 14 years old or younger, 13 years old or younger, 12 years old or younger, 11 years old or younger, 10 years old or younger, 9 years old or younger, 8 years old or younger, 7 years old or younger, 6 years old or younger, 5 years old or younger, 4 years old or younger, 3 years old or younger, 2 years old or younger, or 1 year old or younger at the onset of the diabetes).

In some embodiments, the subject is or was determined to be 13 years old or younger at the onset of the diabetes (e.g., 13 years old or younger, 12 years old or younger, 11 years old or younger, 10 years old or younger, 9 years old or younger, 8 years old or younger, 7 years old or younger, 6 years old or younger, 5 years old or younger, 4 years old or younger, 3 years old or younger, 2 years old or younger, or 1 year old or younger at the onset of the diabetes).

In some embodiments, the subject is or was determined to be 12 years old or younger at the onset of the diabetes (e.g., 12 years old or younger, 11 years old or younger, 10 years old or younger, 9 years old or younger, 8 years old or younger, 7 years old or younger, 6 years old or younger, 5 years old or younger, 4 years old or younger, 3 years old or younger, 2 years old or younger, or 1 year old or younger at the onset of the diabetes).

In some embodiments, the subject is or was determined to be 11 years old or younger at the onset of the diabetes (e.g., 11 years old or younger, 10 years old or younger, 9 years old or younger, 8 years old or younger, 7 years old or younger, 6 years old or younger, 5 years old or younger, 4 years old or younger, 3 years old or younger, 2 years old or younger, or 1 year old or younger at the onset of the diabetes).

In some embodiments, the subject is or was determined to be 10 years old or younger at the onset of the diabetes (e.g., 10 years old or younger, 9 years old or younger, 8 years old or younger, 7 years old or younger, 6 years old or younger, 5 years old or younger, 4 years old or younger, 3 years old or younger, 2 years old or younger, or 1 year old or younger at the onset of the diabetes).

In some embodiments, the subject is or was determined to be 9 years old or younger at the onset of the diabetes (e.g., 9 years old or younger, 8 years old or younger, 7 years old or younger, 6 years old or younger, 5 years old or younger, 4 years old or younger, 3 years old or younger, 2 years old or younger, or 1 year old or younger at the onset of the diabetes).

In some embodiments, the subject is or was determined to be 8 years old or younger at the onset of the diabetes (e.g., 8 years old or younger, 7 years old or younger, 6 years old or younger, 5 years old or younger, 4 years old or younger, 3 years old or younger, 2 years old or younger, or 1 year old or younger at the onset of the diabetes).

In some embodiments, the subject is or was determined to be 7 years old or younger at the onset of the diabetes (e.g., 7 years old or younger, 6 years old or younger, 5 years old or younger, 4 years old or younger, 3 years old or younger, 2 years old or younger, or 1 year old or younger at the onset of the diabetes).

In some embodiments, the subject is or was determined to be 6 years old or younger at the onset of the diabetes (e.g., 6 years old or younger, 5 years old or younger, 4 years old or younger, 3 years old or younger, 2 years old or younger, or 1 year old or younger at the onset of the diabetes). In some embodiments, the subject is or was determined to be 5 years old or younger at the onset of the diabetes (e.g., 5 years old or younger, 4 years old or younger, 3 years old or younger, 2 years old or younger, or 1 year old or younger at the onset of the diabetes).

In some embodiments, the subject is or was determined to be 4 years old or younger at the onset of the diabetes (e.g., 4 years old or younger, 3 years old or younger, 2 years old or younger, or 1 year old or younger at the onset of the diabetes).

In some embodiments, the subject is or was determined to be 3 years old or younger at the onset of the diabetes (e.g., 3 years old or younger, 2 years old or younger, or 1 year old or younger at the onset of the diabetes).

In some embodiments, the subject is or was determined to be 2 years old or younger at the onset of the diabetes (e.g., 2 years old or younger, or 1 year old or younger at the onset of the diabetes).

In some embodiments, is or was determined to be 1 year old or younger at the onset of the diabetes.

In some embodiments, the subject is or was determined to be about 1 year old to about 40 years old at the onset of the diabetes (e.g., about 1 year old to about 39 years old, about 1 year old to about 38 years old, about 1 year old to about 37 years old, about 1 year old to about 36 years old, about 1 year old to about 35 years old, about 1 year old to about 34 years old, about 1 year old to about 33 years old, about 1 year old to about 32 years old, about 1 year old to about 31 years old, about 1 year old to about 30 years old, about 1 year old to about 29 years old, about 1 year old to about 28 years old, about 1 year old to about 27 years old, about 1 year old to about 26 years old, about 1 year old to about 25 years old, about 1 year old to about 24 years old, about 1 year old to about 23 years old, about 1 year old to about 22 years old, about 1 year old to about 21 years old, about 1 year old to about 20 years old, about 1 year old to about 19 years old, about 1 year old to about 18 years old, about 1 year old to about 17 years old, about 1 year old to about 16 years old, about 1 year old to about 15 years old, about 1 year old to about 14 years old, about 1 year old to about 13 years old, about 1 year old to about 12 years old, about 1 year old to about 11 years old, about 1 year old to about 10 years old, about 1 year old to about 9 years old, about 1 year old to about 8 years old, about 1 year old to about 7 years old, about 1 year old to about 6 years old, about 1 year old to about 5 years old, about 1 year old to about 4 years old, about 1 year old to about 3 years old, or about 1 year old to about 2 years old at the onset of the diabetes).

In some embodiments, the subject is or was determined to be about 2 years old to about 40 years old at the onset of the diabetes (e.g., about 2 years old to about 39 years old, about 2 years old to about 38 years old, about 2 years old to about 37 years old, about 2 years old to about 36 years old, about 2 years old to about 35 years old, about 2 years old to about 34 years old, about 2 years old to about 33 years old, about 2 years old to about 32 years old, about 2 years old to about 31 years old, about 2 years old to about 30 years old, about 2 years old to about 29 years old, about 2 years old to about 28 years old, about 2 years old to about 27 years old, about 2 years old to about 26 years old, about 2 years old to about 25 years old, about 2 years old to about 24 years old, about 2 years old to about 23 years old, about 2 years old to about 22 years old, about 2 years old to about 21 years old, about 2 years old to about 20 years old, about 2 years old to about 19 years old, about 2 years old to about 18 years old, about 2 years old to about 17 years old, about 2 years old to about 16 years old, about 2 years old to about 15 years old, about 2 years old to about 14 years old, about 2 years old to about 13 years old, about 2 years old to about 12 years old, about 2 years old to about 11 years old, about 2 years old to about 10 years old, about 2 years old to about 9 years old, about 2 years old to about 8 years old, about 2 years old to about 7 years old, about 2 years old to about 6 years old, about 2 years old to about 5 years old, about 2 years old to about 4 years old, or about 2 years old to about 3 years old at the onset of the diabetes).

In some embodiments, the subject is or was determined to be about 3 years old to about 40 years old at the onset of the diabetes (e.g., about 3 years old to about 39 years old, about 3 years old to about 38 years old, about 3 years old to about 37 years old, about 3 years old to about 36 years old, about 3 years old to about 35 years old, about 3 years old to about 34 years old, about 3 years old to about 33 years old, about 3 years old to about 32 years old, about 3 years old to about 31 years old, about 3 years old to about 30 years old, about 3 years old to about 29 years old, about 3 years old to about 28 years old, about 3 years old to about 27 years old, about 3 years old to about 26 years old, about 3 years old to about 25 years old, about 3 years old to about 24 years old, about 3 years old to about 23 years old, about 3 years old to about 22 years old, about 3 years old to about 21 years old, about 3 years old to about 20 years old, about 3 years old to about 19 years old, about 3 years old to about 18 years old, about 3 years old to about 17 years old, about 3 years old to about 16 years old, about 3 years old to about 15 years old, about 3 years old to about 14 years old, about 3 years old to about 13 years old, about 3 years old to about 12 years old, about 3 years old to about 11 years old, about 3 years old to about 10 years old, about 3 years old to about 9 years old, about 3 years old to about 8 years old, about 3 years old to about 7 years old, about 3 years old to about 6 years old, about 3 years old to about 5 years old, or about 3 years old to about 4 years old at the onset of the diabetes).

In some embodiments, the subject is or was determined to be about 4 years old to about 40 years old at the onset of the diabetes (e.g., about 4 years old to about 39 years old, about 4 years old to about 38 years old, about 4 years old to about 37 years old, about 4 years old to about 36 years old, about 4 years old to about 35 years old, about 4 years old to about 34 years old, about 4 years old to about 33 years old, about 4 years old to about 32 years old, about 4 years old to about 31 years old, about 4 years old to about 30 years old, about 4 years old to about 29 years old, about 4 years old to about 28 years old, about 4 years old to about 27 years old, about 4 years old to about 26 years old, about 4 years old to about 25 years old, about 4 years old to about 24 years old, about 4 years old to about 23 years old, about 4 years old to about 22 years old, about 4 years old to about 21 years old, about 4 years old to about 20 years old, about 4 years old to about 19 years old, about 4 years old to about 18 years old, about 4 years old to about 17 years old, about 4 years old to about 16 years old, about 4 years old to about 15 years old, about 4 years old to about 14 years old, about 4 years old to about 13 years old, about 4 years old to about 12 years old, about 4 years old to about 11 years old, about 4 years old to about 10 years old, about 4 years old to about 9 years old, about 4 years old to about 8 years old, about 4 years old to about 7 years old, about 4 years old to about 6 years old, or about 4 years old to about 5 years old at the onset of the diabetes).

In some embodiments, the subject is or was determined to be about 5 years old to about 40 years old at the onset of the diabetes (e.g., about 5 years old to about 39 years old, about 5 years old to about 38 years old, about 5 years old to about 37 years old, about 5 years old to about 36 years old, about 5 years old to about 35 years old, about 5 years old to about 34 years old, about 5 years old to about 33 years old, about 5 years old to about 32 years old, about 5 years old to about 31 years old, about 5 years old to about 30 years old, about 5 years old to about 29 years old, about 5 years old to about 28 years old, about 5 years old to about 27 years old, about 5 years old to about 26 years old, about 5 years old to about 25 years old, about 5 years old to about 24 years old, about 5 years old to about 23 years old, about 5 years old to about 22 years old, about 5 years old to about 21 years old, about 5 years old to about 20 years old, about 5 years old to about 19 years old, about 5 years old to about 18 years old, about 5 years old to about 17 years old, about 5 years old to about 16 years old, about 5 years old to about 15 years old, about 5 years old to about 14 years old, about 5 years old to about 13 years old, about 5 years old to about 12 years old, about 5 years old to about 11 years old, about 5 years old to about 10 years old, about 5 years old to about 9 years old, about 5 years old to about 8 years old, about 5 years old to about 7 years old, or about 5 years old to about 6 years old at the onset of the diabetes).

In some embodiments, the subject has been determined to have first presented one or more symptoms of the diabetes at an age of less than 40 years (e.g., at an age of 39 years, 38 years, 37 years, 36 years, 35 years, 34 years, 33 years, 32 years, 31 years, 30 years, 29 years, 28 years, 27 years, 26 years, 25 years, 24 years, 23 years, 22 years, 21 years, 20 years, 19 years, 18 years, 17 years, 16 years, 15 years, 14 years, 13 years, 12 years, 11 years, 10 years, 9 years, 8 years, 7 years, 6 years, 5 years, 4 years, 3 years, 2 years, 1 year, or less.

In some embodiments, the subject has been determined to have first presented one or more symptoms of the diabetes at an age of from about 1 year to about 39 years (e.g., at an age of from about 1 year to about 38 years, at an age of from about 1 year to about 37 years, at an age of from about 1 year to about 36 years, at an age of from about 1 year to about 35 years, at an age of from about 1 year to about 34 years, at an age of from about 1 year to about 33 years, at an age of from about 1 year to about 32 years, at an age of from about 1 year to about 31 years, at an age of from about 1 year to about 30 years, at an age of from about 1 year to about 29 years, at an age of from about 1 year to about 28 years, at an age of from about 1 year to about 27 years, at an age of from about 1 year to about 26 years, at an age of from about 1 year to about 25 years, at an age of from about 1 year to about 24 years, at an age of from about 1 year to about 23 years, at an age of from about 1 year to about 22 years, at an age of from about 1 year to about 21 years, at an age of from about 1 year to about 20 years, at an age of from about 1 year to about 19 years, at an age of from about 1 year to about 18 years, at an age of from about 1 year to about 17 years, at an age of from about 1 year to about 16 years, at an age of from about 1 year to about 15 years, at an age of from about 1 year to about 14 years, at an age of from about 1 year to about 13 years, at an age of from about 1 year to about 12 years, at an age of from about 1 year to about 11 years, at an age of from about 1 year to about 10 years, at an age of from about 1 year to about 9 years, at an age of from about 1 year to about 8 years, at an age of from about 1 year to about 7 years, at an age of from about 1 year to about 6 years, at an age of from about 1 year to about 5 years, at an age of from about 1 year to about 4 years, at an age of from about 1 year to about 3 years, or at an age of from about 1 year to about 2 years).

In some embodiments, the subject has been determined to have first presented one or more symptoms of the diabetes at an age of from about 2 years to about 39 years (e.g., at an age of from about 2 years to about 38 years, at an age of from about 2 years to about 37 years, at an age of from about 2 years to about 36 years, at an age of from about 2 years to about 35 years, at an age of from about 2 years to about 34 years, at an age of from about 2 years to about 33 years, at an age of from about 2 years to about 32 years, at an age of from about 2 years to about 31 years, at an age of from about 2 years to about 30 years, at an age of from about 2 years to about 29 years, at an age of from about 2 years to about 28 years, at an age of from about 2 years to about 27 years, at an age of from about 2 years to about 26 years, at an age of from about 2 years to about 25 years, at an age of from about 2 years to about 24 years, at an age of from about 2 years to about 23 years, at an age of from about 2 years to about 22 years, at an age of from about 2 years to about 21 years, at an age of from about 2 years to about 20 years, at an age of from about 2 years to about 19 years, at an age of from about 2 years to about 18 years, at an age of from about 2 years to about 17 years, at an age of from about 2 years to about 16 years, at an age of from about 2 years to about 15 years, at an age of from about 2 years to about 14 years, at an age of from about 2 years to about 13 years, at an age of from about 2 years to about 12 years, at an age of from about 2 years to about 11 years, at an age of from about 2 years to about 10 years, at an age of from about 2 years to about 9 years, at an age of from about 2 years to about 8 years, at an age of from about 2 years to about 7 years, at an age of from about 2 years to about 6 years, at an age of from about 2 years to about 5 years, at an age of from about 2 years to about 4 years, or at an age of from about 2 years to about 3 years).

In some embodiments, the subject has been determined to have first presented one or more symptoms of the diabetes at an age of from about 3 years to about 39 years (e.g., at an age of from about 3 years to about 38 years, at an age of from about 3 years to about 37 years, at an age of from about 3 years to about 36 years, at an age of from about 3 years to about 35 years, at an age of from about 3 years to about 34 years, at an age of from about 3 years to about 33 years, at an age of from about 3 years to about 32 years, at an age of from about 3 years to about 31 years, at an age of from about 3 years to about 30 years, at an age of from about 3 years to about 29 years, at an age of from about 3 years to about 28 years, at an age of from about 3 years to about 27 years, at an age of from about 3 years to about 26 years, at an age of from about 3 years to about 25 years, at an age of from about 3 years to about 24 years, at an age of from about 3 years to about 23 years, at an age of from about 3 years to about 22 years, at an age of from about 3 years to about 21 years, at an age of from about 3 years to about 20 years, at an age of from about 3 years to about 19 years, at an age of from about 3 years to about 18 years, at an age of from about 3 years to about 17 years, at an age of from about 3 years to about 16 years, at an age of from about 3 years to about 15 years, at an age of from about 3 years to about 14 years, at an age of from about 3 years to about 13 years, at an age of from about 3 years to about 12 years, at an age of from about 3 years to about 11 years, at an age of from about 3 years to about 10 years, at an age of from about 3 years to about 9 years, at an age of from about 3 years to about 8 years, at an age of from about 3 years to about 7 years, at an age of from about 3 years to about 6 years, at an age of from about 3 years to about 5 years, or at an age of from about 3 years to about 4 years).

In some embodiments, the subject has been determined to have first presented one or more symptoms of the diabetes at an age of from about 3 years to about 39 years (e.g., at an age of from about 4 years to about 38 years, at an age of from about 4 years to about 37 years, at an age of from about 4 years to about 36 years, at an age of from about 4 years to about 35 years, at an age of from about 4 years to about 34 years, at an age of from about 4 years to about 33 years, at an age of from about 4 years to about 32 years, at an age of from about 4 years to about 31 years, at an age of from about 4 years to about 30 years, at an age of from about 4 years to about 29 years, at an age of from about 4 years to about 28 years, at an age of from about 4 years to about 27 years, at an age of from about 4 years to about 26 years, at an age of from about 4 years to about 25 years, at an age of from about 4 years to about 24 years, at an age of from about 4 years to about 23 years, at an age of from about 4 years to about 22 years, at an age of from about 4 years to about 21 years, at an age of from about 4 years to about 20 years, at an age of from about 4 years to about 19 years, at an age of from about 4 years to about 18 years, at an age of from about 4 years to about 17 years, at an age of from about 4 years to about 16 years, at an age of from about 4 years to about 15 years, at an age of from about 4 years to about 14 years, at an age of from about 4 years to about 13 years, at an age of from about 4 years to about 12 years, at an age of from about 4 years to about 11 years, at an age of from about 4 years to about 10 years, at an age of from about 4 years to about 9 years, at an age of from about 4 years to about 8 years, at an age of from about 4 years to about 7 years, at an age of from about 4 years to about 6 years, or at an age of from about 4 years to about 5 years).

In some embodiments, the subject has been determined to have first presented one or more symptoms of the diabetes at an age of from about 5 years to about 39 years (e.g., at an age of from about 5 years to about 38 years, at an age of from about 5 years to about 37 years, at an age of from about 5 years to about 36 years, at an age of from about 5 years to about 35 years, at an age of from about 5 years to about 34 years, at an age of from about 5 years to about 33 years, at an age of from about 5 years to about 32 years, at an age of from about 5 years to about 31 years, at an age of from about 5 years to about 30 years, at an age of from about 5 years to about 29 years, at an age of from about 5 years to about 28 years, at an age of from about 5 years to about 27 years, at an age of from about 5 years to about 26 years, at an age of from about 5 years to about 25 years, at an age of from about 5 years to about 24 years, at an age of from about 5 years to about 23 years, at an age of from about 5 years to about 22 years, at an age of from about 5 years to about 21 years, at an age of from about 5 years to about 20 years, at an age of from about 5 years to about 19 years, at an age of from about 5 years to about 18 years, at an age of from about 5 years to about 17 years, at an age of from about 5 years to about 16 years, at an age of from about 5 years to about 15 years, at an age of from about 5 years to about 14 years, at an age of from about 5 years to about 13 years, at an age of from about 5 years to about 12 years, at an age of from about 5 years to about 11 years, at an age of from about 5 years to about 10 years, at an age of from about 5 years to about 9 years, at an age of from about 5 years to about 8 years, at an age of from about 5 years to about 7 years, or at an age of from about 5 years to about 6 years).

In some embodiments, the one or more symptoms of the diabetes include:
a) a blood glucose concentration of about 200 mg/dL or greater (e.g., a blood glucose concentration of about 200 mg/dL, 201 mg/dL, 202 mg/dL, 203 mg/dL, 204 mg/dL, 205 mg/dL, 206 mg/dL, 207 mg/dL, 208 mg/dL, 209 mg/dL, 210 mg/dL, 211 mg/dL, 212 mg/dL, 213 mg/dL, 214 mg/dL, 215 mg/dL, 216 mg/dL, 217 mg/dL, 218 mg/dL, 219 mg/dL, 220 mg/dL, 221 mg/dL, 222 mg/dL, 223 mg/dL, 224 mg/dL, 225 mg/dL, 226 mg/dL, 227 mg/dL, 228 mg/dL, 229 mg/dL, 230 mg/dL, 231 mg/dL, 232 mg/dL, 233 mg/dL, 234 mg/dL, 235 mg/dL, 236 mg/dL, 237 mg/dL, 238 mg/dL, 239 mg/dL, 240 mg/dL, 241 mg/dL, 242 mg/dL, 243 mg/dL, 244 mg/dL, 245 mg/dL, 246 mg/dL, 247 mg/dL, 248 mg/dL, 249 mg/dL, 250 mg/dL, or greater);
b) a glycated hemoglobin (HbA1c) level of about 6.5% or greater (e.g., an HbA1c level of 6.5%, 6.6%, 6.7%, 6.8%, 6.9%, 7%, 7.1%, 7.2%, 7.3%, 7.4%, 7.5%, 7.6%, 7.7%, 7.8%, 7.9%, 8%, 8.1%, 8.2%, 8.3%, 8.4%, 8.5%, 8.6%, 8.7%, 8.8%, 8.9%, 9%, 9.1%, 9.2%, 9.3%, 9.4%, 9.5%, 9.6%, 9.7%, 9.8%, 9.9%, 10%, or greater);
c) a blood glucose concentration of about 100 mg/dL or greater (e.g., a blood glucose concentration of 100 mg/dL, 101 mg/dL, 102 mg/dL, 103 mg/dL, 104 mg/dL, 105 mg/dL, 106 mg/dL, 107 mg/dL, 108 mg/dL, 109 mg/dL, 110 mg/dL, 111 mg/dL, 112 mg/dL, 113 mg/dL, 114 mg/dL, 115 mg/dL, 116 mg/dL, 117 mg/dL, 118 mg/dL, 119 mg/dL, 120 mg/dL, 121 mg/dL, 122 mg/dL, 123 mg/dL, 124 mg/dL, 125 mg/dL, 126 mg/dL, 127 mg/dL, 128 mg/dL, 129 mg/dL, 130 mg/dL, 131 mg/dL, 132 mg/dL, 133 mg/dL, 134 mg/dL, 135 mg/dL, 136 mg/dL, 137 mg/dL, 138 mg/dL, 139 mg/dL, 140 mg/dL, 141 mg/dL, 142 mg/dL, 143 mg/dL, 144 mg/dL, 145 mg/dL, 146 mg/dL, 147 mg/dL, 148 mg/dL, 149 mg/dL, 150 mg/dL, or greater) in the subject in a fasting state, such as a fasting state in which the subject has not eaten for the previous 8-12 hours relative to the time at which the blood glucose concentration is measured; and/or
d) a presence of anti-glutamic acid decarboxylase (GAD) autoantibodies in peripheral blood isolated from the subject.

In some embodiments, the one or more symptoms of the diabetes include a blood glucose concentration of from about 100 mg/dL to about 125 mg/dL in the subject in a fasting state (e.g., a blood glucose concentration of 100 mg/dL, 101 mg/dL, 102 mg/dL, 103 mg/dL, 104 mg/dL, 105 mg/dL, 106 mg/dL, 107 mg/dL, 108 mg/dL, 109 mg/dL, 110 mg/dL, 111 mg/dL, 112 mg/dL, 113 mg/dL, 114 mg/dL, 115 mg/dL, 116 mg/dL, 117 mg/dL, 118 mg/dL, 119 mg/dL, 120 mg/dL, 121 mg/dL, 122 mg/dL, 123 mg/dL, 124 mg/dL, or 125 mg/dL in the subject in a fasting state). In some embodiments, the one or more symptoms of the diabetes include a blood glucose concentration of about 126 mg/dL or greater in the subject in a fasting state (e.g., a blood glucose concentration of 126 mg/dL, 127 mg/dL, 128 mg/dL, 129 mg/dL, 130 mg/dL, 131 mg/dL, 132 mg/dL, 133 mg/dL, 134 mg/dL, 135 mg/dL, 136 mg/dL, 137 mg/dL, 138 mg/dL, 139 mg/dL, 140 mg/dL, 141 mg/dL, 142 mg/dL, 143 mg/dL, 144 mg/dL, 145 mg/dL, 146 mg/dL, 147 mg/dL, 148 mg/dL, 149 mg/dL, 150 mg/dL, 200 mg/dL, or greater in the subject in a fasting state).

In some embodiments, the subject was less than 40 years old at the time of diagnosis of the diabetes in the subject (e.g., 40 years old, 39 years old, 38 years old, 37 years old, 36 years old, 35 years old, 34 years old, 33 years old, 32 years old, 31 years old, 30 years old, 29 years old, 28 years old, 27 years old, 26 years old, 25 years old, 24 years old, 23 years old, 22 years old, 21 years old, 20 years old, 19 years old, 18 years old, 17 years old, 16 years old, 15 years old, 14 years old, 13 years old, 12 years old, 11 years old, 10 years old, 9 years old, 8 years old, 7 years old, 6 years old, 5 years old, 4 years old, 3 years old, 2 years old, 1 year old, or younger).

In some embodiments, the subject was from about 1 year old to about 39 years old at the time of diagnosis of the diabetes in the subject (e.g., from about 1 year old to about 38 years old, from about 1 year old to about 37 years old, from about 1 year old to about 36 years old, from about 1 year old to about 35 years old, from about 1 year old to about 34 years old, from about 1 year old to about 33 years old, from about 1 year old to about 32 years old, from about 1 year old to about 31 years old, from about 1 year old to about 30 years old, from about 1 year old to about 29 years old, from about 1 year old to about 28 years old, from about 1 year old to about 27 years old, from about 1 year old to about 26 years old, from about 1 year old to about 25 years old, from about 1 year old to about 24 years old, from about 1 year old to about 23 years old, from about 1 year old to about 22 years old, from about 1 year old to about 21 years old, from about 1 year old to about 20 years old, from about 1 year old to about 19 years old, from about 1 year old to about 18 years old, from about 1 year old to about 17 years old, from about 1 year old to about 16 years old, from about 1 year old to about 15 years old, from about 1 year old to about 14 years old, from about 1 year old to about 13 years old, from about 1 year old to about 12 years old, from about 1 year old to about 11 years old, from about 1 year old to about 10 years old, from about 1 year old to about 9 years old, from about 1 year old to about 8 years old, from about 1 year old to about 7 years old, from about 1 year old to about 6 years old, from about 1 year old to about 5 years old, from about 1 year old to about 4 years old, from about 1 year old to about 3 years old, or from about 1 year old to about 2 years old).

In some embodiments, the subject was from about 2 years old to about 39 years old at the time of diagnosis of the diabetes in the subject (e.g., from about 2 years old to about 38 years old, from about 2 years old to about 37 years old, from about 2 years old to about 36 years old, from about 2 years old to about 35 years old, from about 2 years old to about 34 years old, from about 2 years old to about 33 years old, from about 2 years old to about 32 years old, from about 2 years old to about 31 years old, from about 2 years old to about 30 years old, from about 2 years old to about 29 years old, from about 2 years old to about 28 years old, from about 2 years old to about 27 years old, from about 2 years old to about 26 years old, from about 2 years old to about 25 years old, from about 2 years old to about 24 years old, from about 2 years old to about 23 years old, from about 2 years old to about 22 years old, from about 2 years old to about 21 years old, from about 2 years old to about 20 years old, from about 2 years old to about 19 years old, from about 2 years old to about 18 years old, from about 2 years old to about 17 years old, from about 2 years old to about 16 years old, from about 2 years old to about 15 years old, from about 2 years old to about 14 years old, from about 2 years old to about 13 years old, from about 2 years old to about 12 years old, from about 2 years old to about 11 years old, from about 2 years old to about 10 years old, from about 2 years old to about 9 years old, from about 2 years old to about 8 years old, from about 2 years old to about 7 years old, from about 2 years old to about 6 years old, from about 2 years old to about 5 years old, from about 2 years old to about 4 years old, or from about 2 years old to about 3 years old).

In some embodiments, the subject was from about 3 years old to about 39 years old at the time of diagnosis of the diabetes in the subject (e.g., from about 3 years old to about 38 years old, from about 3 years old to about 37 years old, from about 3 years old to about 36 years old, from about 3 years old to about 35 years old, from about 3 years old to about 34 years old, from about 3 years old to about 33 years old, from about 3 years old to about 32 years old, from about 3 years old to about 31 years old, from about 3 years old to about 30 years old, from about 3 years old to about 29 years old, from about 3 years old to about 28 years old, from about 3 years old to about 27 years old, from about 3 years old to about 26 years old, from about 3 years old to about 25 years old, from about 3 years old to about 24 years old, from about 3 years old to about 23 years old, from about 3 years old to about 22 years old, from about 3 years old to about 21 years old, from about 3 years old to about 20 years old, from about 3 years old to about 19 years old, from about 3 years old to about 18 years old, from about 3 years old to about 17 years old, from about 3 years old to about 16 years old, from about 3 years old to about 15 years old, from about 3 years old to about 14 years old, from about 3 years old to about 13 years old, from about 3 years old to about 12 years old, from about 3 years old to about 11 years old, from about 3 years old to about 10 years old, from about 3 years old to about 9 years old, from about 3 years old to about 8 years old, from about 3 years old to about 7 years old, from about 3 years old to about 6 years old, from about 3 years old to about 5 years old, or from about 3 years old to about 4 years old).

In some embodiments, the subject was from about 4 years old to about 39 years old at the time of diagnosis of the diabetes in the subject (e.g., from about 4 years old to about 38 years old, from about 4 years old to about 37 years old, from about 4 years old to about 36 years old, from about 4 years old to about 35 years old, from about 4 years old to about 34 years old, from about 4 years old to about 33 years old, from about 4 years old to about 32 years old, from about 4 years old to about 31 years old, from about 4 years old to about 30 years old, from about 4 years old to about 29 years old, from about 4 years old to about 28 years old, from about 4 years old to about 27 years old, from about 4 years old to about 26 years old, from about 4 years old to about 25 years old, from about 4 years old to about 24 years old, from about 4 years old to about 23 years old, from about 4 years old to about 22 years old, from about 4 years old to about 21 years old, from about 4 years old to about 20 years old, from about 4 years old to about 19 years old, from about 4 years old to about 18 years old, from about 4 years old to about 17 years old, from about 4 years old to about 16 years old, from about 4 years old to about 15 years old, from about 4 years old to about 14 years old, from about 4 years old to about 13 years old, from about 4 years old to about 12 years old, from about 4 years old to about 11 years old, from about 4 years old to about 10 years old, from about 4 years old to about 9 years old, from about 4 years old to about 8 years old, from about 4 years old to about 7 years old, from about 4 years old to about 6 years old, or from about 4 years old to about 5 years old).

In some embodiments, the subject was from about 5 years old to about 39 years old at the time of diagnosis of the diabetes in the subject (e.g., from about 5 years old to about 38 years old, from about 5 years old to about 37 years old, from about 5 years old to about 36 years old, from about 5 years old to about 35 years old, from about 5 years old to about 34 years old, from about 5 years old to about 33 years old, from about 5 years old to about 32 years old, from about 5 years old to about 31 years old, from about 5 years old to about 30 years old, from about 5 years old to about 29 years old, from about 5 years old to about 28 years old, from about 5 years old to about 27 years old, from about 5 years old to about 26 years old, from about 5 years old to about 25 years old, from about 5 years old to about 24 years old, from about 5 years old to about 23 years old, from about 5 years old to about 22 years old, from about 5 years old to about 21 years old, from about 5 years old to about 20 years old, from about 5 years old to about 19 years old, from about 5 years old to about 18 years old, from about 5 years old to about 17 years old, from about 5 years old to about 16 years old, from about 5 years old to about 15 years old, from about 5 years old to about 14 years old, from about 5 years old to about 13 years old, from about 5 years old to about 12 years old, from about 5 years old to about 11 years old, from about 5 years old to about 10 years old, from about 5 years old to about 9 years old, from about 5 years old to about 8 years old, from about 5 years old to about 7 years old, or from about 5 years old to about 6 years old).

In some embodiments of any of the foregoing aspects or embodiments of the disclosure, the subject has been diagnosed as having juvenile onset diabetes.

In some embodiments of any of the foregoing aspects or embodiments of the disclosure, prior to administration of the BCG to the subject, the subject has been determined to exhibit a rate of glucose uptake that is less than a reference rate of glucose uptake (e.g., a rate of glucose uptake that is less than a reference rate of glucose uptake (e.g., a reference rate of from about 6,000 MFI to about 10,000 MFI as assessed by way of a fluorescently-labeled glucose uptake assay described herein) by 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, or more). In some embodiments, prior to administration of the BCG to the subject, the subject has been determined to exhibit a rate of oxidative phosphorylation that is greater than a reference rate of oxidative phosphorylation (e.g., a rate of oxidative phosphorylation that is greater than a reference rate of oxidative phosphorylation (e.g., a reference rate of from about 25 pmol of O₂ consumption per minute to about 150 pmol of O₂ consumption per minute) by 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 200%, 300%, 400%, 500%, or more). In some embodiments, prior to administration of the BCG to the subject, the subject has been determined to exhibit a rate of aerobic glycolysis that is less than a reference rate of aerobic glycolysis (e.g., a rate of aerobic glycolysis that is less than a reference rate of aerobic glycolysis (e.g., a reference rate of from about 5 mpH units per minute to about 50 mpH units per minute) by 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, or more).

In some embodiments, the rate of glucose uptake exhibited by the subject is assessed by evaluating the rate of glucose uptake in a sample of cells obtained from the subject. The cells may include, for example, peripheral blood cells, such as leukocytes (e.g., monocytes, neutrophils, basophils, eosinophils, and/or lymphocytes, such as CD4+ lymphocytes). In some embodiments, the cells obtained from the subject include monocytes.

In some embodiments, the rate of glucose uptake in the sample of cells obtained from the subject is evaluated by a method that includes:
a) contacting the cells with a compound containing glucose covalently bound to a fluorophore;
b) washing the cells to remove any such compound that has not been internalized by the cells; and
c) measuring the fluorescence of the cells at a wavelength corresponding to an emission wavelength of the fluorophore,
   wherein intensity of fluorescence exhibited by the cells at the emission wavelength of the fluorophore correlates with uptake of glucose by the cells.

In some embodiments, the fluorophore includes a moiety selected from 7-nitrobenz-2-oxa-1,3-diazol-4-yl (NBD), methoxycoumarin, dialkylaminocoumarin, hydroxycoumarin, aminomethylcoumarin acetate (AMCA), 6,8-difluoro-7-hydroxy-3-carboxycoumarin (PACIFIC BLUE™ dye), 5-(dimethylamino)naphthalene-1-sulfonyl (Dansyl), pyrene, 7-amino-3-{[(2,5-dioxopyrrolidin-1-yl)oxy]-2-oxoethyl}-4-methyl-2-oxo-2H-chromene-6-sulfonic acid (ALEXA FLUOR 350™), 6,8-difluoro-7-hydroxy-4-methylcoumarin (MARINA BLUE™ dye), N-(2-aminoethyl)-4-{5-[4-(dimethylamino)phenyl]-1,3-oxazol-2-yl}benzenesulfonamide (DAPOXYL™ dye), 2,3,5,6-Tetramethyl-1H,7H-pyrazolo[1,2-a]pyrazole-1,7-dione (Bimane), 4-{[4-(Diethylamino)phenyl][4-(ethylamino)naphthalen-2-yl]methylidene}-N,N-diethylcyclohexa-2,5-dien-1-iminium (CASCADE BLUE™ dye), tris(N,N-diethylethanaminium) 8-[2-(4-{[(2,5-dioxopyrrolidin-1-yl)oxy]carbonyl}piperidin-1-yl)-2-oxoethoxy]pyrene-1,3,6-trisulfonate (ALEXA FLUOR 405™), N,N-diethylethanaminium [9-{6-[(2,5-dioxopyrrolidin-1-yl)oxy]-6-oxohexyl}-8,8-dimethyl-2-oxo-4-(trifluoromethyl)-8,9-dihydro-2H-benzo[g]chromen-6-yl]methanesulfonate (ALEXA FLUOR 430™), 1-[({4-[(7-nitro-2,1,3-benzoxadiazol-4-yl)amino]phenyl}acetyl)oxy]pyrrolidine-2,5-dione (QSY™ dye), fluorescein, 2-(6-amino-3-iminio-4,5-disulfonato-3H-xanthen-9-yl)-5-((2,3,5,6-tetrafluorophenoxy)carbonyl)benzoate (ALEXA FLUOR 488™), 2′,7′-Difluoro-3′,6′-dihydroxy-3H-spiro[isobenzofuran-1,9′-xanthen]-3-one (OREGON GREEN™ 488), 1,3,5,7,8-pentamethyl-4,4-difluorro-4-bora-3a,4a-diaza-s-indacene (BODIPY™ 493/503), rhodamine green, and rhodamine red. For example, in some embodiments, the compound is 2-[N-(7-nitrobenz-2-oxa-1,3-diazol-4-yl)amino]-2-deoxy-D-glucose (2-NBDG).

In some embodiments, the reference rate of glucose uptake is a rate of glucose uptake exhibited by a reference subject (e.g., a mammalian subject, such as a human subject) that does not have diabetes (e.g., a reference rate of from about 6,000 MFI to about 10,000 MFI as assessed by way of a fluorescently-labeled glucose uptake assay described herein). The reference rate of glucose uptake may be assessed, for example, by evaluating the rate of glucose uptake in a sample of cells obtained from the reference subject. In some embodiments, the cells obtained from the reference subject include peripheral blood cells, such as leukocytes (e.g., monocytes, neutrophils, basophils, eosinophils, and/or lymphocytes, such as CD4+ lymphocytes). In some embodiments, the cells obtained from the reference subject include monocytes.

In some embodiments, the rate of glucose uptake in the sample of cells obtained from the reference subject is evaluated by a method that includes:
a) contacting the cells with a compound containing glucose covalently bound to a fluorophore;
b) washing the cells to remove any such compound that has not been internalized by the cells; and
c) measuring the fluorescence of the cells at a wavelength corresponding to an emission wavelength of the fluorophore,
   wherein intensity of fluorescence exhibited by the cells at the emission wavelength of the fluorophore correlates with uptake of glucose by the cells. The fluorophore may include, for example, a moiety selected from 7-nitrobenz-2-oxa-1,3-diazol-4-yl (NBD), methoxycoumarin, dialkylaminocoumarin, hydroxycoumarin, aminomethylcoumarin acetate (AMCA), 6,8-difluoro-7-hydroxy-3-carboxycoumarin (PACIFIC BLUE™ dye), 5-(dimethylamino)naphthalene-1-sulfonyl (Dansyl), pyrene, 7-amino-3-{[(2,5-dioxopyrrolidin-1-yl)oxy]-2-oxoethyl}-4-methyl-2-oxo-2H-chromene-6-sulfonic acid (ALEXA FLUOR 350™), 6,8-difluoro-7-hydroxy-4-methylcoumarin (MARINA BLUE™ dye), N-(2-aminoethyl)-4-{5-[4-(dimethylamino)phenyl]-1,3-oxazol-2-yl}benzenesulfonamide (DAPOXYL™ dye), 2,3,5,6-Tetramethyl-1H,7H-pyrazolo[1,2-a]pyrazole-1,7-dione (Bimane), 4-{[4-(Diethylamino)phenyl][4-(ethylamino)naphthalen-2-yl]methylidene}-N,N-diethylcyclohexa-2,5-dien-1-iminium (CASCADE BLUE™ dye), tris(N,N-diethylethanaminium) 8-[2-(4-{[(2,5-dioxopyrrolidin-1-yl)oxy]carbonyl}piperidin-1-yl)-2-oxoethoxy]pyrene-1,3,6-trisulfonate (ALEXA FLUOR 405™), N,N-diethylethanaminium [9-{6-[(2,5-dioxopyrrolidin-1-yl)oxy]-6-oxohexyl}-8,8-dimethyl-2-oxo-4-(trifluoromethyl)-8,9-dihydro-2H-benzo[g]chromen-6-yl]methanesulfonate (ALEXA FLUOR 430™), 1-[({4-[(7-nitro-2,1,3-benzoxadiazol-4-yl)amino]phenyl}acetyl)oxy]pyrrolidine-2,5-dione (QSY™ dye), fluorescein, 2-(6-amino-3-iminio-4,5-disulfonato-3H-xanthen-9-yl)-5-((2,3,5,6-tetrafluorophenoxy)carbonyl)benzoate (ALEXA FLUOR 488™) 2′,7′-Difluoro-3′,6′-dihydroxy-3H-spiro[isobenzofuran-1,9′-xanthen]-3-one (OREGON GREEN™ 488), 1,3,5,7,8-pentamethyl-4,4-difluorro-4-bora-3a,4a-diaza-s-indacene (BODIPY™ 493/503), rhodamine green, and rhodamine red. In some embodiments, the compound is 2-[N-(7-nitrobenz-2-oxa-1,3-diazol-4-yl)amino]-2-deoxy-D-glucose (2-NBDG).

In some embodiments, the rate of oxidative phosphorylation exhibited by the subject is assessed by evaluating the rate of oxygen consumption in a sample of cells obtained from the subject. The cells may include, for example, peripheral blood cells, such as leukocytes (e.g., monocytes, neutrophils, basophils, eosinophils, and/or lymphocytes, such as CD4+ lymphocytes). In some embodiments, the cells obtained from the subject include monocytes.

In some embodiments, the rate of aerobic glycolysis exhibited by the subject is assessed by evaluating the rate of extracellular acidification in a sample of cells obtained from the subject. The cells may include, for example, peripheral blood cells, such as leukocytes (e.g., monocytes, neutrophils, basophils, eosinophils, and/or lymphocytes, such as CD4+ lymphocytes). In some embodiments, the cells obtained from the subject include monocytes.

In some embodiments, the reference rate of oxidative phosphorylation is a rate of oxidative phosphorylation exhibited by a reference subject (e.g., a mammalian subject, such as a human subject) that does not have diabetes. The reference rate of oxidative phosphorylation may be assessed, for example, by evaluating the rate of oxygen consumption in a sample of cells obtained from the reference subject. The cells may include, for example, peripheral blood cells, such as leukocytes (e.g., monocytes, neutrophils, basophils, eosinophils, and/or lymphocytes, such as CD4+ lymphocytes). In some embodiments, the cells obtained from the reference subject include monocytes.

In some embodiments, the reference rate of aerobic glycolysis is a rate of aerobic glycolysis exhibited by a reference subject (e.g., a mammalian subject, such as a human subject) that does not have diabetes. The reference rate of aerobic glycolysis may be assessed, for example, by evaluating the rate of extracellular acidification in a sample of cells obtained from the reference subject. The cells may include, for example, peripheral blood cells, such as leukocytes (e.g., monocytes, neutrophils, basophils, eosinophils, and/or lymphocytes, such as CD4+ lymphocytes). In some embodiments, the cells obtained from the reference subject include monocytes.

In some embodiments, the first and/or second sample of cells obtained from the subject prior to administration of BCG include peripheral blood cells, such as leukocytes (e.g., monocytes, neutrophils, basophils, eosinophils, and/or lymphocytes, such as CD4+ lymphocytes). In some embodiments, the first and/or second sample of cells obtained from the subject prior to administration of BCG include monocytes.

In some embodiments, the second sample of cells is cultured with BCG for from about 1 hour to about 1 week, or more (e.g., for about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 25 hours, 26 hours, 27 hours, 28 hours, 29 hours, 30 hours, 31 hours, 32 hours, 33 hours, 34 hours, 35 hours, 36 hours, 37 hours, 38 hours, 39 hours, 40 hours, 41 hours, 42 hours, 43 hours, 44 hours, 45 hours, 46 hours, 47 hours, 48 hours, 49 hours, 50 hours, 51 hours, 52 hours, 53 hours, 54 hours, 55 hours, 56 hours, 57 hours, 58 hours, 59 hours, 60 hours, 61 hours, 62 hours, 63 hours, 64 hours, 65 hours, 66 hours, 67 hours, 68 hours, 69 hours, 70 hours, 71 hours, or 72 hours, or more). In some embodiments, the second sample of cells is cultured with the BCG for from about 12 hours to about 48 hours (e.g., for about 12 hours, 13 hours, 14 hours, 15 hours, 16 hours, 17 hours, 18 hours, 19 hours, 20 hours, 21 hours, 22 hours, 23 hours, 24 hours, 25 hours, 26 hours, 27 hours, 28 hours, 29 hours, 30 hours, 31 hours, 32 hours, 33 hours, 34 hours, 35 hours, 36 hours, 37 hours, 38 hours, 39 hours, 40 hours, 41 hours, 42 hours, 43 hours, 44 hours, 45 hours, 46 hours, 47 hours, or 48 hours). In some embodiments, the second sample of cells is cultured with the BCG for from about 18 hours to about 36 hours (e.g., for about 18 hours, 19 hours, 20 hours, 21 hours, 22 hours, 23 hours, 24 hours, 25 hours, 26 hours, 27 hours, 28 hours, 29 hours, 30 hours, 31 hours, 32 hours, 33 hours, 34 hours, 35 hours, or 36 hours). In some embodiments, the second sample of cells is cultured with the BCG for about 24 hours.

In some embodiments, the rate of glucose uptake in the first and second samples of cells obtained from the subject prior to administration of BCG is evaluated by a method that includes:
a) contacting the first and second samples of cells with a compound containing glucose covalently bound to a fluorophore;
b) washing the first and second samples of cells to remove any such compound that has not been internalized by the cells; and
c) measuring the fluorescence of the first and second samples of cells at a wavelength corresponding to an emission wavelength of the fluorophore,
   wherein intensity of fluorescence exhibited by the first and second samples of cells at the emission wavelength of the fluorophore correlates with uptake of glucose by the first and second samples of cells. The fluorophore may contain, for example, a moiety selected from 7-nitrobenz-2-oxa-1,3-diazol-4-yl (NBD), methoxycoumarin, dialkylaminocoumarin, hydroxycoumarin, aminomethylcoumarin acetate (AMCA), 6,8-difluoro-7-hydroxy-3-carboxycoumarin (PACIFIC BLUE™ dye), 5-(dimethylamino)naphthalene-1-sulfonyl (Dansyl), pyrene, 7-amino-3-{[(2,5-dioxopyrrolidin-1-yl)oxy]-2-oxoethyl}-4-methyl-2-oxo-2H-chromene-6-sulfonic acid (ALEXA FLUOR 350™), 6,8-difluoro-7-hydroxy-4-methylcoumarin (MARINA BLUE™ dye), N-(2-aminoethyl)-4-{5-[4-(dimethylamino)phenyl]-1,3-oxazol-2-yl}benzenesulfonamide (DAPOXYL™ dye), 2,3,5,6-Tetramethyl-1H,7H-pyrazolo[1,2-a]pyrazole-1,7-dione (Bimane), 4-{[4-(Diethylamino)phenyl][4-(ethylamino)naphthalen-2-yl]methylidene}-N,N-diethylcyclohexa-2,5-dien-1-iminium (CASCADE BLUE™ dye), tris(N,N-diethylethanaminium) 8-[2-(4-{[(2,5-dioxopyrrolidin-1-yl)oxy]carbonyl}piperidin-1-yl)-2-oxoethoxy]pyrene-1,3,6-trisulfonate (ALEXA FLUOR 405™), N,N-diethylethanaminium [9-{6-[(2,5-dioxopyrrolidin-1-yl)oxy]-6-oxohexyl}-8,8-dimethyl-2-oxo-4-(trifluoromethyl)-8,9-dihydro-2H-benzo[g]chromen-6-yl]methanesulfonate (ALEXA FLUOR 430™), 1-[({4-[(7-nitro-2,1,3-benzoxadiazol-4-yl)amino]phenyl}acetyl)oxy]pyrrolidine-2,5-dione (QSY™ dye), fluorescein, 2-(6-amino-3-iminio-4,5-disulfonato-3H-xanthen-9-yl)-5-((2,3,5,6-tetrafluorophenoxy)carbonyl)benzoate (ALEXA FLUOR 488™), 2′,7′-Difluoro-3′,6′-dihydroxy-3H-spiro[isobenzofuran-1,9′-xanthen]-3-one (OREGON GREEN™ 488), 1,3,5,7,8-pentamethyl-4,4-difluorro-4-bora-3a,4a-diaza-s-indacene (BODIPY™ 493/503), rhodamine green, and rhodamine red. In some embodiments, the compound is 2-[N-(7-nitrobenz-2-oxa-1,3-diazol-4-yl)amino]-2-deoxy-D-glucose.

In some embodiments, the rate of oxidative phosphorylation in the first and second samples of cells obtained from the subject prior to administration of BCG is evaluated by measuring the rate of oxygen consumption in the first and second samples of cells.

In some embodiments, the rate of aerobic glycolysis in the first and second samples of cells obtained from the subject prior to administration of BCG is evaluated by measuring the rate of extracellular acidification in the first and second samples of cells.

In some embodiments of any of the foregoing aspects or embodiments of the disclosure, the BCG is administered to the subject in a single dose. In some embodiments, the BCG is administered to the subject in multiple doses.

For example, the BCG may be administered to the subject in one or more doses per day, week, month, or year, such as in from 1 to 10 doses per day, week, month, or year. In some embodiments, the BCG is administered to the subject in one or more doses per year, such as in from 1 to 10 doses per year (e.g., in 1, 2, 3, 4, 5, 6, 7, 8, 9, or 10 doses per year). In some embodiments, the BCG Is administered to the subject in from 1 to 5 doses per year (e.g., in 1, 2, 3, 4, or 5 doses per year).

In some embodiments, the BCG is administered to the subject during a first and second, or subsequent, treatment period, in which the treatment periods have a frequency of no greater than once per year (e.g., no greater than once every 1, 2, 3, 4, 5, 6, or more years), and in which during each treatment period the BCG is administered to the subject in one or more doses per treatment period, and the BCG is not administered to the subject during the time elapsed between the treatment periods.

In some embodiments, the BCG is administered to the subject in a total of from 2 to 20 doses (e.g., in 2, 3, 4, 5, 6, 7, 8, 9, 10, 11, 12, 13, 14, 15, 16, 17, 18, 19, or 20 doses). In some embodiments, the BCG is administered to the subject in a total of from 2 to 10 doses (e.g., in 2, 3, 4, 5, 6, 7, 8, 9, or 10 doses). In some embodiments, the BCG is administered to the subject in a total of from 2 to 5 doses (e.g., in 2, 3, 4, or 5 doses).

In some embodiments, a time elapsed from administration of one dose of the BCG to administration of a subsequent dose of the BCG is from about 1 day to about 1 year (e.g., about 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, or 52 weeks). In some embodiments, the time elapsed from administration of one dose of the BCG to administration of another dose of the BCG is from about 1 week to about 12 weeks (e.g., about 1 week, 2 weeks, 3 weeks, 4 weeks, 5 weeks, 6 weeks, 7 weeks, 8 weeks, 9 weeks, 10 weeks, 11 weeks, or 12 weeks). In some embodiments, a time elapsed from administration of one dose of the BCG to administration of a subsequent dose of the BCG is from about 2 weeks to about 8 weeks (e.g., about 2 weeks, 3 weeks, 4 weeks, 5 weeks, 6 weeks, 7 weeks, or 8 weeks). In some embodiments, a time elapsed from administration of one dose of the BCG to administration of a subsequent dose of the BCG is from about 3 weeks to about 6 weeks (e.g., about 3 weeks, 4 weeks, 5 weeks, or 6 weeks). In some embodiments, a time elapsed from administration of one dose of the BCG to administration of a subsequent dose of the BCG is about 4 weeks.

In some embodiments, a time elapsed from administration of one dose of the BCG to administration of a subsequent dose of the BCG is from about 2 months to about 72 months (e.g., about 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, 36 months, 37 months, 38 months, 39 months, 40 months, 41 months, 42 months, 43 months, 44 months, 45 months, 46 months, 47 months, 48 months, 49 months, 50 months, 51 months, 52 months, 53 months, 54 months, 55 months, 56 months, 57 months, 58 months, 59 months, 60 months, 61 months, 62 months, 63 months, 64 months, 65 months, 66 months, 67 months, 68 months, 69 months, 70 months, 71 months, or 72 months). In some embodiments, a time elapsed from administration of one dose of the BCG to administration of a subsequent dose of the BCG is from about 3 months to about 48 months (e.g., about 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, 36 months, 37 months, 38 months, 39 months, 40 months, 41 months, 42 months, 43 months, 44 months, 45 months, 46 months, 47 months, or 48 months). In some embodiments, a time elapsed from administration of one dose of the BCG to administration of a subsequent dose of the BCG is from about 6 months to about 36 months (e.g., about 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, or 36 months). In some embodiments, a time elapsed from administration of one dose of the BCG to administration of a subsequent dose of the BCG is from about 8 months to about 16 months (e.g., about 8 months, 9 months, 10 months, 11 months, 12 months, 13 months, 14 months, 15 months, or 16 months). In some embodiments, a time elapsed from administration of one dose of the BCG to administration of a subsequent dose of the BCG is about 12 months.

In some embodiments of any of the foregoing aspects or embodiments of the disclosure, the BCG is administered to the subject in at least first, second, and third doses. In some embodiments:
a) the second dose of the BCG is administered to the subject from about 1 day to about 1 year after administration of the first dose of the BCG to the subject; and
b) the third dose of the BCG is administered to the subject from about 2 months to about 72 months after administration of the second dose of the BCG to the subject.

In some embodiments, the second dose of the BCG is administered to the subject from about 1 week to about 12 weeks after administration of the first dose of the BCG to the subject (e.g., from about 2 weeks to about 8 weeks after administration of the first dose of the BCG to the subject, from about 3 weeks to about 6 weeks after administration of the first dose of the BCG to the subject, or about 4 weeks after administration of the first dose of the BCG to the subject).

In some embodiments, the third dose of the BCG is administered to the subject from about 3 months to about 48 months after administration of the second dose of the BCG to the subject (e.g., from about 6 months to about 36 months after administration of the second dose of the BCG to the subject, from about 8 months to about 16 months after administration of the second dose of the BCG to the subject, or about 12 months after administration of the second dose of the BCG to the subject).

In some embodiments, the BCG is additionally administered to the subject in at least fourth, fifth, and sixth doses. In some embodiments:
a) the fourth dose of the BCG is administered to the subject from about 2 months to about 72 months after administration of the third dose of the BCG to the subject;
b) the fifth dose of the BCG is administered to the subject from about 2 months to about 72 months after administration of the fourth dose of the BCG to the subject; and
c) the sixth dose of the BCG is administered to the subject from about 2 months to about 72 months after administration of the fifth dose of the BCG to the subject.

In some embodiments, the fourth dose of the BCG is administered to the subject from about 3 months to about 48 months after administration of the third dose of the BCG to the subject (e.g., from about 6 months to about 36 months after administration of the third dose of the BCG to the subject, from about 8 months to about 16 months after administration of the third dose of the BCG to the subject, or about 12 months after administration of the third dose of the BCG to the subject).

In some embodiments, the fifth dose of the BCG is administered to the subject from about 3 months to about 48 months after administration of the fourth dose of the BCG to the subject (e.g., from about 6 months to about 36 months after administration of the fourth dose of the BCG to the subject, from about 8 months to about 16 months after administration of the fourth dose of the BCG to the subject, or about 12 months after administration of the fourth dose of the BCG to the subject).

In some embodiments, the sixth dose of the BCG is administered to the subject from about 3 months to about 48 months after administration of the fifth dose of the BCG to the subject (e.g., from about 6 months to about 36 months after administration of the fifth dose of the BCG to the subject, from about 8 months to about 16 months after administration of the fifth dose of the BCG to the subject, or about 12 months after administration of the fifth dose of the BCG to the subject).

In some embodiments, each dose of BCG comprises from about 1×10⁴ colony forming units (CFU) per 0.1 mg of BCG to about 1×10⁸ CFU per 0.1 mg of BCG (e.g., about 1×10⁴ CFU per 0.1 mg of BCG, 2×10⁴ CFU per 0.1 mg of BCG, 3×10⁴ CFU per 0.1 mg of BCG, 4×10⁴ CFU per 0.1 mg of BCG, 5×10⁴ CFU per 0.1 mg of BCG, 6×10⁴ CFU per 0.1 mg of BCG, 7×10⁴ CFU per 0.1 mg of BCG, 8×10⁴ CFU per 0.1 mg of BCG, 9×10⁴ CFU per 0.1 mg of BCG, 1×10⁵ CFU per 0.1 mg of BCG, 2×10⁵ CFU per 0.1 mg of BCG, 3×10⁵ CFU per 0.1 mg of BCG, 4×10⁵ CFU per 0.1 mg of BCG, 5×10⁵ CFU per 0.1 mg of BCG, 6×10⁵ CFU per 0.1 mg of BCG, 7×10⁵ CFU per 0.1 mg of BCG, 8×10⁵ CFU per 0.1 mg of BCG, 9×10⁵ CFU per 0.1 mg of BCG, 1×10⁶ CFU per 0.1 mg of BCG, 2×10⁶ CFU per 0.1 mg of BCG, 3×10⁶ CFU per 0.1 mg of BCG, 4×10⁶ CFU per 0.1 mg of BCG, 5×10⁶ CFU per 0.1 mg of BCG, 6×10⁶ CFU per 0.1 mg of BCG, 7×10⁶ CFU per 0.1 mg of BCG, 8×10⁶ CFU per 0.1 mg of BCG, 9×10⁶ CFU per 0.1 mg of BCG, 1×10⁷ CFU per 0.1 mg of BCG, 2×10⁷ CFU per 0.1 mg of BCG, 3×10⁷ CFU per 0.1 mg of BCG, 4×10⁷ CFU per 0.1 mg of BCG, 5×10⁷ CFU per 0.1 mg of BCG, 6×10⁷ CFU per 0.1 mg of BCG, 7×10⁷ CFU per 0.1 mg of BCG, 8×10⁷ CFU per 0.1 mg of BCG, 9×10⁷ CFU per 0.1 mg of BCG, or 1×10⁸ CFU per 0.1 mg of BCG).

In some embodiments, each dose of BCG comprises from about 1×10⁵ CFU per 0.1 mg of BCG to about 1×10⁷ CFU per 0.1 mg of BCG (e.g., about 1×10⁵ CFU per 0.1 mg of BCG, 2×10⁵ CFU per 0.1 mg of BCG, 3×10⁵ CFU per 0.1 mg of BCG, 4×10⁵ CFU per 0.1 mg of BCG, 5×10⁵ CFU per 0.1 mg of BCG, 6×10⁵ CFU per 0.1 mg of BCG, 7×10⁵ CFU per 0.1 mg of BCG, 8×10⁵ CFU per 0.1 mg of BCG, 9×10⁵ CFU per 0.1 mg of BCG, 1×10⁶ CFU per 0.1 mg of BCG, 2×10⁶ CFU per 0.1 mg of BCG, 3×10⁶ CFU per 0.1 mg of BCG, 4×10⁶ CFU per 0.1 mg of BCG, 5×10⁶ CFU per 0.1 mg of BCG, 6×10⁶ CFU per 0.1 mg of BCG, 7×10⁶ CFU per 0.1 mg of BCG, 8×10⁶ CFU per 0.1 mg of BCG, 9×10⁶ CFU per 0.1 mg of BCG, or 1×10⁷ CFU per 0.1 mg of BCG).

In some embodiments, each dose of BCG comprises from about 1×10⁶ CFU per 0.1 mg of BCG to about 9×10⁶ CFU per 0.1 mg of BCG (e.g., about 1×10⁶ CFU per 0.1 mg of BCG, 2×10⁶ CFU per 0.1 mg of BCG, 3×10⁶ CFU per 0.1 mg of BCG, 4×10⁶ CFU per 0.1 mg of BCG, 5×10⁶ CFU per 0.1 mg of BCG, 6×10⁶ CFU per 0.1 mg of BCG, 7×10⁶ CFU per 0.1 mg of BCG, 8×10⁶ CFU per 0.1 mg of BCG, or 9×10⁶ CFU per 0.1 mg of BCG).

In some embodiments, each dose of BCG comprises from about 1.8×10⁶ CFU per 0.1 mg of BCG to about 3.9×10⁶ CFU per 0.1 mg of BCG (e.g., about 1×10⁶ CFU per 0.1 mg of BCG, 1.1×10⁶ CFU per 0.1 mg of BCG, 1.2×10⁶ CFU per 0.1 mg of BCG, 1.3×10⁶ CFU per 0.1 mg of BCG, 1.4×10⁶ CFU per 0.1 mg of BCG, 1.5×10⁶ CFU per 0.1 mg of BCG, 1.6×10⁶ CFU per 0.1 mg of BCG, 1.7×10⁶ CFU per 0.1 mg of BCG, 1.8×10⁶ CFU per 0.1 mg of BCG, 1.9×10⁶ CFU per 0.1 mg of BCG, 2×10⁶ CFU per 0.1 mg of BCG, 2.1×10⁶ CFU per 0.1 mg of BCG, 2.2×10⁶ CFU per 0.1 mg of BCG, 2.3×10⁶ CFU per 0.1 mg of BCG, 2.4×10⁶ CFU per 0.1 mg of BCG, 2.5×10⁶ CFU per 0.1 mg of BCG, 2.6×10⁶ CFU per 0.1 mg of BCG, 2.7×10⁶ CFU per 0.1 mg of BCG, 2.8×10⁶ CFU per 0.1 mg of BCG, 2.9×10⁶ CFU per 0.1 mg of BCG, 3×10⁶ CFU per 0.1 mg of BCG, 3.1×10⁶ CFU per 0.1 mg of BCG, 3.2×10⁶ CFU per 0.1 mg of BCG, 3.3×10⁶ CFU per 0.1 mg of BCG, 3.4×10⁶ CFU per 0.1 mg of BCG, 3.5×10⁶ CFU per 0.1 mg of BCG, 3.6×10⁶ CFU per 0.1 mg of BCG, 3.7×10⁶ CFU per 0.1 mg of BCG, 3.8×10⁶ CFU per 0.1 mg of BCG, or 3.9×10⁶ CFU per 0.1 mg of BCG).

In some embodiments of any of the foregoing aspects or embodiments of the disclosure, the BCG is administered to the subject subcutaneously, intradermally, intravenously, or intraperitoneally.

In some embodiments, prior to administration of the BCG to the subject, the subject:
a) has a chronological age of from about 18 years to about 65 years, or more (e.g., a chronological age of about 18 years, 19 years, 20 years, 21 years, 22 years, 23 years, 24 years, 25 years, 26 years, 27 years, 28 years, 29 years, 30 years, 31 years, 32 years, 33 years, 34 years, 35 years, 36 years, 37 years, 38 years, 39 years, 40 years, 41 years, 42 years, 43 years, 44 years, 45 years, 46 years, 47 years, 48 years, 49 years, 50 years, 51 years, 52 years, 53 years, 54 years, 55 years, 56 years, 57 years, 58 years, 59 years, 60 years, 61 years, 62 years, 63 years, 64 years, 65 years, 66 years, 67 years, 68 years, 69 years, 70 years, or more);
b) has been living with the diabetes for a duration of from about 1 year to about 60 years, or more (e.g., has been living with the diabetes for a duration of from about 5 years to about 60 years, such as for a duration of about 5 years, 6 years, 7 years, 8 years, 9 years, 10 years, 11 years, 12 years, 13 years, 14 years, 15 years, 16 years, 17 years, 18 years, 19 years, 20 years, 21 years, 22 years, 23 years, 24 years, 25 years, 26 years, 27 years, 28 years, 29 years, 30 years, 31 years, 32 years, 33 years, 34 years, 35 years, 36 years, 37 years, 38 years, 39 years, 40 years, 41 years, 42 years, 43 years, 44 years, 45 years, 46 years, 47 years, 48 years, 49 years, 50 years, 51 years, 52 years, 53 years, 54 years, 55 years, 56 years, 57 years, 58 years, 59 years, or 60 years, or more);
c) has tested positive for presence of anti-GAD autoantibodies in peripheral blood;
d) has exhibited a fasting or stimulated c-peptide concentration of about 200 pM or less, such as a fasting or stimulated c-peptide concentration of from about 5 pM to about 200 pM (e.g., a fasting or stimulated c-peptide concentration of about 5 pM, 6 pM, 7 pM, 8 pM, 9 pM, 10 pM, 11 pM, 2 pM, 3 pM, 4 pM, 15 pM, 16 pM, 17 pM, 18 pM, 19 pM, 20 pM, 21 pM, 22 pM, 23 pM, 24 pM, 25 pM, 26 pM, 27 pM, 28 pM, 29 pM, 30 pM, 31 pM, 32 pM, 33 pM, 34 pM, 35 pM, 36 pM, 37 pM, 38 pM, 39 pM, 40 pM, 41 pM, 42 pM, 43 pM, 44 pM, 45 pM, 46 pM, 47 pM, 48 pM, 49 pM, 50 pM, 51 pM, 52 pM, 53 pM, 54 pM, 55 pM, 56 pM, 57 pM, 58 pM, 59 pM, 60 pM, 61 pM, 62 pM, 63 pM, 64 pM, 65 pM, 66 pM, 67 pM, 68 pM, 69 pM, 70 pM, 71 pM, 72 pM, 73 pM, 74 pM, 75 pM, 76 pM, 77 pM, 78 pM, 79 pM, 80 pM, 81 pM, 82 pM, 83 pM, 84 pM, 85 pM, 86 pM, 87 pM, 88 pM, 89 pM, 90 pM, 91 pM, 92 pM, 93 pM, 94 pM, 95 pM, 96 pM, 97 pM, 98 pM, 99 pM, 100 pM, 101 pM, 102 pM, 103 pM, 104 pM, 105 pM, 106 pM, 107 pM, 108 pM, 109 pM, 110 pM, 111 pM, 112 pM, 113 pM, 114 pM, 115 pM, 116 pM, 117 pM, 118 pM, 119 pM, 120 pM, 121 pM, 122 pM, 123 pM, 124 pM, 125 pM, 126 pM, 127 pM, 128 pM, 129 pM, 130 pM, 131 pM, 132 pM, 133 pM, 134 pM, 135 pM, 136 pM, 137 pM, 138 pM, 139 pM, 140 pM, 141 pM, 142 pM, 143 pM, 144 pM, 145 pM, 146 pM, 147 pM, 148 pM, 149 pM, 150 pM, 151 pM, 152 pM, 153 pM, 154 pM, 155 pM, 156 pM, 157 pM, 158 pM, 159 pM, 160 pM, 161 pM, 162 pM, 163 pM, 164 pM, 165 pM, 166 pM, 167 pM, 168 pM, 169 pM, 170 pM, 171 pM, 172 pM, 173 pM, 174 pM, 175 pM, 176 pM, 177 pM, 178 pM, 179 pM, 180 pM, 181 pM, 182 pM, 183 pM, 184 pM, 185 pM, 186 pM, 187 pM, 188 pM, 189 pM, 190 pM, 191 pM, 192 pM, 193 pM, 194 pM, 195 pM, 196 pM, 197 pM, 198 pM, 199 pM, or 200 pM);
e) has exhibited a blood glucose concentration of about 200 mg/dL or greater (e.g., a blood glucose concentration of about 200 mg/dL, 201 mg/dL, 202 mg/dL, 203 mg/dL, 204 mg/dL, 205 mg/dL, 206 mg/dL, 207 mg/dL, 208 mg/dL, 209 mg/dL, 210 mg/dL, 211 mg/dL, 212 mg/dL, 213 mg/dL, 214 mg/dL, 215 mg/dL, 216 mg/dL, 217 mg/dL, 218 mg/dL, 219 mg/dL, 220 mg/dL, 221 mg/dL, 222 mg/dL, 223 mg/dL, 224 mg/dL, 225 mg/dL, 226 mg/dL, 227 mg/dL, 228 mg/dL, 229 mg/dL, 230 mg/dL, 231 mg/dL, 232 mg/dL, 233 mg/dL, 234 mg/dL, 235 mg/dL, 236 mg/dL, 237 mg/dL, 238 mg/dL, 239 mg/dL, 240 mg/dL, 241 mg/dL, 242 mg/dL, 243 mg/dL, 244 mg/dL, 245 mg/dL, 246 mg/dL, 247 mg/dL, 248 mg/dL, 249 mg/dL, 250 mg/dL, or greater);
f) has exhibited an HbA1c level of about 6.5% or greater (e.g., an HbA1c level of 6.5%, 6.6%, 6.7%, 6.8%, 6.9%, 7%, 7.1%, 7.2%, 7.3%, 7.4%, 7.5%, 7.6%, 7.7%, 7.8%, 7.9%, 8%, 8.1%, 8.2%, 8.3%, 8.4%, 8.5%, 8.6%, 8.7%, 8.8%, 8.9%, 9%, 9.1%, 9.2%, 9.3%, 9.4%, 9.5%, 9.6%, 9.7%, 9.8%, 9.9%, 10%, or greater (e.g., an HbA1c level of 47.5 mmol/mol to 86 mmol/mol, or greater)), which identifies the subject as having advanced diabetes (e.g., advanced type 1 diabetes); and/or
g) has exhibited a blood glucose concentration of about 100 mg/dL or greater (e.g., a blood glucose concentration of 100 mg/dL, 101 mg/dL, 102 mg/dL, 103 mg/dL, 104 mg/dL, 105 mg/dL, 106 mg/dL, 107 mg/dL, 108 mg/dL, 109 mg/dL, 110 mg/dL, 111 mg/dL, 112 mg/dL, 113 mg/dL, 114 mg/dL, 115 mg/dL, 116 mg/dL, 117 mg/dL, 118 mg/dL, 119 mg/dL, 120 mg/dL, 121 mg/dL, 122 mg/dL, 123 mg/dL, 124 mg/dL, 125 mg/dL, 126 mg/dL, 127 mg/dL, 128 mg/dL, 129 mg/dL, 130 mg/dL, 131 mg/dL, 132 mg/dL, 133 mg/dL, 134 mg/dL, 135 mg/dL, 136 mg/dL, 137 mg/dL, 138 mg/dL, 139 mg/dL, 140 mg/dL, 141 mg/dL, 142 mg/dL, 143 mg/dL, 144 mg/dL, 145 mg/dL, 146 mg/dL, 147 mg/dL, 148 mg/dL, 149 mg/dL, 150 mg/dL, or greater) in a fasting state, such as a fasting state in which the subject has not eaten for the previous 8-12 hours relative to the time at which the blood glucose concentration is measured.

In some embodiments of any of the foregoing aspects or embodiments of the disclosure, prior to being administered the BCG, the subject is undergoing insulin therapy. For example, prior to being administered the BCG, the subject may be dependent upon the insulin therapy to maintain glucose homeostasis (e.g., the subject may be dependent upon the insulin therapy to maintain a blood glucose concentration of less than 250 mg/dL after eating a meal). In some embodiments, the insulin therapy includes administration of insulin to the subject in one or more doses per day, week, month, or year.

In some embodiments, the insulin is administered to the subject subcutaneously, intravenously, parenterally, or by way of inhalation.

In some embodiments, the insulin is administered to the subject in the form of one or more agents selected from insulin lispro injection (ADMELOG™), insulin glulisine (APIDRA™), insulin aspart (FIASP™), insulin lispro (HUMALOG™), human insulin isophane suspension (HUMULIN N™), insulin glargine (LANTUS™), insulin detemir (LEVEMIR™), insulin degludec (TRESIBA FLEXTOUCH™), 75% insulin lispro protamine suspension/25% insulin lispro injection (HUMALOG MIX 75/25™), 70% human insulin isophane suspension/30% human insulin injection (HUMALOG 70/30™), 50% insulin lispro protamine suspension/50% insulin lispro injection (HUMALOG MIX 50/50™), 70% insulin aspart protamine suspension/30% insulin aspart injection (NOVOLOG MIX 70/30™), 70% insulin degludec/30% insulin aspart (RYZODEG 70/30 FLEXTOUCH™), and 70% NPH human insulin/30% regular human insulin injection (HUMULIN 70/30™).

In some embodiments, following administration of the BCG to the subject, the frequency of the insulin therapy is reduced. For example, prior to administration of the BCG to the subject, the subject may be receiving insulin in one or more doses per day, week, or month, such as in one or more doses per day, from 2 to 10 doses per week, or from 5 to 25 doses per month. Following administration of the BCG to the subject, the frequency of the insulin therapy may be reduced, for example, such that the subject only receives from 1 to 5 doses of insulin per week or from 2 to 10 doses of insulin per month.

In some embodiments, following administration of the BCG to the subject, the quantity of insulin that is administered to the subject per dose is reduced. For example, the subject may be receiving insulin an amount of from about 0.1 to about 0.2 units/kg prior to administration of the BCG to the subject. Following administration of the BCG to the subject, the subject may receive insulin in a reduced quantity per dose, such as an amount of from about 0.01 to about 0.05 units/kg.

In some embodiments, following administration of the BCG to the subject, the insulin therapy is ceased altogether.

In some embodiments of any of the foregoing aspects or embodiments of the disclosure, following administration of the BCG to the subject, the subject exhibits a reduction in HbA1c level, such as a reduction of about 5% or more. For example, in some embodiments, following administration of the BCG to the subject, the subject exhibits a reduction in HbA1c level of from about 5% to about 15%, or more (e.g., a reduction in HbA1c level of about 5%, 6%, 7%, 8%, 9%, 10%, 11%, 12%, 13%, 14%, or 15%).

In some embodiments, the subject exhibits the reduction in HbA1c level at least about 6 months following administration of the BCG to the subject, such as from about 6 months to about 36 months following administration of the BCG to the subject (e.g., about 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, or 36 months, following administration of the BCG to the subject.

In some embodiments, following administration of the BCG to the subject, the subject exhibits a blood glucose concentration of less than 200 mg/dL (e.g., a blood glucose concentration of from about 100 mg/dL to about 200 mg/dL, or less, such as a blood glucose concentration of 100 mg/dL, 101 mg/dL, 102 mg/dL, 103 mg/dL, 104 mg/dL, 105 mg/dL, 106 mg/dL, 107 mg/dL, 108 mg/dL, 109 mg/dL, 110 mg/dL, 111 mg/dL, 112 mg/dL, 113 mg/dL, 114 mg/dL, 115 mg/dL, 116 mg/dL, 117 mg/dL, 118 mg/dL, 119 mg/dL, 120 mg/dL, 121 mg/dL, 122 mg/dL, 123 mg/dL, 124 mg/dL, 125 mg/dL, 126 mg/dL, 127 mg/dL, 128 mg/dL, 129 mg/dL, 130 mg/dL, 131 mg/dL, 132 mg/dL, 133 mg/dL, 134 mg/dL, 135 mg/dL, 136 mg/dL, 137 mg/dL, 138 mg/dL, 139 mg/dL, 140 mg/dL, 141 mg/dL, 142 mg/dL, 143 mg/dL, 144 mg/dL, 145 mg/dL, 146 mg/dL, 147 mg/dL, 148 mg/dL, 149 mg/dL, 150 mg/dL, 151 mg/dL, 152 mg/dL, 153 mg/dL, 154 mg/dL, 155 mg/dL, 156 mg/dL, 157 mg/dL, 158 mg/dL, 159 mg/dL, 160 mg/dL, 161 mg/dL, 162 mg/dL, 163 mg/dL, 164 mg/dL, 165 mg/dL, 166 mg/dL, 167 mg/dL, 168 mg/dL, 169 mg/dL, 170 mg/dL, 171 mg/dL, 172 mg/dL, 173 mg/dL, 174 mg/dL, 175 mg/dL, 176 mg/dL, 177 mg/dL, 178 mg/dL, 179 mg/dL, 180 mg/dL, 181 mg/dL, 182 mg/dL, 183 mg/dL, 184 mg/dL, 185 mg/dL, 186 mg/dL, 187 mg/dL, 188 mg/dL, 189 mg/dL, 190 mg/dL, 191 mg/dL, 192 mg/dL, 193 mg/dL, 194 mg/dL, 195 mg/dL, 196 mg/dL, 197 mg/dL, 198 mg/dL, 199 mg/dL, or 200 mg/dL). In some embodiments, the blood glucose concentration of 200 mg/dL or less is sustained without a need for insulin therapy. In some embodiments, the blood glucose concentration of less than 200 mg/dL is sustained in both a fed and fasting state.

In some embodiments, following administration of the BCG to the subject, the subject exhibits:
a) an increase in glucose uptake (e.g., in a sample of peripheral blood cells obtained from the subject, such as a sample of leukocytes, e.g., monocytes, obtained from the subject), optionally wherein the increase in glucose uptake is from about 5% to about 200%, or more, such as about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 101%, 102%, 103%, 104%, 105%, 106%, 107%, 108%, 109%, 110%, 111%, 112%, 113%, 114%, 115%, 116%, 117%, 118%, 119%, 120%, 121%, 122%, 123%, 124%, 125%, 126%, 127%, 128%, 129%, 130%, 131%, 132%, 133%, 134%, 135%, 136%, 137%, 138%, 139%, 140%, 141%, 142%, 143%, 144%, 145%, 146%, 147%, 148%, 149%, 150%, 151%, 152%, 153%, 154%, 155%, 156%, 157%, 158%, 159%, 160%, 161%, 162%, 163%, 164%, 165%, 166%, 167%, 168%, 169%, 170%, 171%, 172%, 173%, 174%, 175%, 176%, 177%, 178%, 179%, 180%, 181%, 182%, 183%, 184%, 185%, 186%, 187%, 188%, 189%, 190%, 191%, 192%, 193%, 194%, 195%, 196%, 197%, 198%, 199%, or 200%, or more;
b) an reduction in oxidative phosphorylation, presenting, for example, as a reduction in oxygen consumption (e.g., in a sample of peripheral blood cells obtained from the subject, such as a sample of leukocytes, e.g., monocytes, obtained from the subject), optionally wherein the reduction in oxygen consumption is from 5% to about 50%, or more, such as about 5%, 6%, 7%,8%,9%,10%,11%,12%,13%,14%,15%,16%,17%,18%,19%,20%,21%,22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50%, or more; and/or
c) an increase in aerobic glycolysis, presenting, for example, as an increase in extracellular acidification rate (e.g., in a sample of peripheral blood cells obtained from the subject, such as a sample of leukocytes, e.g., monocytes, obtained from the subject), optionally wherein the increase in extracellular acidification rate is from about 5% to about 200%, or more, such as about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 101%, 102%, 103%, 104%, 105%, 106%, 107%, 108%, 109%, 110%, 111%, 112%, 113%, 114%, 115%, 116%, 117%, 118%, 119%, 120%, 121%, 122%, 123%, 124%, 125%, 126%, 127%, 128%, 129%, 130%, 131%, 132%, 133%, 134%, 135%, 136%, 137%, 138%, 139%, 140%, 141%, 142%, 143%, 144%, 145%, 146%, 147%, 148%, 149%, 150%, 151%, 152%, 153%, 154%, 155%, 156%, 157%, 158%, 159%, 160%, 161%, 162%, 163%, 164%, 165%, 166%, 167%, 168%, 169%, 170%, 171%, 172%, 173%, 174%, 175%, 176%, 177%, 178%, 179%, 180%, 181%, 182%, 183%, 184%, 185%, 186%, 187%, 188%, 189%, 190%, 191%, 192%, 193%, 194%, 195%, 196%, 197%, 198%, 199%, or 200%, or more.

In some embodiments of any of the foregoing aspects or embodiments of the disclosure, the diabetes is type 1 diabetes. In some embodiments, the diabetes is type 2 diabetes.

In some embodiments of any of the foregoing aspects or embodiments of the disclosure, the subject has not been previously administered metformin. In some embodiments, the subject is not administered metformin following, or concurrently with, administration of BCG.

In some embodiments of any of the foregoing aspects or embodiments of the disclosure, the subject does not have latent autoimmune diabetes in adults (LADA).

In some embodiments of any of the foregoing aspects or embodiments of the disclosure, the subject does not have or has not previously had bladder cancer.

In a further aspect, the disclosure features a kit containing: (i) one or more unit dosage forms of BCG (e.g., formulated for subcutaneous, intradermal, intravenous, or intraperitoneal injection to the subject) and (ii) a package insert that instructs a user of the kit to administer the BCG to a subject with diabetes in accordance with the method of any of the foregoing aspects or embodiments of the disclosure.

In another aspect, the disclosure features a kit containing: (i) a compound containing glucose covalently bound to a fluorophore and (ii) a package insert that instructs a user of the kit to select a subject with diabetes for treatment with BCG in accordance with a method of the disclosure described above. The fluorophore may contain, for example, a moiety selected from 7-nitrobenz-2-oxa-1,3-diazol-4-yl (NBD), methoxycoumarin, dialkylaminocoumarin, hydroxycoumarin, aminomethylcoumarin acetate (AMCA), 6,8-difluoro-7-hydroxy-3-carboxycoumarin (PACIFIC BLUE™ dye), 5-(dimethylamino)naphthalene-1-sulfonyl (Dansyl), pyrene, 7-amino-3-{[(2,5-dioxopyrrolidin-1-yl)oxy]-2-oxoethyl}-4-methyl-2-oxo-2H-chromene-6-sulfonic acid (ALEXA FLUOR 350™), 6,8-difluoro-7-hydroxy-4-methylcoumarin (MARINA BLUE™ dye), N-(2-aminoethyl)-4-{5-[4-(dimethylamino)phenyl]-1,3-oxazol-2-yl}benzenesulfonamide (DAPOXYL™ dye), 2,3,5,6-Tetramethyl-1H,7H-pyrazolo[1,2-a]pyrazole-1,7-dione (Bimane), 4-{[4-(Diethylamino)phenyl][4-(ethylamino)naphthalen-2-yl]methylidene}-N,N-diethylcyclohexa-2,5-dien-1-iminium (CASCADE BLUE™ dye), tris(N,N-diethylethanaminium) 8-[2-(4-{[(2,5-dioxopyrrolidin-1-yl)oxy]carbonyl}piperidin-1-yl)-2-oxoethoxy]pyrene-1,3,6-trisulfonate (ALEXA FLUOR 405™), N,N-diethylethanaminium [9-{6-[(2,5-dioxopyrrolidin-1-yl)oxy]-6-oxohexyl}-8,8-dimethyl-2-oxo-4-(trifluoromethyl)-8,9-dihydro-2H-benzo[g]chromen-6-yl]methanesulfonate (ALEXA FLUOR 430™), 1-[({4-[(7-nitro-2,1,3-benzoxadiazol-4-yl)amino]phenyl}acetyl)oxy]pyrrolidine-2,5-dione (QSY™ dye), fluorescein, 2-(6-amino-3-iminio-4,5-disulfonato-3H-xanthen-9-yl)-5-((2,3,5,6-tetrafluorophenoxy)carbonyl)benzoate (ALEXA FLUOR 488™), 2′,7′-Difluoro-3′,6′-dihydroxy-3H-spiro[isobenzofuran-1,9′-xanthen]-3-one (OREGON GREEN™ 488), 1,3,5,7,8-pentamethyl-4,4-difluorro-4-bora-3a,4a-diaza-s-indacene (BODIPY™ 493/503), rhodamine green, and rhodamine red. In some embodiments, the compound is 2-[N-(7-nitrobenz-2-oxa-1,3-diazol-4-yl)amino]-2-deoxy-D-glucose. The kit may further contain one or more unit dosage forms of BCG (e.g., formulated for subcutaneous, intradermal, intravenous, or intraperitoneal injection to the subject). In some embodiments, the package insert further instructs a user to administer BCG to the subject identified as likely to respond to the BCG.

In another aspect, the disclosure features a kit containing: (i) one or more agents capable of detecting oxygen consumption in a cell and (ii) a package insert that instructs a user of the kit to select a subject with diabetes for treatment with BCG in accordance with a method of the disclosure described above. The kit may further contain an electron transport chain inhibitor, such as rotenone or antimycin A. The kit may further contain one or more unit dosage forms of BCG (e.g., formulated for a particular route of administration, such as for subcutaneous, intradermal, intravenous, or intraperitoneal injection to the subject). In some embodiments, the package insert further instructs a user to administer BCG to the subject identified as likely to respond to the BCG.

In another aspect, the disclosure features a kit containing: (i) one or more agents capable of detecting extracellular pH in a sample of cells and (ii) a package insert that instructs a user of the kit to select a subject with diabetes for treatment with BCG in accordance with a method of the disclosure described above. The kit may further contain one or more unit dosage forms of BCG (e.g., formulated for subcutaneous, intradermal, intravenous, or intraperitoneal injection to the subject). In some embodiments, the package insert further instructs a user to administer BCG to the subject identified as likely to respond to the BCG.

In some embodiments of any of the foregoing kits, each unit dosage form of BCG contains from about 1×10⁴ colony forming units (CFU) per 0.1 mg of BCG to about 1×10⁸ CFU per 0.1 mg of BCG (e.g., about 1×10⁴ CFU per 0.1 mg of BCG, 2×10⁴ CFU per 0.1 mg of BCG, 3×10⁴ CFU per 0.1 mg of BCG, 4×10⁴ CFU per 0.1 mg of BCG, 5×10⁴ CFU per 0.1 mg of BCG, 6×10⁴ CFU per 0.1 mg of BCG, 7×10⁴ CFU per 0.1 mg of BCG, 8×10⁴ CFU per 0.1 mg of BCG, 9×10⁴ CFU per 0.1 mg of BCG, 1×10⁵ CFU per 0.1 mg of BCG, 2×10⁵ CFU per 0.1 mg of BCG, 3×10⁵ CFU per 0.1 mg of BCG, 4×10⁵ CFU per 0.1 mg of BCG, 5×10⁵ CFU per 0.1 mg of BCG, 6×10⁵ CFU per 0.1 mg of BCG, 7×10⁵ CFU per 0.1 mg of BCG, 8×10⁵ CFU per 0.1 mg of BCG, 9×10⁵ CFU per 0.1 mg of BCG, 1×10⁶ CFU per 0.1 mg of BCG, 2×10⁶ CFU per 0.1 mg of BCG, 3×10⁶ CFU per 0.1 mg of BCG, 4×10⁶ CFU per 0.1 mg of BCG, 5×10⁶ CFU per 0.1 mg of BCG, 6×10⁶ CFU per 0.1 mg of BCG, 7×10⁶ CFU per 0.1 mg of BCG, 8×10⁶ CFU per 0.1 mg of BCG, 9×10⁶ CFU per 0.1 mg of BCG, 1×10⁷ CFU per 0.1 mg of BCG, 2×10⁷ CFU per 0.1 mg of BCG, 3×10⁷ CFU per 0.1 mg of BCG, 4×10⁷ CFU per 0.1 mg of BCG, 5×10⁷ CFU per 0.1 mg of BCG, 6×10⁷ CFU per 0.1 mg of BCG, 7×10⁷ CFU per 0.1 mg of BCG, 8×10⁷ CFU per 0.1 mg of BCG, 9×10⁷ CFU per 0.1 mg of BCG, or 1×10⁸ CFU per 0.1 mg of BCG).

In some embodiments, each unit dosage form of BCG contains from about 1×10⁵ CFU per 0.1 mg of BCG to about 1×10⁷ CFU per 0.1 mg of BCG (e.g., about 1×10⁵ CFU per 0.1 mg of BCG, 2×10⁵ CFU per 0.1 mg of BCG, 3×10⁵ CFU per 0.1 mg of BCG, 4×10⁵ CFU per 0.1 mg of BCG, 5×10⁵ CFU per 0.1 mg of BCG, 6×10⁵ CFU per 0.1 mg of BCG, 7×10⁵ CFU per 0.1 mg of BCG, 8×10⁵ CFU per 0.1 mg of BCG, 9×10⁵ CFU per 0.1 mg of BCG, 1×10⁶ CFU per 0.1 mg of BCG, 2×10⁶ CFU per 0.1 mg of BCG, 3×10⁶ CFU per 0.1 mg of BCG, 4×10⁶ CFU per 0.1 mg of BCG, 5×10⁶ CFU per 0.1 mg of BCG, 6×10⁶ CFU per 0.1 mg of BCG, 7×10⁶ CFU per 0.1 mg of BCG, 8×10⁶ CFU per 0.1 mg of BCG, 9×10⁶ CFU per 0.1 mg of BCG, or 1×10⁷ CFU per 0.1 mg of BCG).

In some embodiments, each unit dosage form of BCG contains from about 1×10⁶ CFU per 0.1 mg of BCG to about 9×10⁶ CFU per 0.1 mg of BCG (e.g., about 1×10⁶ CFU per 0.1 mg of BCG, 2×10⁶ CFU per 0.1 mg of BCG, 3×10⁶ CFU per 0.1 mg of BCG, 4×10⁶ CFU per 0.1 mg of BCG, 5×10⁶ CFU per 0.1 mg of BCG, 6×10⁶ CFU per 0.1 mg of BCG, 7×10⁶ CFU per 0.1 mg of BCG, 8×10⁶ CFU per 0.1 mg of BCG, or 9×10⁶ CFU per 0.1 mg of BCG).

In some embodiments, each unit dosage form of BCG contains from about 1.8×10⁶ CFU per 0.1 mg of BCG to about 3.9×10⁶ CFU per 0.1 mg of BCG (e.g., about 1×10⁶ CFU per 0.1 mg of BCG, 1.1×106 CFU per 0.1 mg of BCG, 1.2×10⁶ CFU per 0.1 mg of BCG, 1.3×10⁶ CFU per 0.1 mg of BCG, 1.4×106 CFU per 0.1 mg of BCG, 1.5×10⁶ CFU per 0.1 mg of BCG, 1.6×10⁶ CFU per 0.1 mg of BCG, 1.7×106 CFU per 0.1 mg of BCG, 1.8×10⁶ CFU per 0.1 mg of BCG, 1.9×10⁶ CFU per 0.1 mg of BCG, 2×10⁶ CFU per 0.1 mg of BCG, 2.1×10⁶ CFU per 0.1 mg of BCG, 2.2×10⁶ CFU per 0.1 mg of BCG, 2.3×10⁶ CFU per 0.1 mg of BCG, 2.4×10⁶ CFU per 0.1 mg of BCG, 2.5×10⁶ CFU per 0.1 mg of BCG, 2.6×10⁶ CFU per 0.1 mg of BCG, 2.7×10⁶ CFU per 0.1 mg of BCG, 2.8×10⁶ CFU per 0.1 mg of BCG, 2.9×10⁶ CFU per 0.1 mg of BCG, 3×10⁶ CFU per 0.1 mg of BCG, 3.1×10⁶ CFU per 0.1 mg of BCG, 3.2×10⁶ CFU per 0.1 mg of BCG, 3.3×10⁶ CFU per 0.1 mg of BCG, 3.4×10⁶ CFU per 0.1 mg of BCG, 3.5×10⁶ CFU per 0.1 mg of BCG, 3.6×10⁶ CFU per 0.1 mg of BCG, 3.7×10⁶ CFU per 0.1 mg of BCG, 3.8×10⁶ CFU per 0.1 mg of BCG, or 3.9×10⁶ CFU per 0.1 mg of BCG).

### Definitions

As used herein, the term “about” refers to a value that is no more than 10% above or below the value being described. For example, the term “about 5 mg” indicates a range of from 4.5 mg to 5.5 mg.

As used herein, the term “aerobic glycolysis” refers to a metabolic process in which a cell produces energy (e.g., in the form of high-energy phosphodiester bonds, such as those in adenosine trisphosphate or a molecule of similar structure) by way of glycolysis, even though the cell has access to ambient oxygen. Empirically, aerobic glycolysis manifests as an increase in extracellular acidification, resulting, for example, from an accumulation of pyruvic acid and, upon fermentation, lactic acid. Exemplary assays that can be used to monitor aerobic glycolysis are described in TeSlaa and Teitell, Methods in Enzymology 542:91-114 (2014), the disclosure of which is incorporated herein by reference, as well as in Example 1, below.

As used herein, the term “age of onset” refers to the age of a subject (e.g., a human subject) when the subject first develops a particular disease or pathological condition (e.g., diabetes, such as type 1 diabetes), which may be detected by the presence of one or more symptoms of the disease or pathological condition. The subject's age of onset of a particular disease or pathological condition need not be the same as the age of the subject when symptoms first manifest. For example, in some embodiments, a subject may develop a disease or pathological condition at certain point in time, and may not show symptoms of the disease or condition until later in life (e.g., one or more days, weeks, months, or years later in life).

As used herein, the term “BCG” refers to Bacillus Calmette-Guerin, which is a preparation of Mycobacterium bovis, an attenuated strain of Mycobacterium turberculosis, that is not virulent in humans, but is rather, e.g., a vaccine. Examples of BCG include a variety of substrains that have been developed by genetic manipulation, including, e.g., the Pasteur, Phipps, Frappier, Mexico, Birkhaug, Sweden, Moreau, Japan-Tokyo, Copenhagen, TICE, Sanofi, Aventis, Connaught, RIVM, Russian, Evans, MMC, Moreau, and Glaxo substrains of BCG, among others, as well as genetic variants of these substrains. “BCG” also refers to variants of This invention also includes Mycobacterium turberculosis itself that has been genetically modified to no longer be virulent. BCG substrains, as well as the genetic differences between these substrains, are known in the art and are described, e.g., in Castillo-Rodal, et al., Infect Immun. 74(3):1718-1724 (2006); as well as in Zhang, et al., Tubercle and Lung Disease 76(1):43-50 (1995); the disclosures of each of which are incorporated herein by reference.

As used herein, the term “chronological age” refers to the current age of a subject (e.g., a human subject, such as a human subject with diabetes (e.g., type 1 diabetes)). A subject's current or chronological age may be distinct from the age of the subject at the onset of a particular disease or pathological condition (e.g., diabetes, such as type 1 diabetes).

As used herein, the term “colony forming unit” (CFU) refers to at least one cell that is capable of giving rise to a population of genetically identical cells by mitotic cell proliferation. Colony forming units include a single cell, but may also be an aggregation of cells, such as a colony.

As used herein, the terms “conservative mutation,” “conservative substitution,” or “conservative amino acid substitution” refer to a substitution of one or more amino acids for one or more different amino acids that exhibit similar physicochemical properties, such as polarity, electrostatic charge, and/or steric volume. These properties are summarized for each of the twenty naturally-occurring amino acids in Table 1 below.

TABLE 1
Representative physicochemical properties
of naturally-occurring amino acids

| | | | | Electrostatic | |
|---|---|---|---|---|---|
| | 3 | 1 | Side- | character at | |
| | Letter | Letter | chain | physiological | Steric |
| Amino Acid | Code | Code | Polarity | pH (7.4) | Volume^{†} |
| Alanine | Ala | A | nonpolar | neutral | small |
| Arginine | Arg | R | polar | cationic | large |
| Asparagine | Asn | N | polar | neutral | intermediate |
| Aspartic acid | Asp | D | polar | anionic | intermediate |
| Cysteine | Cys | C | nonpolar | neutral | intermediate |
| Glutamic acid | Glu | E | polar | anionic | intermediate |
| Glutamine | Gln | Q | polar | neutral | intermediate |
| Glycine | Gly | G | nonpolar | neutral | small |
| Histidine | His | H | polar | Both neutral | large |
| | | | | and cationic | |
| | | | | forms in | |
| | | | | equilibrium | |
| | | | | at pH 7.4 | |
| Isoleucine | Ile | I | nonpolar | neutral | large |
| Leucine | Leu | L | nonpolar | neutral | large |
| Lysine | Lys | K | polar | cationic | large |
| Methionine | Met | M | nonpolar | neutral | large |
| Phenylalanine | Phe | F | nonpolar | neutral | large |
| Proline | Pro | P | nonpolar | neutral | intermediate |
| Serine | Ser | S | polar | neutral | small |
| Threonine | Thr | T | polar | neutral | intermediate |
| Tryptophan | Trp | W | nonpolar | neutral | bulky |
| Tyrosine | Tyr | Y | polar | neutral | large |
| Valine | Val | V | nonpolar | neutral | intermediate |

^{†}based on volume in A³: 50-100 is small, 100-150 is intermediate, 150-200 is large, and >200 is bulky

From this table it is appreciated that the conservative amino acid families include, e.g., (i) G, A, V, L, I, P, and M; (ii) D and E; (iii) C, S and T; (iv) H, K and R; (v) N and Q; and (vi) F, Y and W. A conservative mutation or substitution is therefore one that substitutes one amino acid for a member of the same amino acid family (e.g., a substitution of Ser for Thr or Lys for Arg).

As used herein, the term “conjugate” refers to a compound formed by the chemical bonding of a reactive functional group of one molecule with an appropriately reactive functional group of another molecule. Conjugates may additionally be produced, e.g., as two polypeptide domains covalently bound to one another as part of a single polypeptide chain that is synthesized by the translation of a single RNA transcript encoding both polypeptides in frame with one another.

As used herein, the terms “diagnose,” “diagnosis”, and the like refer to a determination that a subject (e.g., a human subject) has a particular disease or pathological condition (e.g., diabetes, such as type 1 diabetes). A diagnosis may be made, for example, by way of a physician observing a subject suspected of having a disease or pathological condition, and may be confirmed by a finding that the subject exhibits one or more symptoms of the disease or condition. For example, in the context of a subject (e.g., a human subject) suspected of having diabetes (e.g., type 1 diabetes), a subject may be diagnosed as having this disease on the basis of a determination that the subject exhibits one or more, or all, of the following symptoms: (i) a blood glucose concentration of 200 mg/dL or more, such as a blood glucose concentration of 250 mg/dL or more; (ii) a glycated hemoglobin (HbA1c) level of 6.5% or greater, such as an HbA1c level of from about 6.5% to about 15%, or more; (iii) a blood glucose concentration of about 100 mg/dL or greater when the subject is in a fasting state; and/or (iv) a presence of anti-glutamic acid decarboxylase (GAD) autoantibodies in peripheral blood.

As used herein, the term “dose” refers to the quantity of a therapeutic agent, such as BCG, that is administered to a subject at a particular instant for the treatment of a disorder or condition, such as diabetes (e.g., type 1 diabetes). A therapeutic agent as described herein (e.g., BCG) may be administered in a single dose or in multiple doses over the course of a treatment period. In each case, the therapeutic agent may be administered using one or more “unit dosage forms” of the therapeutic agent, a term that refers to a one or more discrete compositions containing a therapeutic agent that collectively constitute a single dose of the agent. For instance, a single dose of 1×10⁶ colony forming units (CFU) of BCG may be administered using, e.g., two unit dosage forms each containing 5×10⁵ CFU of BCG, four unit dosage forms each containing 2.5×10⁵ CFU of BCG, 10 unit dosage forms each containing 1×10⁵ CFU of BCG, and the like. The unit dosage forms may be, for example, liquid dosage forms, such as suspensions formulated for subcutaneous, intradermal, intravenous, or intraperitoneal injection, among other routes of administration described herein.

As used herein, the term “endogenous” describes a molecule (e.g., a polypeptide, nucleic acid, or cofactor) that is found naturally in a particular organism (e.g., a human) or in a particular location within an organism (e.g., an organ, a tissue, or a cell, such as a human cell).

As used herein, the term “exogenous” describes a molecule (e.g., a polypeptide, nucleic acid, or cofactor) that is not found naturally in a particular organism (e.g., a human) or in a particular location within an organism (e.g., an organ, a tissue, or a cell, such as a human cell). Exogenous materials include those that are provided from an external source to an organism or to cultured matter extracted therefrom.

As used herein, the term “glucose uptake” refers to the internalization of glucose by a cell, for example, as facilitated by glucose transport proteins, such as the GLUT1 transporter. Empirically, glucose uptake by a cell can be monitored using a variety of methods. For example, glucose uptake may be visualized by measuring the fluorescence of a cell after incubating the cell in the presence of a compound containing glucose covalently bound to a fluorophore, such as a fluorophore known herein. Examples of such compounds include 2-[N-(7-nitrobenz-2-oxa-1,3-diazol-4-yl)amino]-2-deoxy-D-glucose (2-NBDG). Exemplary fluorescence assays that can be used to monitor glucose uptake by a cell are described in Zou et al., Journal of Biochemical and Biophysical Methods 64:207-215 (2005), the disclosure of which is incorporated by reference herein, as well as in Example 1, below.

As used herein, the term “insulin therapy” refers to administration of insulin to a subject (e.g., a human subject) with diabetes (e.g., type 1 diabetes), for example, to maintain glucose homeostasis in the subject. The insulin may be administered to the subject periodically over the course of a treatment period, such as in one or more doses per day, week, month, or year. The insulin may be administered to the subject by way of subcutaneous, intradermal, intravenous, or intraperitoneal injection, by way of inhalation, or by way of another route of administration described herein.

As used herein, the term “juvenile onset diabetes” refers to diabetes (e.g., type 1 diabetes) that is developed by a subject (e.g., a human subject) during, or prior to the onset of, puberty. Puberty may manifest empirically by a showing of one or more secondary sex characteristics, such as those described, for example, in Daniel et al., Journal of Pediatrics 96:1074-1078 (1980), and Harlan et al., Journal of Pediatrics 95:287-289 (1979), the disclosures of each of which are incorporated herein by reference as they pertain to attributes that identify an individual as pubescent.

As used herein, the term “latent autoimmune diabetes in adults” (LADA, also known as “late autoimmune disease of adults” and “type 1.5 diabetes”) refers to diabetes (e.g., type 1 diabetes) that is developed by a subject (e.g., a human subject) in adults (e.g., subjects aged 21 years or more) with a mean age of onset of 30 years. Subjects with LADA have a slower decline in pancreas function and may become insulin dependent.

As used herein, the term “oxidative phosphorylation” refers to the use of oxygen by a cell for the production of energy, for example, in the form of high-energy phosphodiester bonds, such as those present in adenosine trisphosphate or a molecule of similar structure. Oxidative phosphorylation may include the use of oxygen as a final acceptor of electrons in flux through the electron transport chain. Empirically, oxidative phosphorylation may be observed in a sample of cells by monitoring the consumption of oxygen by the cells. Exemplary assays that are used for monitoring the rate of oxygen consumption in a cell are described, for example, in Ehrismann et al., Biochemical Journal 401:227-234 (2007), the disclosure of which is incorporated herein by reference in its entirety, as well as in Example 1, below.

As used herein, the term “subject” refers to an organism that receives treatment (e.g., by administration of a therapeutic agent described herein, such as BCG) for a particular disease or condition, such as diabetes (e.g., type 1 diabetes). Examples of subjects that may be treated using the compositions and methods of the disclosure include mammals, such as humans, primates, pigs, goats, rabbits, hamsters, cats, dogs, guinea pigs, members of the bovidae family (such as cattle, bison, buffalo, and yaks, among others), cows, sheep, horses, and bison, among others. In the context of a subject that is receiving treatment for diabetes (e.g., type 1 diabetes), the subject may exhibit one or more, or all, of the following attributes prior to the onset or completion of a therapeutic regimen described herein: (i) a blood glucose concentration of 200 mg/dL or more, such as a blood glucose concentration of 250 mg/dL or more; (ii) a glycated hemoglobin (HbA1c) level of 6.5% or greater, such as an HbA1c level of from about 6.5% to about 15%, or more; (iii) a blood glucose concentration of about 100 mg/dL or greater when the subject is in a fasting state; and/or (iv) a presence of anti-GAD autoantibodies in peripheral blood.

As used herein, the term “percent (%) sequence identity” refers to the percentage of amino acid (or nucleic acid) residues of a candidate sequence that are identical to the amino acid (or nucleic acid) residues of a reference sequence after aligning the sequences and introducing gaps, if necessary, to achieve the maximum percent sequence identity (e.g., gaps can be introduced in one or both of the candidate and reference sequences for optimal alignment and non-homologous sequences can be disregarded for comparison purposes). Alignment for purposes of determining percent sequence identity can be achieved in various ways that are within the skill in the art, for instance, using publicly available computer software, such as BLAST, ALIGN, or Megalign (DNASTAR) software. Those skilled in the art can determine appropriate parameters for measuring alignment, including any algorithms needed to achieve maximal alignment over the full length of the sequences being compared. For example, a reference sequence aligned for comparison with a candidate sequence may show that the candidate sequence exhibits from 50% to 100% sequence identity across the full length of the candidate sequence or a selected portion of contiguous amino acid (or nucleic acid) residues of the candidate sequence. The length of the candidate sequence aligned for comparison purposes may be, for example, at least 30%, (e.g., 30%, 40, 50%, 60%, 70%, 80%, 90%, or 100%) of the length of the reference sequence. When a position in the candidate sequence is occupied by the same amino acid residue as the corresponding position in the reference sequence, then the molecules are identical at that position.

As used herein in the context of administration of a therapeutic agent, the term “periodically” refers to administration of the agent two or more times over the course of a treatment period (e.g., two or more times daily, weekly, monthly, or yearly).

As used herein, the term “pharmaceutical composition” refers to a mixture containing a therapeutic agent (e.g., BCG) to be administered to a subject, such as a mammal, e.g., a human, in order to prevent, treat or control a particular disease or condition affecting the mammal, such as diabetes (e.g., type 1 diabetes).

As used herein, the term “pharmaceutically acceptable” refers to those compounds, materials, compositions, and/or dosage forms that are suitable for contact with the tissues of a subject, such as a mammal (e.g., a human) without excessive toxicity, irritation, allergic response, and other complications commensurate with a reasonable benefit/risk ratio.

As used herein, the term “pharmacokinetic profile” refers to the absorption, distribution, metabolism, and clearance of a therapeutic agent (e.g., BCG) over time following administration of the therapeutic agent to a subject.

As used herein, a “reference” quantity, such as a “reference rate of glucose uptake,” a “reference rate of oxidative phosphorylation,” and a “reference rate of aerobic glycolysis,” refers to a measurement of a quantity in a healthy subject (e.g., a subject that does not have type 1 diabetes, such as a subject that does not have any form of diabetes), such as a rate of one or more metabolic processes in the subject, that can be compared with a measurement of the same quantity in a subject being considered for treatment with a therapeutic agent (e.g., BCG) in order to assess the likelihood that the subject will respond to the therapeutic agent. For example, using the compositions and methods disclosed herein, a subject with diabetes (e.g., type 1 diabetes) may be assessed for their likelihood of responding to treatment with BCG by comparing the rate of glucose uptake, oxidative phosphorylation, and/or aerobic glycolysis exhibited by the subject to a reference rate of glucose uptake, a reference rate of oxidative phosphorylation, and/or a reference rate of aerobic glycolysis, respectively, in order to determine the likelihood that the subject will benefit from treatment with the BCG. In this context, a finding that the subject exhibits reduced glucose uptake relative to a reference rate of glucose uptake, heightened oxidative phosphorylation relative to a reference rate of oxidative phosphorylation, and/or reduced aerobic glycolysis relative to a reference rate of aerobic glycolysis identifies the subject as one that is likely to benefit from treatment with the BCG. A healthy subject, such as a subject that does not have diabetes, and from whom a reference sample can be obtained, is referred to herein as a “reference subject.”

As used herein, the terms “respond,” “benefit,” and the like are used interchangeably in the context of a subject, such as a human subject undergoing therapy for the treatment of diabetes (e.g., type 1 diabetes). These terms refer to clinical improvement (as assessed using an art-recognized metric, such as one or more of the metrics described herein) in the subject's condition resulting from administration of a therapeutic agent, such as BCG. Exemplary benefits in the context of a subject undergoing treatment for diabetes (e.g., type 1 diabetes) by way of administration of BCG include, without limitation, (i) an increase in glucose uptake relative to a pre-treatment level of glucose uptake (e.g., in a sample of peripheral blood cells obtained from the subject, such as a sample of leukocytes, e.g., monocytes, obtained from the subject), such as an increase in glucose uptake relative to a pre-treatment level of glucose uptake of from about 5% to about 200%, or more (e.g., about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 101%, 102%, 103%, 104%, 105%, 106%, 107%, 108%, 109%, 110%, 111%, 112%, 113%, 114%, 115%, 116%, 117%, 118%, 119%, 120%, 121%, 122%, 123%, 124%, 125%, 126%, 127%, 128%, 129%, 130%, 131%, 132%, 133%, 134%, 135%, 136%, 137%, 138%, 139%, 140%, 141%, 142%, 143%, 144%, 145%, 146%, 147%, 148%, 149%, 150%, 151%, 152%, 153%, 154%, 155%, 156%, 157%, 158%, 159%, 160%, 161%, 162%, 163%, 164%, 165%, 166%, 167%, 168%, 169%, 170%, 171%, 172%, 173%, 174%, 175%, 176%, 177%, 178%, 179%, 180%, 181%, 182%, 183%, 184%, 185%, 186%, 187%, 188%, 189%, 190%, 191%, 192%, 193%, 194%, 195%, 196%, 197%, 198%, 199%, or 200%, or more); (ii) a reduction in oxidative phosphorylation relative to a pre-treatment level of oxidative phosphorylation, presenting, for example, as a reduction in oxygen consumption relative to a pre-treatment level of oxygen consumption (e.g., in a sample of peripheral blood cells obtained from the subject, such as a sample of leukocytes, e.g., monocytes, obtained from the subject), such as a reduction in oxygen consumption relative to a pre-treatment level of oxygen consumption of from about 5% to about 50%, or more (e.g., about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50%, or more); and/or (iii) an increase in aerobic glycolysis relative to a pre-treatment level of aerobic glycolysis presenting, for example, as an increase in extracellular acidification rate relative to a pre-treatment level of extracellular acidification rate (e.g., in a sample of peripheral blood cells obtained from the subject, such as a sample of leukocytes, e.g., monocytes, obtained from the subject), such as an increase in extracellular acidification rate relative to a pre-treatment level of extracellular acidification rate of from about 5% to about 200%, or more (e.g., about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 101%, 102%, 103%, 104%, 105%, 106%, 107%, 108%, 109%, 110%, 111%, 112%, 113%, 114%, 115%, 116%, 117%, 118%, 119%, 120%, 121%, 122%, 123%, 124%, 125%, 126%, 127%, 128%, 129%, 130%, 131%, 132%, 133%, 134%, 135%, 136%, 137%, 138%, 139%, 140%, 141%, 142%, 143%, 144%, 145%, 146%, 147%, 148%, 149%, 150%, 151%, 152%, 153%, 154%, 155%, 156%, 157%, 158%, 159%, 160%, 161%, 162%, 163%, 164%, 165%, 166%, 167%, 168%, 169%, 170%, 171%, 172%, 173%, 174%, 175%, 176%, 177%, 178%, 179%, 180%, 181%, 182%, 183%, 184%, 185%, 186%, 187%, 188%, 189%, 190%, 191%, 192%, 193%, 194%, 195%, 196%, 197%, 198%, 199%, or 200%, or more).

As used herein, the terms “treat,” “treatment,” and the like refer to therapeutic treatment, in which the object is to inhibit or slow down (lessen) an undesired physiological change or disorder, such as diabetes (e.g., type 1 diabetes). Beneficial or desired clinical results of treatment include, without limitation, alleviation of symptoms, diminishment of extent of disease, stabilized (e.g., not worsening) state of disease, delay or slowing of disease progression, amelioration or palliation of the disease state, and remission (whether partial or total), whether detectable or undetectable. For example, in the context of a subject that is treated for diabetes (e.g., type 1 diabetes), clinical indicators of successful treatment include a determination that, following administration of a therapeutic agent described herein (e.g., BCG), the subject exhibits one or more of: (i) an increase in glucose uptake relative to a pre-treatment level of glucose uptake (e.g., in a sample of peripheral blood cells obtained from the subject, such as a sample of leukocytes, e.g., monocytes, obtained from the subject), such as an increase in glucose uptake relative to a pre-treatment level of glucose uptake of from about 5% to about 200%, or more (e.g., about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 101%, 102%, 103%, 104%, 105%, 106%, 107%, 108%, 109%, 110%, 111%, 112%, 113%, 114%, 115%, 116%, 117%, 118%, 119%, 120%, 121%, 122%, 123%, 124%, 125%, 126%, 127%, 128%, 129%, 130%, 131%, 132%, 133%, 134%, 135%, 136%, 137%, 138%, 139%, 140%, 141%, 142%, 143%, 144%, 145%, 146%, 147%, 148%, 149%, 150%, 151%, 152%, 153%, 154%, 155%, 156%, 157%, 158%, 159%, 160%, 161%, 162%, 163%, 164%, 165%, 166%, 167%, 168%, 169%, 170%, 171%, 172%, 173%, 174%, 175%, 176%, 177%, 178%, 179%, 180%, 181%, 182%, 183%, 184%, 185%, 186%, 187%, 188%, 189%, 190%, 191%, 192%, 193%, 194%, 195%, 196%, 197%, 198%, 199%, or 200%, or more); (ii) a reduction in oxidative phosphorylation relative to a pre-treatment level of oxidative phosphorylation, presenting, for example, as a reduction in oxygen consumption relative to a pre-treatment level of oxygen consumption (e.g., in a sample of peripheral blood cells obtained from the subject, such as a sample of leukocytes, e.g., monocytes, obtained from the subject), such as a reduction in oxygen consumption relative to a pre-treatment level of oxygen consumption of from about 5% to about 50%, or more (e.g., about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50%, or more); and (iii) an increase in aerobic glycolysis relative to a pre-treatment level of aerobic glycolysis, presenting, for example, as an increase in extracellular acidification rate relative to a pre-treatment level of extracellular acidification rate (e.g., in a sample of peripheral blood cells obtained from the subject, such as a sample of leukocytes, e.g., monocytes, obtained from the subject), such as an increase in extracellular acidification rate relative to a pre-treatment level of extracellular acidification rate of from about 5% to about 200%, or more (e.g., about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 101%, 102%, 103%, 104%, 105%, 106%, 107%, 108%, 109%, 110%, 111%, 112%, 113%, 114%, 115%, 116%, 117%, 118%, 119%, 120%, 121%, 122%, 123%, 124%, 125%, 126%, 127%, 128%, 129%, 130%, 131%, 132%, 133%, 134%, 135%, 136%, 137%, 138%, 139%, 140%, 141%, 142%, 143%, 144%, 145%, 146%, 147%, 148%, 149%, 150%, 151%, 152%, 153%, 154%, 155%, 156%, 157%, 158%, 159%, 160%, 161%, 162%, 163%, 164%, 165%, 166%, 167%, 168%, 169%, 170%, 171%, 172%, 173%, 174%, 175%, 176%, 177%, 178%, 179%, 180%, 181%, 182%, 183%, 184%, 185%, 186%, 187%, 188%, 189%, 190%, 191%, 192%, 193%, 194%, 195%, 196%, 197%, 198%, 199%, or 200%, or more).

As used herein, the term “treatment period” refers to a duration of time over which a subject may be periodically administered a therapeutic agent (e.g., BCG). Treatment periods as described herein may have a duration of, for example, several days, weeks, months, or years. For instance, a treatment period for administration of BCG to a subject (e.g., a mammal, such as a human) having diabetes (e.g., type 1 diabetes) may have a duration of one or more years, such as a duration of up to five years. In some embodiments, the subject is administered a plurality of doses of BCG over the course of the multi-year treatment period. The doses may be evenly spaced apart or unevenly spaced apart.

For example, in some embodiments, BCG is administered to the subject in at least first, second, and third doses, in which (i) the second dose of the BCG is administered to the subject from about 1 day to about 1 year after administration of the first dose of the BCG to the subject (e.g., from about 1 week to about 12 weeks after administration of the first dose of the BCG to the subject, from about 2 weeks to about 8 weeks after administration of the first dose of the BCG to the subject, from about 3 weeks to about 6 weeks after administration of the first dose of the BCG to the subject, or about 4 weeks after administration of the first dose of the BCG to the subject) and (ii) the third dose of the BCG is administered to the subject from about 2 months to about 72 months after administration of the second dose of the BCG to the subject (e.g., from about 3 months to about 48 months after administration of the second dose of the BCG to the subject, from about 6 months to about 36 months after administration of the second dose of the BCG to the subject, from about 8 months to about 16 months after administration of the second dose of the BCG to the subject, or about 12 months after administration of the second dose of the BCG to the subject. The BCG may further be administered to the subject in at least fourth, fifth, and sixth doses, in which (iii) the fourth dose of the BCG is administered to the subject from about 2 months to about 72 months after administration of the third dose of the BCG to the subject (e.g., from about 3 months to about 48 months after administration of the third dose of the BCG to the subject, from about 6 months to about 36 months after administration of the third dose of the BCG to the subject, from about 8 months to about 16 months after administration of the third dose of the BCG to the subject, or about 12 months after administration of the third dose of the BCG to the subject), (iv) the fifth dose of the BCG is administered to the subject from about 2 months to about 72 months after administration of the fourth dose of the BCG to the subject (e.g., from about 3 months to about 48 months after administration of the fourth dose of the BCG to the subject, from about 6 months to about 36 months after administration of the fourth dose of the BCG to the subject, from about 8 months to about 16 months after administration of the fourth dose of the BCG to the subject, or about 12 months after administration of the fourth dose of the BCG to the subject), and (v) the sixth dose of the BCG is administered to the subject from about 2 months to about 72 months after administration of the fifth dose of the BCG to the subject (e.g., from about 3 months to about 48 months after administration of the fifth dose of the BCG to the subject, from about 6 months to about 36 months after administration of the fifth dose of the BCG to the subject, from about 8 months to about 16 months after administration of the fifth dose of the BCG to the subject, or about 12 months after administration of the fifth dose of the BCG to the subject).

As used herein, the term “vector” includes a nucleic acid vector, e.g., a DNA vector, such as a plasmid, a RNA vector, virus or other suitable replicon (e.g., viral vector). A variety of vectors have been developed for the delivery of polynucleotides encoding exogenous proteins into a prokaryotic or eukaryotic cell. Examples of such expression vectors are disclosed in, e.g., WO 1994/11026; incorporated herein by reference. Expression vectors described herein contain a polynucleotide sequence as well as, e.g., additional sequence elements used for the expression of proteins and/or the integration of these polynucleotide sequences into the genome of a mammalian cell. Certain vectors that can be used for the expression of antibodies and antibody fragments described herein include plasmids that contain regulatory sequences, such as promoter and enhancer regions, which direct gene transcription. Other useful vectors for expression of antibodies and antibody fragments contain polynucleotide sequences that enhance the rate of translation of these genes or improve the stability or nuclear export of the mRNA that results from gene transcription. These sequence elements include, e.g., 5′ and 3′ untranslated regions, an internal ribosomal entry site (IRES), and polyadenylation signal site in order to direct efficient transcription of the gene carried on the expression vector. The expression vectors described herein may also contain a polynucleotide encoding a marker for selection of cells that contain such a vector. Examples of a suitable marker include genes that encode resistance to antibiotics, such as ampicillin, chloramphenicol, kanamycin, or nourseothricin.

### BRIEF DESCRIPTION OF THE FIGURES

FIG. 1A is a timeline showing a schedule of in vivo BCG treatments (3 vaccinations) and corresponding five serial blood samples obtained during a 56 week-long monitoring study of type 1 diabetic (T1D) human subjects (see Example 1, below). The subjects were treated with BCG at week 0, at week 4, and at 1 year. Blood samples were collected at serial visit times (V1-V5): V1 (0 weeks), V2 (10 weeks), V3 (26 weeks), V4 (52 weeks), V5 (56 weeks). The total number of in vivo collected clinical trial blood samples (n) is 15.

FIG. 1B shows a set of graphs illustrating in vitro glucose uptake by untreated monocytes obtained from T1D subjects and non-diabetic controls (NDC) at baseline (left) compared to following treatment with BCG (right). Untreated monocytes from T1D subjects have poorer glucose utilization than NDC, as measured by uptake via the fluorescent glucose derivative 2-NBDG. After overnight culture, BCG increased 2-NBDG uptake in monocytes of both T1 Ds (n=21) and NDCs (n=6) unpaired, 2-tailed T test, P=0.02 (left), P=0.96 (right).

FIG. 1C shows a set of graphs illustrating in vitro glucose uptake in monocytes of non-diabetic controls (NDC) (left) and T1D subjects (right) with and without BCG exposure. Monocytes were isolated from T1D and NDC subjects, cultured overnight with or without BCG, and analyzed for glucose uptake via the fluorescent glucose derivative 2-NBDG. BCG increased 2-NBDG uptake in monocytes of both T1D (n=21) and NDC (n=6); unpaired, 2-tailed t-test, P=0.04 (left), P<0.0001 (right)).

FIG. 1D is a graph showing a longitudinal in vivo analysis of glucose uptake by isolated monocytes harvested from a single T1D subject receiving serial BCG vaccinations (samples at V1,2,3,6). For this analysis, additional samples were collected at week 78 (V6). In vitro glucose uptake was quantified using 2-NBDG uptake assays and showed a gradual increase over time.

FIG. 1E is a set of graphs showing the results of representative oxygen consumption rate (OCR) and extracellular acidification rate (ECAR) assays using T1D monocytes and the SEAHORSE™ XFp analyzer. Isolated monocytes from a T1D subject were cultured for 24 hours with or without BCG, after which the OCR and ECAR were determined. At the arrow, Rotenone and AntiMycin A (Rot/AA) were added. These reagents inhibit the electron transfer chain and, as a consequence, reduce oxidative phosphorylation, and thus, oxygen consumption. BCG treatment reduces the overall oxygen consumption, indicating a switch from oxidative phosphorylation to aerobic glycolysis.

FIG. 1F is a graph showing baseline OCR, a marker of oxidative phosphorylation, in monocytes of untreated T1D subjects as compared to NDC cells. The graph shows basal OCR results. Average OCR is abnormally high in T1D cells (n=61) vs NDC cells (n=16); unpaired t-test, 2-tailed P=0.002).

FIG. 1G is a graph demonstrating that BCG increases ECAR in cultured primary monocytes from T1D subjects and control subjects. T1D or control monocytes were cultured with and without BCG for 7 days and analyzed using a SEAHORSE™ XFp analyzer. The increase in acidification indicates increased aerobic glycolysis after BCG treatment (total number of T1D subjects n=42; paired t-test, P=0.0004).

Level of significance in figures is indicated by asterisk(s): * p<0.05; ** p<0.01; *** p<0.001; **** p<0.0001.

FIG. 2A is a series of graphs showing the effect of age of onset on type 1 diabetic subject response to BCG treatment. While age of onset (AOO) stratifies subjects that are likely to respond from those that are less likely to respond, chronological age and duration of diabetic condition have no effect on BCG response rate. Values along the y-axis of each graph represent HbA1c concentration. The graphs compare changes in HbA1c among type 1 diabetic subjects treated with BCG in phase 1 and phase 2 clinical trials as compared to untreated controls.

FIG. 2B is a graph comparing the glucose uptake of untreated monocytes (left) and BCG-treated monocytes (right) in a sample obtained from a type 1 diabetic subject. Glucose uptake was monitored using a fluorescent glucose conjugate internalization assay.

FIG. 2C is a graph comparing the glucose uptake of untreated monocytes (left) and BCG-treated monocytes (right) in a sample obtained from a non-diabetic control subject. Glucose uptake was monitored using a fluorescent glucose conjugate internalization assay.

FIG. 2D is a graph comparing the glucose uptake of BCG-treated monocytes from a non-diabetic control (left) and BCG-treated monocytes (right) from a type 1 diabetic subject. Glucose uptake was monitored using a fluorescent glucose conjugate internalization assay.

FIG. 2E is a graph comparing the glucose uptake of untreated monocytes from a non-diabetic control (left) and untreated monocytes (right) from a type 1 diabetic subject. Glucose uptake was monitored using a fluorescent glucose conjugate internalization assay.

FIG. 2F is a graph showing the effect of age of onset (AOO) on the response of type 1 diabetic subjects to BCG treatment. BCG response is shown in terms of glucose uptake, as assessed by way of a fluorescent glucose conjugate internalization assay.

FIG. 2G is a graph showing the effect of BCG on the uptake of glucose in monocytes obtained from a type 2 diabetic subject. Values along the y-axis represent mean fluorescence intensity of cells following incubation with a fluorescent glucose conjugate. Data on the left of the x-axis correspond to untreated monocytes. Data on the right of the x-axis correspond to BCG-treated monocytes.

FIG. 3A is a graph showing responsiveness to BCG vaccines in type 1 diabetic subjects (T1D) with juvenile onset diabetes (age of onset <21 years) using the Tokyo strain over a 2 year period (x-axis). Responsiveness was measured as the percent change from baseline in HbA1c (y-axis) of open label T1D subjects receiving BCG treatment with an age of onset (AOO) of under 21 years old (n=5) (mean AOO of 11±3 years) compared to a T1D reference population not receiving BCG treatment (n=62). The fall in HbA1c is pronounced (p=0.015). The current chronological ages of the two adult groups were for 28±3 years for the T1D subjects. The duration of diabetes in the T1D group was 18±3 years.

FIG. 3B is a graph showing responsiveness to BCG vaccines in subjects with latent autoimmune diabetes in adults (LADA) using the Tokyo strain over a 2 year period (x-axis). Responsiveness was measured as the percent change from baseline in HbA1c (y-axis) of the current open label T1D subjects with age of onset (AOO) >21 years (mean AOO of 31±2 years) receiving Tokyo BCG treatment (n=4) as compared to a T1D reference population not receiving BCG treatment (n=62). The LADA subjects showed no significant difference between the curves (p=0.509). The current chronological ages of the two adults groups were for 28±3 years for the T1D and 45±4 years for LADA subjects. The duration of diabetes in the T1D group was 18±3 years and the duration of diabetes in the LADA group was 19±2 years.

FIG. 3C is a graph showing the percent change from baseline in HbA1c (y-axis) of the current open label T1D subjects with age of onset (AOO)<21 years (mean AOO of 11±3.0 years) receiving Tokyo BCG treatment (n=5) as compared to the previously published Phase 1 Sanofi BCG clinical trial data with similarly early onset (11±5.8 years) (black, open triangle).

FIG. 4A is a series of flow cytometry plots showing gating and glucose uptake measured with 2-NBDG glucose uptake analysis of non-diabetic control (NDC) monocytes treated in vitro with BCG for 24 hours and then allowed to transport 2-NBDG (sugar) for 1 hour.

FIG. 4B is a series of plots showing the glucose uptake (y-axis) in LADA (open dots) and T1D (closed dots) monocytes with and without exposure to BCG. Both LADA and T1D monocytes differ at baseline (left) and after the in vitro BCG exposure (right) with respect to glucose transport. At baseline, T1D monocytes' basal glucose transport was 6804±170 and LADA monocytes' basal glucose transport was 7641±325. After exposure to BCG, the median fluorescence intensity (MFI) was 8331±241 for T1D monocytes and 9299±421 for LADA monocytes (p<0.0001 for both comparisons). At baseline T1 Ds have insufficient glucose transport compared to augmented baseline transport in LADA monocytes.

FIG. 4C is a series of plots showing glucose uptake (y-axis) in cultured monocytes from NDC (left plot), T1D (middle plot) and LADA (right plot) subjects before and after exposure to BCG. Untreated monocytes exhibit accelerated glucose transport after BCG exposure (P<0.0001 for all comparisons).

FIG. 5 shows glucose uptake measured with 2-NBDG glucose uptake analysis (y-axis) of adult bladder cancer subjects treated with the TICE BCG strain. Glucose uptake in monocytes of non-diabetic control (NDC) subjects (n=23) following a one day incubation with or without BCG in culture compared to three elderly subject receiving high dose BCG for bladder cancer (n=3). One bladder cancer treated subject was studied on two occasions, one bladder cancer subject had type 2 diabetes (T2D) and two of the subjects were NDCs. The sugar uptake is presented as the difference from baseline to stimulated glucose uptake of monocytes in culture with BCG (p=0.0516 (T test one tail, unpaired, Welch's correction)).

FIG. 6A is a plot of glucose uptake (y-axis) in untreated monocytes at baseline from NDC (n=17), T1D (n=43), and T2D (n=13) subjects.

FIG. 6B is a plot of glucose uptake (y-axis) in monocytes from NDC, T1D, and T2D subjects after in vitro treatment with BCG for 24 hours.

FIG. 6C is a comparison of glucose uptake (y-axis) by monocytes from NDC and T2D subjects. Overnight culture in BCG increased glucose uptake in both NDC and T2D monocytes (p<0.05 * p<0.01**; p<0.001***).

FIG. 7A is a plot showing glucose uptake (y-axis) in untreated monocytes from NDC subjects (n=9). Treatment of these monocytes with metformin inhibited glucose uptake as measured by 2-NBDG glucose uptake analysis (p=0.00075).

FIG. 7B is a plot showing glucose uptake (y-axis) in monocytes from NDC subjects cultured in the presence of BCG (n=9). Treatment of these monocytes with metformin inhibited glucose uptake as measured by 2-NBDG glucose uptake analysis (p=0.0047).

FIG. 7C is a plot showing glucose uptake (y-axis) in monocytes from T1D subjects (n=18). Treatment of these monocytes with metformin inhibited glucose uptake as measured by 2-NBDG glucose uptake analysis in T1D monocytes (p=0.0014).

FIG. 7D is a plot showing glucose uptake (y-axis) in monocytes from T1D subjects cultured in the presence of BCG (n=18). Treatment of these monocytes with metformin inhibited glucose uptake as measured by 2-NBDG glucose uptake analysis (p=3.11·10⁻⁶).

FIG. 7E is a plot showing glucose uptake (y-axis) in monocytes from T2D subjects (n=6). Treatment of these monocytes with metformin inhibited glucose uptake as measured by 2-NBDG glucose uptake analysis (p=0.014).

FIG. 7F is a plot showing glucose uptake (y-axis) in monocytes from T2D subjects cultured in the presence of BCG (n=6). Treatment of these monocytes with metformin inhibited glucose uptake as measured by 2-NBDG glucose uptake analysis (p=0.0012).

FIG. 8A is a Kaplan Meyer survival plot for NOD mice that were untreated (CTRL), treated with BCG only (+BCG), treated with metformin only (+Met), or treated with metformin and BCG (+Met and +BCG). Metformin treatment started at 6 weeks of age in vivo; BCG injections were administered at week 7 and week 13. Survival was improved after treatment with BCG, but treatment with metformin alone or with a combination of metformin plus BCG resulted in reduced animal survival due to severe hyperglycemia.

FIG. 8B shows glucose uptake (as measured by 2-NBDG glucose uptake analysis) in isolated bone marrow cells from untreated, BCG-treated, and metformin-treated diabetic NOD mice. These results show significant differences between untreated and BCG-treated mice (p=0.015), as well as between BCG-treated and metformin treated mice (p=0.011). In all cases metformin was started at 6 weeks of age and this was then followed by BCG treatment at week 7 and week 13. Number of mice: NOD n=16; NOD+BCG n=11; NOD+metformin n=15.

FIG. 8C shows the change in blood sugar (y-axis) with a BCG treatment regimen for obese db/db mice (a model of T2D). The arrows indicate BCG treatment times. The BCG-treated mice have substantially lower blood sugars. Open symbols depict timepoints where the difference between CTRL and BCG groups was significant in Student's t testing.

FIG. 8D shows the change in body weight (y-axis) in the severely obese db/db with the BCG treatment regimen.

FIG. 8E is a bar graph of in vivo BCG treatment of normal BALB/c mice and of diabetic db/db mice. Both treatments resulted in increased glucose uptake although the Balb/c mouse effect did not reach statistical significance as measured by 2-NBDG assays in bone marrow cells compared to untreated control mice. BALB/c: n=4 BCG and n=4 untreated; p=0.31; db/db: n=10 BCG and n=10 untreated; p=0.04.

### DETAILED DESCRIPTION

Described herein are compositions and methods for treating a subject (e.g., a mammalian subject, such as a human subject) with diabetes (e.g., type 1 diabetes) by administration of Bacillus Calmette-Guerin (BCG). The compositions and methods of the disclosure can also be used to determining the likelihood that a subject with diabetes (e.g., type 1 diabetes) will respond to treatment with BCG. Thus, using the compositions and methods of the disclosure, a subject with diabetes may be tested for one or more of a variety of attributes in order to assess the subject's propensity to respond to BCG therapy. A subject determined to be responsive to BCG treatment, using the methods described herein, may be subsequently treated with BCG, according to the methods described herein.

The compositions and methods of the disclosure are based, in part, on the discovery that a subject (e.g., a human subject) with diabetes is more likely to benefit from BCG if the subject exhibits particular characteristics prior to administration of BCG. For example, a subject (e.g., a human subject) with diabetes is more likely to respond to BCG treatment if the subject is less than 40 years old at the time of onset of the diabetes, such 39 years old, 38 years old, 37 years old, 36 years old, 35 years old, 34 years old, 33 years old, 32 years old, 31 years old, 30 years old, 29 years old, 28 years old, 27 years old, 26 years old, 25 years old, 24 years old, 23 years old, 22 years old, 21 years old, 20 years old, 19 years old, 18 years old, 17 years old, 16 years old, 15 years old, 14 years old, 13 years old, 12 years old, 11 years old, 10 years old, 9 years old, 8 years old, 7 years old, 6 years old, 5 years old, 4 years old, or 3 years, or less, at the time of onset of the diabetes. Surprisingly, a subject's chronological age and the duration of time that the subject has been living with diabetes are not determinative of the likelihood that the subject will respond to BCG treatment. This discovery leads to a series of clinical benefits. Using the compositions and methods of the disclosure, a subject may be identified as likely to respond to BCG, and may be treated accordingly, if the subject is determined to be less than 40 years old (e.g., 39 years old, 38 years old, 37 years old, 36 years old, 35 years old, 34 years old, 33 years old, 32 years old, 31 years old, 30 years old, 29 years old, 28 years old, 27 years old, 26 years old, 25 years old, 24 years old, 23 years old, 22 years old, 21 years old, 20 years old, 19 years old, 18 years old, 17 years old, 16 years old, 15 years old, 14 years old, 13 years old, 12 years old, 11 years old, 10 years old, 9 years old, 8 years old, 7 years old, 6 years old, 5 years old, 4 years old, or 3 years, or less) at the time of onset of the diabetes, regardless of the chronological age of the subject and regardless of the duration of time that the subject has been living with diabetes at the time the subject is treated with a composition comprising BCG, according to the methods described herein. This discovery facilitates the treatment of subjects that, for example, have been living with diabetes (e.g., type 1 diabetes) for a long period of time (and for whom treatment was thought not possible). For example, low circulating levels of endogenous c-peptide, such as c-peptide levels of from 0.1 pM to 5 pM, are indicative of little to no pancreatic beta islet cell activity, and thus, little to no secretion of endogenous insulin. Even a subject in this severe of a diabetic state may be treated with BCG using the compositions and methods of the disclosure, provided, for example, that the subject was 40 years old or younger at the time of onset of the diabetes (e.g., 39 years old, 38 years old, 37 years old, 36 years old, 35 years old, 34 years old, 33 years old, 32 years old, 31 years old, 30 years old, 29 years old, 28 years old, 27 years old, 26 years old, 25 years old, 24 years old, 23 years old, 22 years old, 21 years old, 20 years old, 19 years old, 18 years old, 17 years old, 16 years old, 15 years old, 14 years old, 13 years old, 12 years old, 11 years old, 10 years old, 9 years old, 8 years old, 7 years old, 6 years old, 5 years old, 4 years old, or 3 years, or less at the time of onset of the diabetes).

Additionally or alternatively, using the compositions and methods of the disclosure, a subject with diabetes (e.g., type 1 diabetes) may be assessed for their likelihood to benefit from BCG therapy on the basis of the subject's pre-treatment level of glucose uptake. For example, a subject that exhibits a lower rate of glucose uptake (e.g., in peripheral blood cells, such as leukocytes, including, e.g., monocytes) relative to a reference rate of glucose uptake, such as a rate of glucose uptake exhibited by a subject that does not have diabetes, is more likely to benefit from BCG treatment. Thus, using the compositions and methods described herein, a subject may be tested for their likelihood to benefit from BCG therapy by measuring a rate of glucose uptake exhibited by the subject and comparing this rate to a reference rate of glucose uptake (e.g., a rate of glucose uptake exhibited by a subject that does not have diabetes, such as a rate of glucose uptake exhibited in peripheral blood cells, such as leukocytes, including, e.g., monocytes, in a subject that does not have diabetes). In this assay, a finding that the subject exhibits a reduced level of glucose uptake relative to such a reference level (e.g., a level of glucose uptake that is reduced by 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50%, or more, relative to a reference level) indicates that the subject is likely to respond to BCG treatment. Exemplary glucose uptake assays are described in Zou et al., Journal of Biochemical and Biophysical Methods 64:207-215 (2005), the disclosure of which is incorporated by reference herein; see also Example 1, below.

Additionally or alternatively, using the compositions and methods of the disclosure, a subject with diabetes (e.g., type 1 diabetes) may be determined to be particularly likely to benefit from BCG therapy if the subject has an overactive (i.e., higher) rate of oxidative phosphorylation relative to a reference rate of oxidative phosphorylation (e.g., a reference rate of from about 25 pmol of O₂ consumption per minute to about 100 pmol of O₂ consumption per minute) and/or a reduced rate of aerobic glycolysis relative to a reference rate of aerobic glycolysis (e.g., a reference rate of from about 5 mpH units per minute to about 50 mpH units per minute). Accordingly, using the compositions and methods described herein, a subject may be tested for their propensity to benefit from BCG therapy by determining a rate of oxidative phosphorylation and/or aerobic glycolysis exhibited by the subject. These quantities may then be compared to a reference rate of oxidative phosphorylation and/or aerobic glycolysis (e.g., a rate of oxidative phosphorylation and/or aerobic glycolysis exhibited by a subject that does not have diabetes). In this context, a finding that the subject exhibits an elevated rate of oxidative phosphorylation (e.g., a rate of oxidative phosphorylation that is increased by 5%, %, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 2%, 93%, 94%, 95%, 96%, 97%, 98%, 99%, 100%, 200%, or more, relative to a reference rate of oxidative phosphorylation (e.g., a reference rate of from about 25 pmol of O₂ consumption per minute to about 100 pmol of O₂ consumption per minute)) and/or a reduced rate of aerobic glycolysis (e.g., a level of aerobic glycolysis that is reduced by 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50%, or more, relative to a reference rate of aerobic glycolysis (e.g., a reference rate of from about 5 mpH units per minute to about 50 mpH units per minute) identifies the subject as one that is particularly likely to benefit from BCG treatment.

Additionally or alternatively, using the compositions and methods of the disclosure, one may assess a subject's likelihood of benefitting from BCG treatment by observing the effect that BCG has on glucose uptake, oxidative phosphorylation, and/or aerobic glycolysis in a sample of cells obtained from the subject. For example, a sample of cells (e.g., peripheral blood cells, such as leukocytes, including, e.g., monocytes) obtained from the subject may be incubated in the presence and absence of BCG. An observation of an increase in the rate of glucose uptake by the cells (e.g., by 5%, %, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 2%, 93%, 94%, 95%, 96%, 97%, 98%, 99%, 100%, 200%, or more), a decrease in the rate of oxidative phosphorylation in the cells (e.g., by 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50%, or more), and/or an increase in the rate of aerobic glycolysis in the cells (e.g., by 5%, %, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 2%, 93%, 94%, 95%, 96%, 97%, 98%, 99%, 100%, 200%, or more) following exposure to the BCG identifies the subject as one that is particularly likely to benefit from treatment with BCG. Thus, these assays facilitate the identification of a subject as likely to benefit from BCG therapy and to be appropriately selected for such treatment.

The sections that follow describe in further detail the characteristics that can identify a subject as likely to benefit from BCG treatment, as well as exemplary assays that can be used in order to measure these characteristics.

### Age of Onset of Diabetes

Using the compositions and methods of the disclosure, a subject (e.g., a human subject) with diabetes (e.g., type 1 diabetes) may be identified as likely to benefit from BCG therapy if the subject is determined to have been less than 40 years old (e.g., 39 years old, 38 years old, 37 years old, 36 years old, 35 years old, 34 years old, 33 years old, 32 years old, 31 years old, 30 years old, 29 years old, 28 years old, 27 years old, 26 years old, 25 years old, 24 years old, 23 years old, 22 years old, 21 years old, 20 years old, 19 years old, 18 years old, 17 years old, 16 years old, 15 years old, 14 years old, 13 years old, 12 years old, 11 years old, 10 years old, 9 years old, 8 years old, 7 years old, 6 years old, 5 years old, 4 years old, or 3 years old, or less) at the time of onset of the diabetes. Advantageously, a subject with diabetes (e.g., type 1 diabetes) may be determined to have a high propensity to respond to BCG therapy, and may be treated accordingly, even if the subject currently has a high chronological age, and/or even if the subject has been living with diabetes for an extended period of time. For example, the compositions and methods of the disclosure can be used to effectively treat even a subject that has a low circulating level of endogenous c-peptide, which is a proxy for pancreatic beta islet cell function and endogenous insulin production. Using the compositions and methods described herein, a subject having a c-peptide level of, for example, from 0.1 pM to 5 pM, may be identified as likely to respond to BCG therapy and may be treated accordingly, despite the advanced state of the subject's diabetic condition, provided, for example, that the subject is determined to have been less than 40 years old (e.g., 39 years old, 38 years old, 37 years old, 36 years old, 35 years old, 34 years old, 33 years old, 32 years old, 31 years old, 30 years old, 29 years old, 28 years old, 27 years old, 26 years old, 25 years old, 24 years old, 23 years old, 22 years old, 21 years old, 20 years old, 19 years old, 18 years old, 17 years old, 16 years old, 15 years old, 14 years old, 13 years old, 12 years old, 11 years old, 10 years old, 9 years old, 8 years old, 7 years old, 6 years old, 5 years old, 4 years old, or 3 years old, or less) at the time of onset of the diabetes.

### Glucose Uptake

Using the compositions and methods of the disclosure, a subject (e.g., a human subject) with diabetes (e.g., type 1 diabetes) may be tested for their likelihood of responding to BCG therapy on the basis of the subject's rate of glucose uptake. Particularly, a finding that the subject exhibits a rate of glucose uptake that is less than a reference rate of glucose uptake (e.g., a rate of glucose uptake in a subject that does not have diabetes) identifies the subject as likely to benefit from BCG treatment. Additionally or alternatively, a subject may be tested for their likelihood to benefit from BCG treatment by obtaining a sample of cells from the subject (e.g., a sample of peripheral blood cells, such as a sample of leukocytes, e.g., monocytes) and comparing the rate of glucose uptake in the cells in the presence and absence of BCG. In this context, a finding that the sample of cells exhibits elevated glucose uptake in the presence of BCG as compared to glucose uptake in the absence of BCG identifies the subject as likely to respond to BCG therapy.

Glucose uptake may be observed empirically using a variety of assays. An example of a glucose uptake assay that may be used in conjunction with the compositions and methods of the disclosure is a fluorescent glucose cell internalization assay. In this assay, a fluorescent glucose compound is obtained by conjugating glucose, such as a variant of glucose that has been modified so as to contain one or more reactive chemical substituents, with a fluorophore. The resulting conjugate is then incubated with a sample of cells for a period of time sufficient for the glucose conjugate to be internalized by the cells (e.g., by way of a transport protein, such as GLUT1). Following this incubation step, the cells are then washed to remove glucose conjugate that has not been internalized by the cells. The cells are then subject to a fluorescence analysis. In this setting, fluorescence that is detected at an emission wavelength of the fluorophore correlates with internalization of the fluorescent glucose conjugate by the cells.

### Fluorophores

Fluorescent moieties that may be used in conjunction with the glucose uptake assays of the disclosure include, without limitation, 7-nitrobenz-2-oxa-1,3-diazol-4-yl (NBD), methoxycoumarin, dialkylaminocoumarin, hydroxycoumarin, aminomethylcoumarin acetate (AMCA), 6,8-difluoro-7-hydroxy-3-carboxycoumarin (PACIFIC BLUE™ dye), 5-(dimethylamino)naphthalene-1-sulfonyl (Dansyl), pyrene, 7-amino-3-{[(2,5-dioxopyrrolidin-1-yl)oxy]-2-oxoethyl}-4-methyl-2-oxo-2H-chromene-6-sulfonic acid (ALEXA FLUOR 350™), 6,8-difluoro-7-hydroxy-4-methylcoumarin (MARINA BLUE™ dye), N-(2-aminoethyl)-4-{5-[4-(dimethylamino)phenyl]-1,3-oxazol-2-yl}benzenesulfonamide (DAPOXYL™ dye), 2,3,5,6-Tetramethyl-1H,7H-pyrazolo[1,2-a]pyrazole-1,7-dione (Bimane), 4-{[4-(Diethylamino)phenyl][4-(ethylamino)naphthalen-2-yl]methylidene}-N,N-diethylcyclohexa-2,5-dien-1-iminium (CASCADE BLUE™ dye), tris(N,N-diethylethanaminium) 8-[2-(4-{[(2,5-dioxopyrrolidin-1-yl)oxy]carbonyl}piperidin-1-yl)-2-oxoethoxy]pyrene-1,3,6-trisulfonate (ALEXA FLUOR 405™), N,N-diethylethanaminium [9-{6-[(2,5-dioxopyrrolidin-1-yl)oxy]-6-oxohexyl}-8,8-dimethyl-2-oxo-4-(trifluoromethyl)-8,9-dihydro-2H-benzo[g]chromen-6-yl]methanesulfonate (ALEXA FLUOR 430™), 1-[({4-[(7-nitro-2,1,3-benzoxadiazol-4-yl)amino]phenyl}acetyl)oxy]pyrrolidine-2,5-dione (QSY™ dye), fluorescein, 2-(6-amino-3-iminio-4,5-disulfonato-3H-xanthen-9-yl)-5-((2,3,5,6-tetrafluorophenoxy)carbonyl)benzoate (ALEXA FLUOR 488™) 2′,7′-Difluoro-3′,6′-dihydroxy-3H-spiro[isobenzofuran-1,9′-xanthen]-3-one (OREGON GREEN™ 488), 1,3,5,7,8-pentamethyl-4,4-difluorro-4-bora-3a,4a-diaza-s-indacene (BODIPY™ 493/503), rhodamine green, and rhodamine red. For example, in some embodiments, the fluorescent glucose conjugate is 2-[N-(7-nitrobenz-2-oxa-1,3-diazol-4-yl)amino]-2-deoxy-D-glucose (2-NBDG).

### Linkers for Chemical Conjugation

A variety of linkers can be used to covalently couple reactive substituents within a glucose molecule to a fluorescent dye. Exemplary linkers include those that may be cleaved, for instance, by enzymatic hydrolysis, photolysis, hydrolysis under acidic conditions, hydrolysis under basic conditions, oxidation, disulfide reduction, nucleophilic cleavage, or organometallic cleavage (see, for example, Leriche et al., Bioorg. Med. Chem., 20:571-582, 2012, the disclosure of which is incorporated herein by reference as it pertains to linkers suitable for chemical coupling). Examples of linkers useful for the synthesis of fluorescent glucose conjugates described herein include those that contain electrophiles, such as Michael acceptors (e.g., maleimides), activated esters, electron-deficient carbonyl compounds, and aldehydes, among others, suitable for reaction with nucleophilic substituents present within glucose or a glucose analog, such as amine and thiol moieties. For instance, suitable linkers include, without limitation, alkyl, cycloalkyl, and heterocycloalkyl linkers, such as open-chain ethyl, propyl, butyl, hexyl, heptyl, octyl, nonyl, or decyl chains, cyclohexyl groups, cyclopentyl groups, cyclobutyl groups, cyclopropyl groups, piperidinyl groups, morpholino groups, or others containing two reactive moieties (e.g., halogen atoms, aldehyde groups, ester groups, acyl chloride groups, acyl anhydride groups, tosyl groups, mesyl groups, or brosyl groups, among others, that can be displaced by reactive nucleophilic atoms), aryl or heteroaryl linkers, such as benzyl, napthyl, or pyridyl groups containing two halomethyl groups that can be displaced by reactive nucleophilic atoms. Exemplary linkers include succinimidyl 4-(N-maleimidomethyl)-cyclohexane-L-carboxylate (SMCC), N-succinimidyl iodoacetate (SIA), sulfo-SMCC, m-maleimidobenzoyl-N-hydroxysuccinimidyl ester (MBS), sulfo-MBS, and succinimidyl iodoacetate, among others. Additional linkers include the non-cleavable maleimidocaproyl linkers, which are described by Doronina et al., Bioconjugate Chem. 17:14-24 (2006), the disclosure of which is incorporated herein by reference as it pertains to linkers for chemical conjugation.

Additional linkers through which one component of a conjugate may be bound to another as described herein include linkers that are covalently bound to one component of the conjugate on one end of the linker and, on the other end of the linker, contain a chemical moiety formed from a coupling reaction between a reactive substituent present on the linker and a reactive substituent present within the other component of the conjugate. Exemplary reactive substituents that may be present within a component of the conjugate include, without limitation, hydroxyl moieties, amino moieties, carboxyl moieties, thiol moieties, propargyl moieties, azido moieties, haloaryl moieties (e.g., fluoroaryl), haloheteroaryl moieties (e.g., fluoroheteroaryl), haloalkyl moieties, and haloheteroalkyl moieties. Linkers useful in conjunction with the conjugates described herein include, without limitation, linkers containing chemical moieties formed by coupling reactions as depicted in Table 2, below. Curved lines designate points of attachment to each component of the conjugate.

TABLE 2
Exemplary chemical moieties formed by coupling reactions in the formation of
fluorescent glucose conjugates

| Exemplary Coupling | |
|---|---|
| Reactions | Chemical Moiety Formed by Coupling Reactions |
| [3 + 2] Cycloaddition | 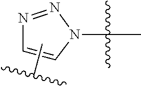 |
| [3 + 2] Cycloaddition | 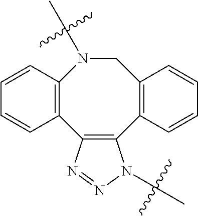 [SMILES: *N1Cc2ccccc2-c2c(nnn2*)-c2ccccc21] |
| [3 + 2] Cycloaddition, Esterification | 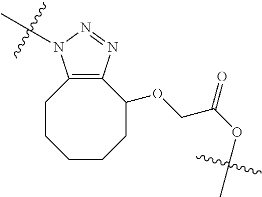 [SMILES: *OC(=O)COC1CCCCCc2c1nnn2*] |
| [3 + 2] Cycloaddition, Esterification | 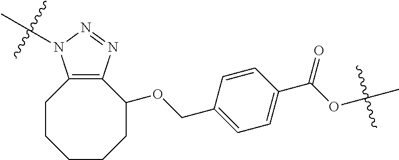 [SMILES: *OC(=O)c1ccc(COC2CCCCCc3c2nnn3*)cc1] |
| [3 + 2] Cycloaddition, Esterification | 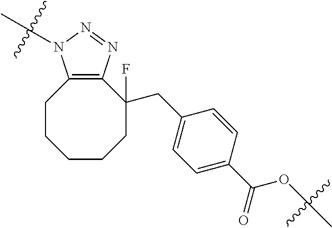 [SMILES: *OC(=O)c1ccc(CC2(F)CCCCCc3c2nnn3*)cc1] |
| [3 + 2] Cycloaddition, Esterification | 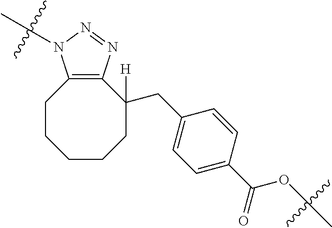 [SMILES: *OC(=O)c1ccc(CC2CCCCCc3c2nnn3*)cc1] |
| [3 + 2] Cycloaddition, Esterification | 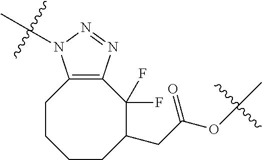 [SMILES: *OC(=O)CC1CCCCc2c(nnn2*)C1(F)F] |
| [3 + 2] Cycloaddition, Esterification | 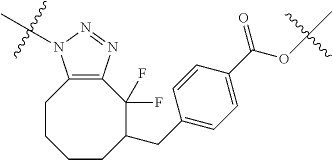 [SMILES: *OC(=O)c1ccc(CC2CCCCc3c(nnn3*)C2(F)F)cc1] |
| [3 + 2] Cycloaddition, Esterification | 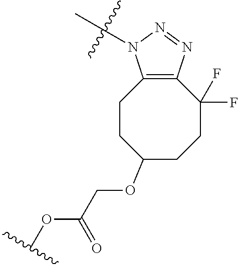 [SMILES: *OC(=O)COC1CCc2c(nnn2*)C(F)(F)CC1] |
| [3 + 2] Cycloaddition, Esterification | 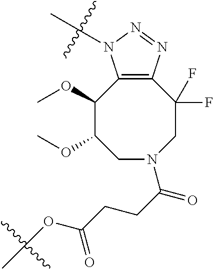 [SMILES: *OC(=O)CCC(=O)N1C[C@H](OC)[C@@H](OC)c2c(nnn2*)C(F)(F)C1] |
| [3 + 2] Cycloaddition, Esterification | 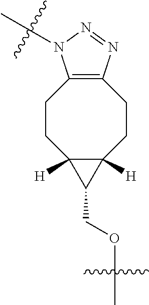 [SMILES: *OC[C@@H]1[C@@H]2CCc3c(nnn3*)CC[C@@H]21] |
| [3 + 2] Cycloaddition, Esterification | 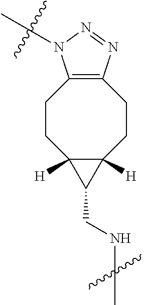 [SMILES: *NC[C@@H]1[C@@H]2CCc3c(nnn3*)CC[C@@H]21] |
| [3 + 2] Cycloaddition, Esterification | 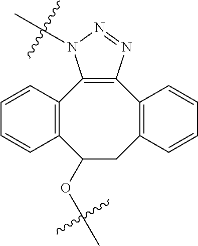 [SMILES: *OC1Cc2ccccc2-c2nnn(*)c2-c2ccccc21] |
| [3 + 2] Cycloaddition, Esterification | 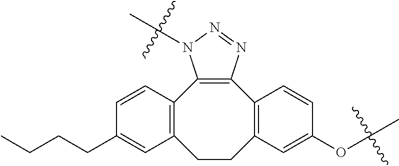 [SMILES: *Oc1ccc2c(c1)CCc1cc(CCCC)ccc1-c1c-2nnn1*] |
| [3 + 2] Cycloaddition | 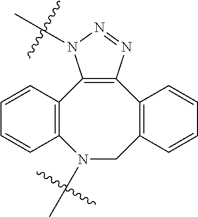 [SMILES: *N1Cc2ccccc2-c2nnn(*)c2-c2ccccc21] |
| Michael addition | 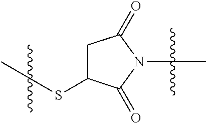 [SMILES: *SC1CC(=O)N(*)C1=O] |
| Michael addition | 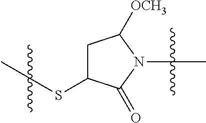 [SMILES: *SC1CC(OC)N(*)C1=O] |
| Imine condensation, Amidation | 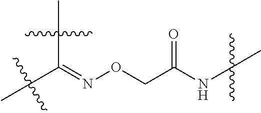 [SMILES: *NC(=O)CON=C(*)*] |
| Imine condensation | 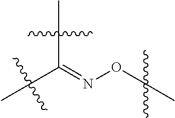 [SMILES: *ON=C(*)*] |
| Disulfide formation | 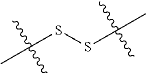 [SMILES: *SS*] |
| Thiol alkylation | 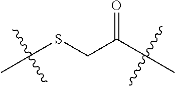 [SMILES: *SCC(*)=O] |
| Condensation, Michael addition | 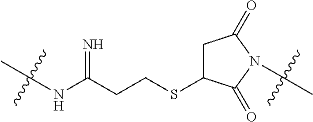 [SMILES: *NC(=N)CCSC1CC(=O)N(*)C1=O] |

### Oxidative Phosphorylation and Aerobic Glycolysis

Using the compositions and methods of the disclosure, a subject (e.g., a human subject) may be identified as likely to benefit from BCG therapy for the treatment of diabetes (e.g., type 1 diabetes) on the basis of a determination that the subject exhibits elevated oxidative phosphorylation and/or reduced aerobic glycolysis relative to a reference rate of oxidative phosphorylation and/or a reference rate of aerobic glycolysis, such as a rate of oxidative phosphorylation and/or a rate of aerobic glycolysis in a healthy subject (e.g., a human subject) that does not have diabetes. Additionally or alternatively, using the compositions and methods of the disclosure, a subject (e.g., a human subject) may be identified as likely to benefit from BCG therapy for the treatment of diabetes (e.g., type 1 diabetes) on the basis of a finding that a sample of cells obtained from the subject exhibits (i) a reduced rate of oxidative phosphorylation in the presence of BCG as compared to the absence of BCG, and/or (ii) an elevated rate of aerobic glycolysis in the presence of BCG as compared to the absence of BCG.

Oxidative phosphorylation and aerobic glycolysis manifest empirically in various ways and may be monitored using assays described herein. For example, oxidative phosphorylation may be observed experimentally by monitoring the rate of oxygen consumption in a sample of cells obtained from a subject (e.g., a human subject) with diabetes (e.g., type 1 diabetes). In this context, an increase in oxygen consumption correlates with an increase in oxidative phosphorylation, as the oxygen used by the cells is consumed for the purpose of producing high-energy covalent bonds, such as the phosphodiester bonds in adenosine trisphosphate or a molecule of similar structure. Similarly, aerobic glycolysis may be monitored experimentally by measuring the rate of extracellular acidification in a sample of cells obtained from a subject (e.g., a human subject) with diabetes (e.g., type 1 diabetes). In this context, an increase in extracellular acidification correlates with an increase in aerobic glycolysis, as the acidification results from an accumulation of pyruvic acid and lactic acid, which are products of glycolysis and ensuing fermentation.

### Dosing Regimens and Routes of Administration

### Dosing Schedules for Administration of BCG

In some embodiments of the disclosure, BCG is administered to a subject (e.g., a human subject, such as a human subject having diabetes (e.g., type 1 diabetes)) in a single dose. Alternatively, BCG may be administered to the subject in multiple doses. For example, using the compositions and methods of the disclosure, BCG may be administered to the subject in one or more doses per day, week, month, or year (e.g., in from 1 to 10 doses per day, week, month, or year, such as in from 1 to 10 doses per year). In some embodiments, the BCG Is administered to the subject in from 1 to 5 doses per year (e.g., in 1, 2, 3, 4, or 5 doses per year).

Using the compositions and methods of the disclosure, BCG may be administered to a subject (e.g., a human subject, such as a human subject having diabetes (e.g., type 1 diabetes)) in a total of from about 2 to about 20 doses (e.g., in 2, 3, 4, 5, 6, 7, 8, 9, 10, 11, 12, 13, 14, 15, 16, 17, 18, 19, or 20 doses) over the course of a treatment period, such as a treatment period having a duration of one or more months or years. In some embodiments, BCG is administered to the subject in a total of from 2 to 10 doses (e.g., in 2, 3, 4, 5, 6, 7, 8, 9, or 10 doses) over the course of a treatment period, such as a treatment period having a duration of one or more months or years. In some embodiments, BCG is administered to the subject in a total of from 2 to 5 doses (e.g., in 2, 3, 4, or 5 doses) over the course of a treatment period, such as a treatment period having a duration of one or more months or years.

For subjects that are administered multiple doses of BCG, the administrations may be evenly spaced, such that there is a uniform amount of time elapsed between each administration, or the administrations may be unevenly spaced, such that there is a different amount of time elapsed between different administrations. In some embodiments, the time elapsed between administration of one dose of BCG and another is from about 1 week to about 12 weeks (e.g., about 1 week, 2 weeks, 3 weeks, 4 weeks, 5 weeks, 6 weeks, 7 weeks, 8 weeks, 9 weeks, 10 weeks, 11 weeks, or 12 weeks). For example, in some embodiments, the time elapsed between administration of one dose of BCG and another is from about 1 day to about 1 year (e.g., about 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, or 52 weeks) or more than 1 year (e.g., from about 2, 3, 4, 5, or 6 years between doses or between treatment periods).

In some embodiments, a time elapsed from administration of one dose of the BCG to administration of a subsequent dose of the BCG is from about 2 months to about 72 months (e.g., about 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, 36 months, 37 months, 38 months, 39 months, 40 months, 41 months, 42 months, 43 months, 44 months, 45 months, 46 months, 47 months, 48 months, 49 months, 50 months, 51 months, 52 months, 53 months, 54 months, 55 months, 56 months, 57 months, 58 months, 59 months, 60 months, 61 months, 62 months, 63 months, 64 months, 65 months, 66 months, 67 months, 68 months, 69 months, 70 months, 71 months, or 72 months). In some embodiments, a time elapsed from administration of one dose of the BCG to administration of a subsequent dose of the BCG is from about 3 months to about 48 months (e.g., about 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, 36 months, 37 months, 38 months, 39 months, 40 months, 41 months, 42 months, 43 months, 44 months, 45 months, 46 months, 47 months, or 48 months). In some embodiments, a time elapsed from administration of one dose of the BCG to administration of a subsequent dose of the BCG is from about 6 months to about 36 months (e.g., about 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, or 36 months). In some embodiments, a time elapsed from administration of one dose of the BCG to administration of a subsequent dose of the BCG is from about 8 months to about 16 months (e.g., about 8 months, 9 months, 10 months, 11 months, 12 months, 13 months, 14 months, 15 months, or 16 months). In some embodiments, a time elapsed from administration of one dose of the BCG to administration of a subsequent dose of the BCG is about 12 months.

Using the compositions and methods of the disclosure, BCG may be administered to a subject (e.g., a human subject having diabetes (e.g., type 1 diabetes)) in accordance with a multi-dose schedule. For example, BCG may be administered to the subject in first, second, third dose, fourth dose, fifth dose, and/or sixth doses, or more. The doses may be arranged, for example, such that:
a) the second dose of the BCG is administered to the subject from about 1 day to about 1 year after administration of the first dose of the BCG to the subject;
b) the third dose of the BCG is administered to the subject from about 2 months to about 72 months after administration of the second dose of the BCG to the subject;
d) the fourth dose of the BCG is administered to the subject from about 2 months to about 72 months after administration of the third dose of the BCG to the subject;
e) the fifth dose of the BCG is administered to the subject from about 2 months to about 72 months after administration of the fourth dose of the BCG to the subject; and
f) the sixth dose of the BCG is administered to the subject from about 2 months to about 72 months after administration of the fifth dose of the BCG to the subject.

### Insulin Therapy

In some embodiments of the disclosure, prior to administration of BCG to a subject (e.g., a human subject) having diabetes (e.g., type 1 diabetes), the subject may be receiving insulin therapy in order to maintain glucose homeostasis. For example, the subject may have such a severe diabetic state (as evidenced, e.g., by a finding that the subject has a circulating level of endogenous c-peptide of from about 0.1 pM to about 5 pM) that the subject may be dependent upon insulin therapy to survive. The insulin may be, for example, produced recombinantly (e.g., by way of expression from a vector containing a nucleic acid encoding insulin, using molecular biology techniques known in the art) or synthesized chemically (e.g., by way of solid-phase peptide synthesis techniques known in the art). Exemplary insulin agents that may be administered to the subject as part of the insulin therapy include, without limitation, insulin lispro injection (ADMELOG™), insulin glulisine (APIDRA™), insulin aspart (FIASP™), insulin lispro (HUMALOG™), human insulin isophane suspension (HUMULIN N™), insulin glargine (LANTUS™) insulin detemir (LEVEMIR™), insulin degludec (TRESIBA FLEXTOUCH™), 75% insulin lispro protamine suspension/25% insulin lispro injection (HUMALOG MIX 75/25™), 70% human insulin isophane suspension/30% human insulin injection (HUMALOG 70/30™), 50% insulin lispro protamine suspension/50% insulin lispro injection (HUMALOG MIX 50/50™), 70% insulin aspart protamine suspension/30% insulin aspart injection (NOVOLOG MIX 70/30™), 70% insulin degludec/30% insulin aspart (RYZODEG 70/30 FLEXTOUCH™), and 70% NPH human insulin/30% regular human insulin injection (HUMULIN 70/30™). Additional examples of insulin therapeutics include variants of the foregoing insulin polypeptides, such as polypeptides having one or more conservative amino acid substitutions relative to the foregoing insulin polypeptides and/or polypeptides having an amino acid sequence that has at least 85% sequence identity (e.g., a sequence identity of 85%, 86%, 87%, 88%, 89%, 90%, 91%, 92%, 93%, 94%, 95%, 96%, 97%, 98%, 99%, or more) to the amino acid sequence of any of the foregoing insulin polypeptides.

In some embodiments of the disclosure, following administration of the BCG to the subject, the frequency of the insulin therapy is reduced. For example, prior to administration of the BCG to the subject, the subject may be receiving insulin in one or more doses (e.g., 5 to 100 units (one unit is about 34.7 μg of pure crystalline insulin) of insulin per dose) per day, week, or month, such as in one or more doses per day, from 2 to 10 doses per week, or from 5 to 25 doses per month. Following administration of the BCG to the subject, the frequency of the insulin therapy may be reduced, for example, such that the subject only receives from 1 to 5 doses of insulin per week or from 2 to 10 doses of insulin per month.

Additionally or alternatively, following administration of the BCG to the subject, the quantity of insulin that is administered to the subject per dose is reduced. For example, the subject may be receiving insulin an amount of from about 0.1 to about 0.2 units/kg prior to administration of the BCG to the subject. Following administration of the BCG to the subject, the subject may receive insulin in a reduced quantity per dose, such as an amount of from about 0.01 to about 0.05 units/kg.

In some embodiments, following administration of the BCG to the subject, the insulin therapy is ceased altogether.

### Routes of Administration

Using the compositions and methods of the disclosure, BCG can be administered to a subject (e.g., a mammalian subject, such as a human) by a variety of routes. For example, BCG may be administered to a subject intradermally, subcutaneously, orally, transdermally, intranasally, intravenously, intramuscularly, intraocularly, parenterally, intrathecally, or intracerebroventricularly (e.g., intradermally or subcutaneously). The most suitable route for administration in any given case will depend on the nature and severity of the particular disease being treated, the subject, pharmaceutical formulation methods, the subject's age, body weight, sex, the subject's diet, and the subject's excretion rate.

Therapeutic formulations of BCG can be administered with medical devices known in the art. For example, a therapeutic formulation of BCG can be administered with a needleless hypodermic injection device, such as the devices disclosed in U.S. Pat. Nos. 5,399,163; 5,383,851; 5,312,335; 5,064,413; 4,941,880; 4,790,824; and 4,596,556; the disclosures of each of which are incorporated herein by reference. Examples of well-known implants and modules useful in conjunction with the compositions and methods of the disclosure include those described in U.S. Pat. No. 4,487,603; which discloses an implantable micro-infusion pump for dispensing medication at a controlled rate; U.S. Pat. No. 4,486,194; which discloses a therapeutic device for administering medicaments through the skin; U.S. Pat. No. 4,447,233; which discloses a medication infusion pump for delivering medication at a precise infusion rate; U.S. Pat. No. 4,447,224; which discloses a variable flow implantable infusion apparatus for continuous drug delivery; U.S. Pat. No. 4,439,196; which discloses an osmotic drug delivery system having multi-chamber compartments; and U.S. Pat. No. 4,475,196; which discloses an osmotic drug delivery system. These patents are incorporated herein by reference. Other such implants, delivery systems, and modules are known to those skilled in the art.

### Pharmaceutical Compositions

Therapeutic compositions containing BCG can be prepared, e.g., using methods known in the art or described herein. For instance, BCG formulations can be prepared using physiologically acceptable carriers, excipients, and/or stabilizers (Remington's Pharmaceutical Sciences 16th edition, Osol, A. Ed. (1980); the disclosure of which is incorporated herein by reference), and in a desired form, e.g., in the form of aqueous solutions or suspensions. The compositions can also be prepared so as to contain BCG at a desired concentration or cell count. BCG compositions of the disclosure also include lyophilized compositions that can be rehydrated prior to administration. The sections that follow describe useful additives that can be included in a BCG formulation for administration to a subject or for long-term storage.

### Strains of BCG

A variety of strains of BCG may be used in conjunction with the compositions and methods of the disclosure. Exemplary strains of BCG include those that can be cultured under good manufacturing protocols, such as the Pasteur, Phipps, Frappier, Mexico, Birkhaug, Sweden, Moreau, Japan-Tokyo, Copenhagen, TICE, Sanofi, Aventis, Connaught, RIVM, Russian, Evans, MMC, Moreau, and Glaxo substrains of BCG, among others, as well as genetic variants of these substrains. Mycobacteria that may be used in conjunction with the compositions and methods of the disclosure may be live, attenuated, or inactivated such that the bacteria retain certain antigen expression patterns but are no longer virulent.

### Unit Dosage Forms of BCG

Exemplary unit dosage forms of BCG useful in conjunction with the compositions and methods of the disclosure include unit dosage forms that contain from about 1×10⁴ colony forming units (CFU) per 0.1 mg of BCG to about 1×10⁸ CFU per 0.1 mg of BCG (e.g., about 1×10⁴ CFU per 0.1 mg of BCG, 2×10⁴ CFU per 0.1 mg of BCG, 3×10⁴ CFU per 0.1 mg of BCG, 4×10⁴ CFU per 0.1 mg of BCG, 5×10⁴ CFU per 0.1 mg of BCG, 6×10⁴ CFU per 0.1 mg of BCG, 7×10⁴ CFU per 0.1 mg of BCG, 8×10⁴ CFU per 0.1 mg of BCG, 9×10⁴ CFU per 0.1 mg of BCG, 1×10⁵ CFU per 0.1 mg of BCG, 2×10⁵ CFU per 0.1 mg of BCG, 3×10⁵ CFU per 0.1 mg of BCG, 4×10⁵ CFU per 0.1 mg of BCG, 5×10⁵ CFU per 0.1 mg of BCG, 6×10⁵ CFU per 0.1 mg of BCG, 7×10⁵ CFU per 0.1 mg of BCG, 8×10⁵ CFU per 0.1 mg of BCG, 9×10⁵ CFU per 0.1 mg of BCG, 1×10⁶ CFU per 0.1 mg of BCG, 2×10⁶ CFU per 0.1 mg of BCG, 3×10⁶ CFU per 0.1 mg of BCG, 4×10⁶ CFU per 0.1 mg of BCG, 5×10⁶ CFU per 0.1 mg of BCG, 6×10⁶ CFU per 0.1 mg of BCG, 7×10⁶ CFU per 0.1 mg of BCG, 8×10⁶ CFU per 0.1 mg of BCG, 9×10⁶ CFU per 0.1 mg of BCG, 1×10⁷ CFU per 0.1 mg of BCG, 2×10⁷ CFU per 0.1 mg of BCG, 3×10⁷ CFU per 0.1 mg of BCG, 4×10⁷ CFU per 0.1 mg of BCG, 5×10⁷ CFU per 0.1 mg of BCG, 6×10⁷ CFU per 0.1 mg of BCG, 7×10⁷ CFU per 0.1 mg of BCG, 8×10⁷ CFU per 0.1 mg of BCG, 9×10⁷ CFU per 0.1 mg of BCG, or 1×10⁸ CFU per 0.1 mg of BCG).

For example, in some embodiments, a unit dosage form of the disclosure contains from about 1×10⁵ CFU per 0.1 mg of BCG to about 1×10⁷ CFU per 0.1 mg of BCG (e.g., about 1×10⁵ CFU per 0.1 mg of BCG, 2×10⁵ CFU per 0.1 mg of BCG, 3×10⁵ CFU per 0.1 mg of BCG, 4×10⁵ CFU per 0.1 mg of BCG, 5×10⁵ CFU per 0.1 mg of BCG, 6×10⁵ CFU per 0.1 mg of BCG, 7×10⁵ CFU per 0.1 mg of BCG, 8×10⁵ CFU per 0.1 mg of BCG, 9×10⁵ CFU per 0.1 mg of BCG, 1×10⁶ CFU per 0.1 mg of BCG, 2×10⁶ CFU per 0.1 mg of BCG, 3×10⁶ CFU per 0.1 mg of BCG, 4×10⁶ CFU per 0.1 mg of BCG, 5×10⁶ CFU per 0.1 mg of BCG, 6×10⁶ CFU per 0.1 mg of BCG, 7×10⁶ CFU per 0.1 mg of BCG, 8×10⁶ CFU per 0.1 mg of BCG, 9×10⁶ CFU per 0.1 mg of BCG, or 1×10⁷ CFU per 0.1 mg of BCG).

In some embodiments, a unit dosage form of the disclosure contains from about 1×10⁶ CFU per 0.1 mg of BCG to about 9×10⁶ CFU per 0.1 mg of BCG (e.g., about 1×10⁶ CFU per 0.1 mg of BCG, 2×10⁶ CFU per 0.1 mg of BCG, 3×10⁶ CFU per 0.1 mg of BCG, 4×10⁶ CFU per 0.1 mg of BCG, 5×10⁶ CFU per 0.1 mg of BCG, 6×10⁶ CFU per 0.1 mg of BCG, 7×10⁶ CFU per 0.1 mg of BCG, 8×10⁶ CFU per 0.1 mg of BCG, or 9×10⁶ CFU per 0.1 mg of BCG).

In some embodiments, a unit dosage form of the disclosure contains from about 1.8×10⁶ CFU per 0.1 mg of BCG to about 3.9×10⁶ CFU per 0.1 mg of BCG (e.g., about 1×10⁶ CFU per 0.1 mg of BCG, 1.1×10⁶ CFU per 0.1 mg of BCG, 1.2×10⁶ CFU per 0.1 mg of BCG, 1.3×10⁶ CFU per 0.1 mg of BCG, 1.4×10⁶ CFU per 0.1 mg of BCG, 1.5×10⁶ CFU per 0.1 mg of BCG, 1.6×10⁶ CFU per 0.1 mg of BCG, 1.7×10⁶ CFU per 0.1 mg of BCG, 1.8×10⁶ CFU per 0.1 mg of BCG, 1.9×10⁶ CFU per 0.1 mg of BCG, 2×10⁶ CFU per 0.1 mg of BCG, 2.1×10⁶ CFU per 0.1 mg of BCG, 2.2×10⁶ CFU per 0.1 mg of BCG, 2.3×10⁶ CFU per 0.1 mg of BCG, 2.4×10⁶ CFU per 0.1 mg of BCG, 2.5×10⁶ CFU per 0.1 mg of BCG, 2.6×10⁶ CFU per 0.1 mg of BCG, 2.7×10⁶ CFU per 0.1 mg of BCG, 2.8×10⁶ CFU per 0.1 mg of BCG, 2.9×10⁶ CFU per 0.1 mg of BCG, 3×10⁶ CFU per 0.1 mg of BCG, 3.1×10⁶ CFU per 0.1 mg of BCG, 3.2×10⁶ CFU per 0.1 mg of BCG, 3.3×10⁶ CFU per 0.1 mg of BCG, 3.4×10⁶ CFU per 0.1 mg of BCG, 3.5×10⁶ CFU per 0.1 mg of BCG, 3.6×10⁶ CFU per 0.1 mg of BCG, 3.7×10⁶ CFU per 0.1 mg of BCG, 3.8×10⁶ CFU per 0.1 mg of BCG, or 3.9×10⁶ CFU per 0.1 mg of BCG).

### Cryopreserved Formulations of BCG

Pharmaceutical compositions of BCG can be prepared for storage by cryopreservation, e.g., by contacting BCG with a cryoprotectant known in the art, such as dimethylsulfoxide (DMSO). Suitable DMSO concentrations in BCG stock solutions range from 0.01% to about 1% DMSO. Cryopreserved solutions can include acceptable carriers, excipients or stabilizers typically employed in the art, e.g., buffering agents, stabilizing agents, preservatives, isotonifiers, non-ionic detergents, antioxidants, and other miscellaneous additives. See, e.g., Remington's Pharmaceutical Sciences, 16th edition (Osol, ed. 1980; incorporated herein by reference). Such additives are generally nontoxic to the subject that is ultimately treated at the dosages and concentrations employed.

### Buffering Agents

A wide array of buffering agents can be included in a BCG formulation useful in conjunction with the methods of the disclosure. These substances serve to maintain the pH of the formulation in a desirable range, e.g., a range that approximates physiological conditions. Buffering agents can be present at concentration ranging from, e.g., about 2 mM to about 50 mM. Suitable buffering agents for use with BCG formulations include both organic and inorganic acids and salts thereof such as citrate buffers (e.g., monosodium citrate-disodium citrate mixture, citric acid-trisodium citrate mixture, citric acid-monosodium citrate mixture, etc.), succinate buffers (e.g., succinic acid-monosodium succinate mixture, succinic acid-sodium hydroxide mixture, succinic acid-disodium succinate mixture, etc.), tartrate buffers (e.g., tartaric acid-sodium tartrate mixture, tartaric acid-potassium tartrate mixture, tartaric acid-sodium hydroxide mixture, etc.), fumarate buffers (e.g., fumaric acid-monosodium fumarate mixture, fumaric acid-disodium fumarate mixture, monosodium fumarate-disodium fumarate mixture, etc.), gluconate buffers (e.g., gluconic acid-sodium glyconate mixture, gluconic acid-sodium hydroxide mixture, gluconic acid-potassium glyuconate mixture, etc.), oxalate buffer (e.g., oxalic acid-sodium oxalate mixture, oxalic acid-sodium hydroxide mixture, oxalic acid-potassium oxalate mixture, etc.), lactate buffers (e.g., lactic acid-sodium lactate mixture, lactic acid-sodium hydroxide mixture, lactic acid-potassium lactate mixture, etc.) and acetate buffers (e.g., acetic acid-sodium acetate mixture, acetic acid-sodium hydroxide mixture, etc.). Additionally, phosphate buffers, histidine buffers and trimethylamine salts such as Tris can be used.

### Preservatives

Preservatives can be added to a formulation of BCG and, optionally, an additional therapeutic agent, in order to retard the growth of other potential microbes in the pharmaceutical composition. For instance, preservatives can be present in a BCG-containing formulation in a wide range of concentrations, e.g., ranging from 0.02%-1% (w/v). Suitable preservatives for use in a pharmaceutical composition of the disclosure include phenol, benzyl alcohol, meta-cresol, methyl paraben, propyl paraben, octadecyldimethylbenzyl ammonium chloride, benzalconium halides {e.g., chloride, bromide, and iodide), hexamethonium chloride, and alkyl parabens such as methyl or propyl paraben, catechol, resorcinol, cyclohexanol, and 3-pentanol. Isotonicifiers can be added to ensure isotonicity of BCG formulations and include polhydric sugar alcohols, for example trihydric or higher sugar alcohols, such as glycerin, arabitol, xylitol, sorbitol and mannitol.

BCG formulations useful in conjunction with the methods of the disclosure may include stabilizers. Stabilizers represent a broad category of excipients which can range in function from a bulking agent to an additive which solubilizes an additional therapeutic agent or helps to prevent denaturation or adherence of the therapeutic agent to the container wall. Typical stabilizers can be polyhydric sugar alcohols (enumerated above); amino acids such as arginine, lysine, glycine, glutamine, asparagine, histidine, alanine, ornithine, L-leucine, 2-phenylalanine, glutamic acid, threonine, etc., organic sugars or sugar alcohols, such as lactose, trehalose, stachyose, mannitol, sorbitol, xylitol, ribitol, myoinisitol, galactitol, glycerol and the like, including cyclitols such as inositol; polyethylene glycol; amino acid polymers; sulfur containing reducing agents, such as urea, glutathione, thioctic acid, sodium thioglycolate, thioglycerol, a-monothioglycerol and sodium thio sulfate; low molecular weight polypeptides (e.g., peptides of 10 residues or fewer); proteins such as human serum albumin, bovine serum albumin, gelatin or immunoglobulins; hydrophylic polymers, such as polyvinylpyrrolidone monosaccharides, such as xylose, mannose, fructose, glucose; disaccharides such as lactose, maltose, sucrose and trisaccharides such as raffinose; and polysaccharides such as dextran. Stabilizers can be present in a BCG formulation in a wide range of concentrations, e.g., from 0.001% to 10.0% (w/w).

### Detergents

Non-ionic surfactants or detergents (also known as “wetting agents”) can be added to help solubilize the therapeutic agent (e.g., an additional therapeutic agent co-formulated with BCG) as well as to protect the therapeutic agent against agitation-induced aggregation, which also permits the formulation to be exposed to shear surface stressed without causing denaturation of a therapeutic protein (e.g., an immunotherapy agent). Suitable non-ionic surfactants that can be added to a formulation containing BCG and, optionally, an additional therapeutic agent include polysorbates (20, 80, etc.), polyoxamers (184, 188 etc.), Pluronic polyols, polyoxyethylene sorbitan monoethers (TWEEN®-20, TWEEN®-80, etc.). Non-ionic surfactants can be present in a range of about 0.05 mg/mL to about 1.0 mg/mL, for example about 0.07 mg/mL to about 0.2 mg/mL. Additional miscellaneous excipients that can be added to a formulation containing BCG and, optionally, an additional therapeutic agent include bulking agents (e.g., starch), chelating agents (e.g., EDTA), antioxidants (e.g., ascorbic acid, methionine, vitamin E), and cosolvents.

### Other Pharmaceutical Carriers

Alternative pharmaceutically acceptable carriers that can be incorporated into a BCG formulation may include dextrose, sucrose, sorbitol, mannitol, starch, rubber arable, potassium phosphate, arginate, gelatin, potassium silicate, microcrystalline cellulose, polyvinylpyrrolidone, cellulose, water, syrups, methyl cellulose, methylhydroxy benzoate, propylhydroxy benzoate, talc, magnesium stearate, and mineral oils. A composition containing BCG may further include a lubricant, an emulsifier, a suspending agent, and a preservative. Details of suitable pharmaceutically acceptable carriers and formulations can be found in Remington's Pharmaceutical Sciences (19th ed., 1995), which is incorporated herein by reference.

### Blood-Brain Barrier Penetration

In certain embodiments, therapeutic agents described herein can be formulated to ensure proper distribution in vivo. For example, the blood-brain barrier (BBB) excludes many high-molecular weight compounds, as well as those with elevated hydrophilicity. To ensure that the therapeutic agents useful with the methods of the disclosure cross the BBB (if desired), they can be formulated, for example, in liposomes. Methods of manufacturing liposomes have been described, e.g., U.S. Pat. Nos. 4,522,811; 5,374,548; and 5,399,331. The liposomes may comprise one or more moieties that are selectively transported into specific cells or organs, thereby enhancing targeted drug delivery (see, e.g., V. V. Ranade, J. Clin. Pharmacol. 29:685, 1989)). Exemplary targeting moieties include, e.g., folate or biotin (see, e.g., U.S. Pat. No. 5,416,016); mannosides (Umezawa et al. (Biochem. Biophys. Res. Commun. 153:1038, 1988)); antibodies (P. G. Bloeman et al. (FEBS Lett. 357:140, 1995); M. Owais et al. (Antimicrob. Agents Chemother. 39:180, 1995)); and surfactant protein A receptor (Briscoe et al. (Am. J. Physiol. 1233:134, 1995)); the disclosures of each of which are incorporated herein by reference.

### EXAMPLES

The following examples are put forth so as to provide those of ordinary skill in the art with a description of how the compositions and methods claimed herein are performed, made, and evaluated, and are intended to be purely exemplary described herein and are not intended to limit the scope of what the inventor regards as her invention.

### Example 1. Predictive Treatment Markers for BCG Therapy in a Diabetes Subject

### Age of Onset and BCG Responsiveness

A series of experiments were conducted to analyze the therapeutic effects of BCG on subjects with type 1 diabetes. Diabetic human subjects were administered BCG and were subsequently monitored to determine the effect of the BCG on the subject's ability to regulate blood glucose. A subject was considered to be highly responsive to BCG treatment if the subject exhibited a reduction in glycated hemoglobin (HbA1c) exceeding 5% from 6 months to 34 months following administration of BCG. The results of these studies are summarized in Table 3, below.

Table 3 shows the effects of three different variables on subjects' responsiveness to BCG: chronological age at the time of monitoring, the subject's duration of the diabetic condition, and the age the subject had at the time of onset of the diabetic condition. Surprisingly, there was no correlation between the subject's chronological age and responsiveness to BCG, nor was there a correlation between the duration of the subject's condition and responsiveness to BCG. Neither of these characteristics determine responsiveness to BCG treatment.

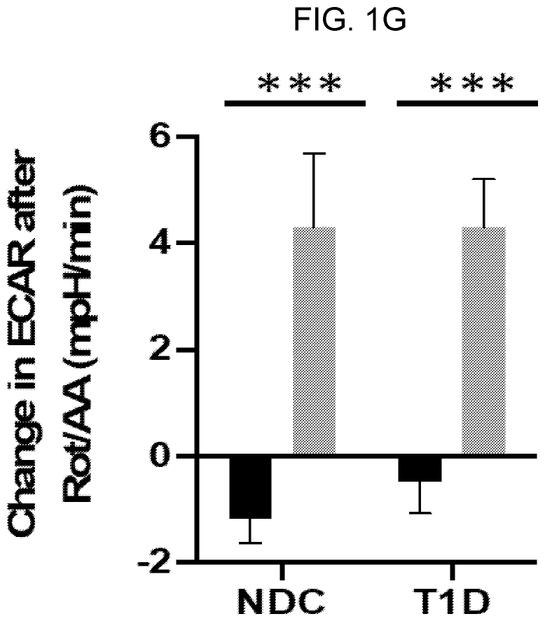

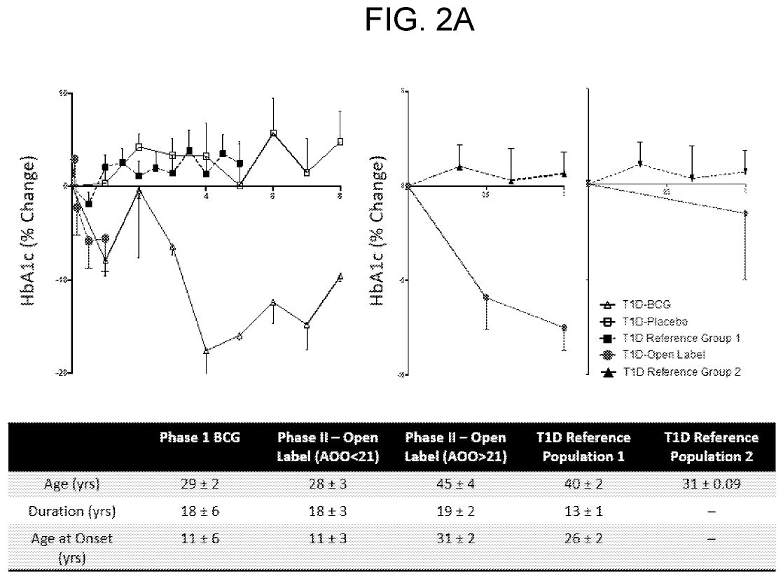

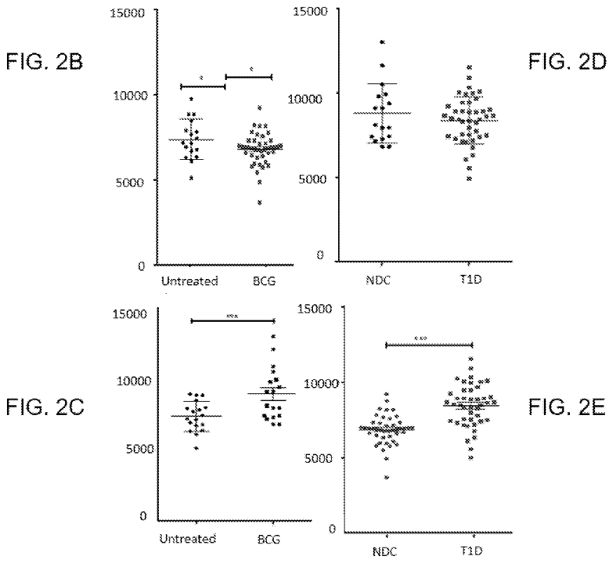

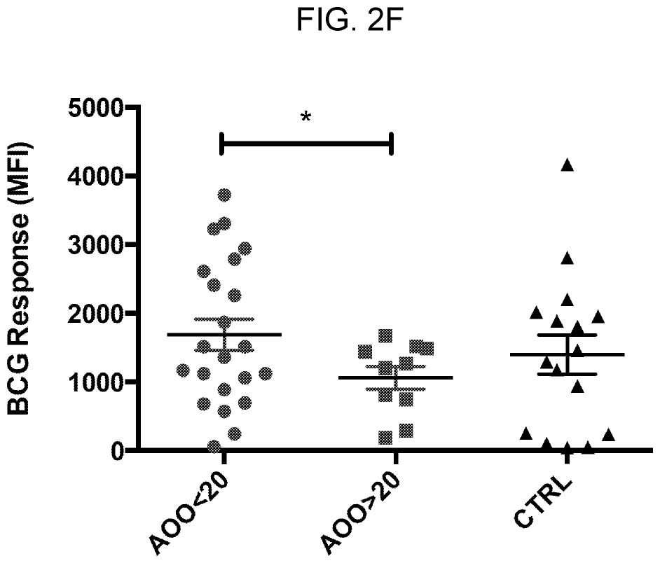

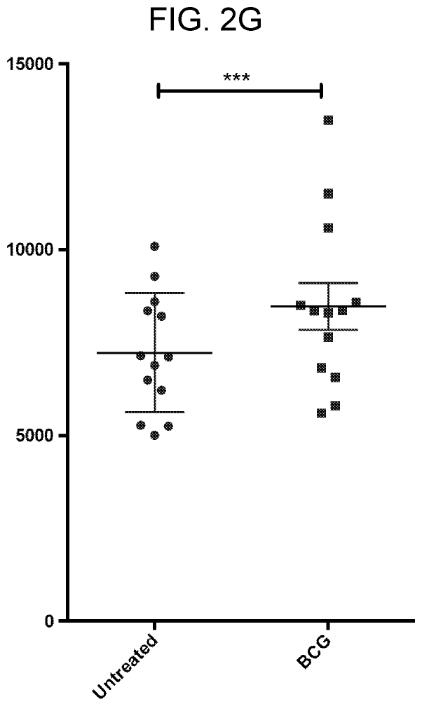

In contrast, there was a statistically significant correlation between the age of the subject at the time of onset of the diabetic condition and the responsiveness of the subject to BCG, as shown below. This trend is illustrated graphically in FIGS. 2A and 2F.

TABLE 3
Effect of age of onset on rapid response to
BCG therapy among type 1 diabetic subjects

| | Highly Responsive | Poorly Responsive | p value |
|---|---|---|---|
| Chronological Age | 38 +/− 15 | 32 +/− 10 | 0.89 |
| Duration of T1D | 20 +/− 9 | 25 +/− 8 | 0.76 |
| Age of onset | 12 +/− 9 | 37 +/− 8 | 0.002 |

Taken together, the data shown in Table 3 demonstrate that a subject (e.g., a human subject) with diabetes (e.g., type 1 diabetes) can be identified as likely to respond to BCG therapy on the basis of the subject's age at the time of onset of the diabetes. Regardless of the current, chronological age of the subject, and regardless of how long the subject has been living with the diabetic condition, the subject can be identified as particularly likely to respond to BCG treatment provided that the age of onset of diabetes in the subject was, for example, 21 years old or younger.

### Glucose Uptake, Oxidative Phosphorylation, and Aerobic Glycolysis as Biomarkers of BCG Responsiveness

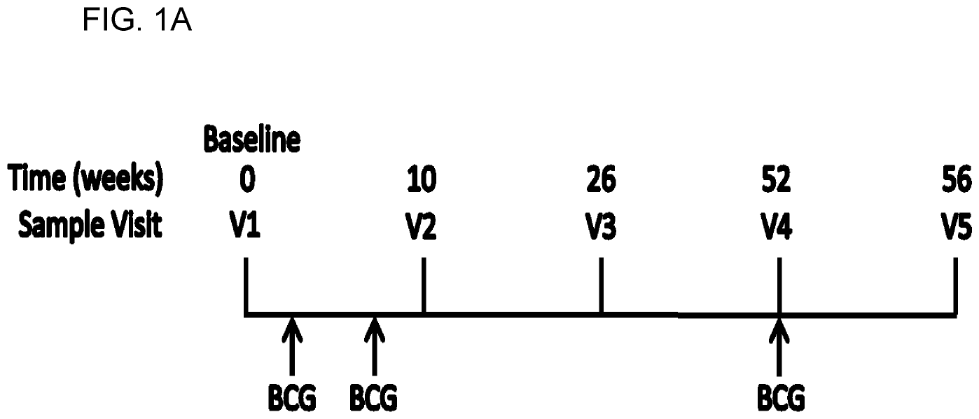

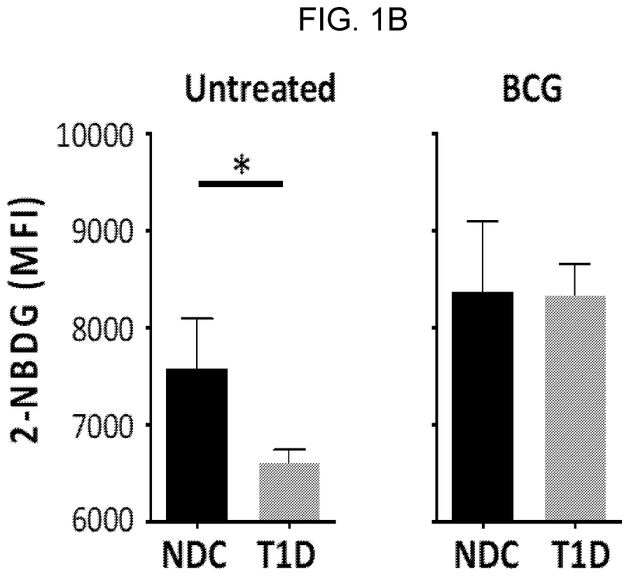

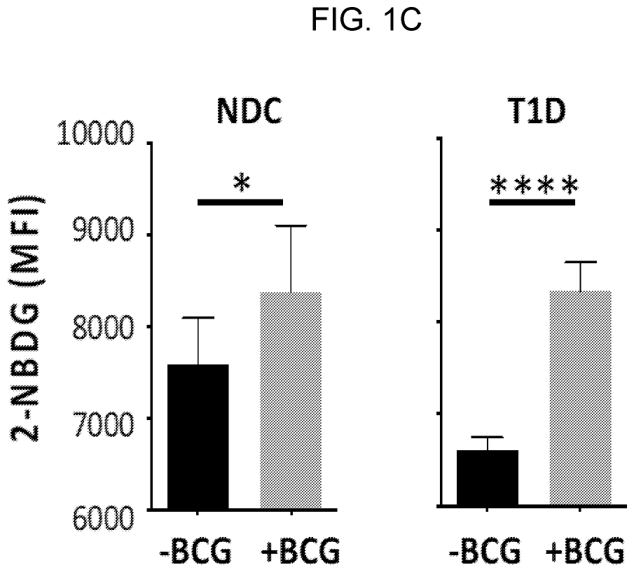

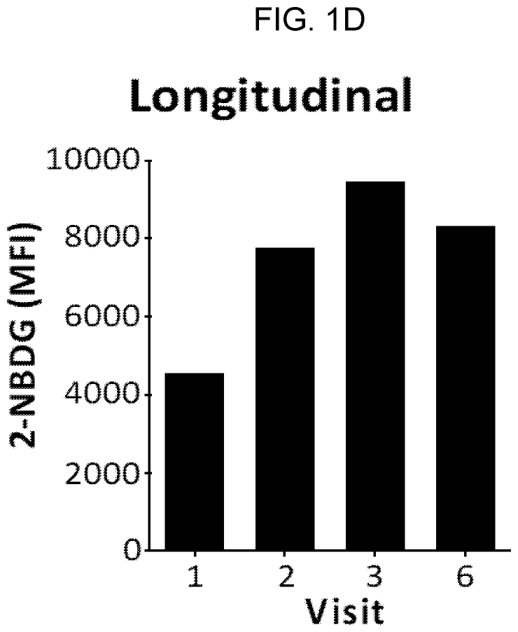

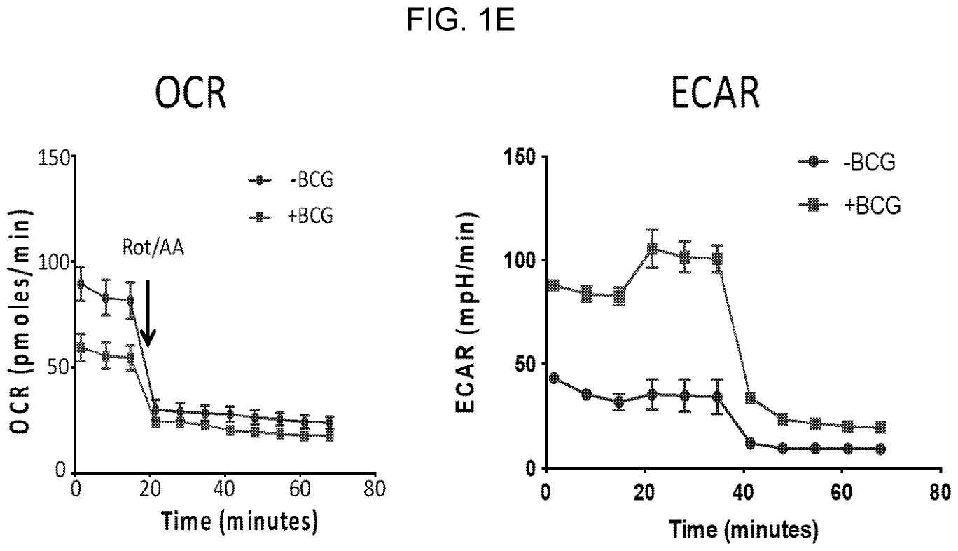

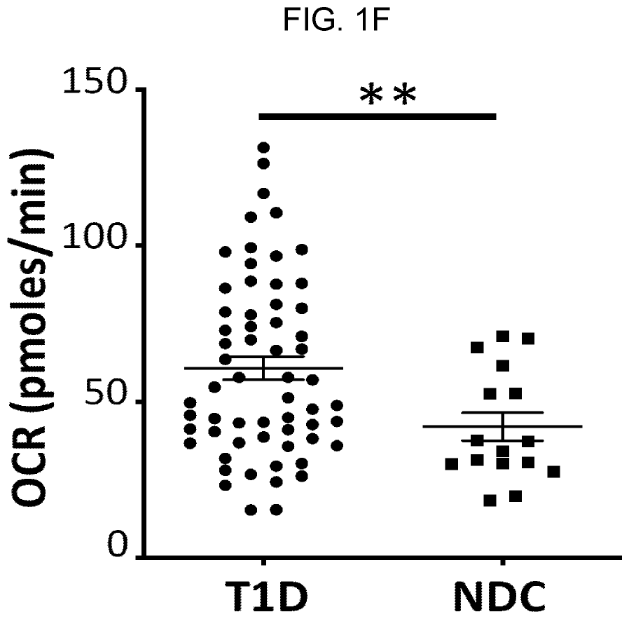

In addition to the findings shown in Table 3, above, the subjects analyzed also demonstrated BCG responsiveness that correlated with an elevated pre-treatment rate of glucose uptake. Subjects were administered BCG according to the treatment schedule shown in FIG. 1A. Following treatment, monocytes were obtained from diabetic subjects and non-diabetic controls. As shown in FIG. 1B, diabetic subjects exhibited a lower rate of glucose uptake relative to non-diabetic controls prior to BCG treatment, as assessed using a fluorescent glucose conjugate internalization assay. Upon incubating monocytes from type 1 diabetics with BCG, the rate of glucose uptake increased (FIG. 1B, right). This observation demonstrates that a subject's responsiveness to BCG therapy in vivo can be predicted based on the glucose uptake response of peripheral blood cells (e.g., monocytes) in a sample taken from the subject in the presence of BCG in vitro. The data in FIG. 1B are presented in a different format in FIG. 1C, which compares the rate of glucose uptake in non-diabetic controls to type 1 diabetes subjects both before and after exposure of monocytes to BCG. In both groups, exposure of monocytes to BCG improved glucose uptake in vitro.

The predictiveness of glucose uptake in vitro on actual subject response in vivo is validated based on the data shown in FIG. 1D. This figure shows the results of a longitudinal study in which a type 1 diabetes subject's glucose uptake rate was monitored following sequential administration of the BCG vaccine. As was the case for monocytes in vitro, the subject exhibited an increase in glucose uptake in vivo following each administration of the BCG vaccine.

Taken together, the data shown in FIGS. 1B-1D demonstrate that BCG exposure to cell samples from a subject in vitro cause an increase in glucose uptake, and that this increase in vitro predicts a corresponding increase in glucose uptake in vivo. FIGS. 2B-2E show the results of additional glucose uptake experiments, and collectively demonstrate that BCG treatment in vitro improves glucose uptake, predictive of a therapeutic response to the BCG vaccine in vivo.

Not only does BCG exert an improvement in glucose uptake in cells of type 1 diabetes subjects, but a similar effect is observed in peripheral blood cells obtained from type 2 diabetics. This is shown in FIG. 2G. As this figure demonstrates, exposure of peripheral blood monocytes from type 2 diabetes subjects to BCG caused a significant increase in glucose uptake. This represents one way in which BCG can be used to achieve glucose regulation even in type 2 diabetes subjects.

In addition to increasing glucose uptake, BCG exposure induces a metabolic shift in peripheral blood cells, causing a decrease in oxidative phosphorylation and an increase in aerobic glycolysis. This result is shown in FIGS. 1E-1G. This shift causes the subject to consume glucose at a significantly more rapid pace, and represents one way in which BCG can help a subject to achieve sustained glucose regulation.

Monocyte isolation and culture and the 2-NBDG sugar transport assay were conducted as described in the materials and methods of Example 13.

### Example 2. Tokyo BCG Strain Therapy in T1D and LADA Subjects

A series of experiments were conducted to analyze the therapeutic effects of the Tokyo BCG strain on subjects with type 1 diabetes (T1D) and latent autoimmune diabetes in adults (LADA), and to see if LADA subjects are equally responsive to the BCG intervention over the same time course as T1D.

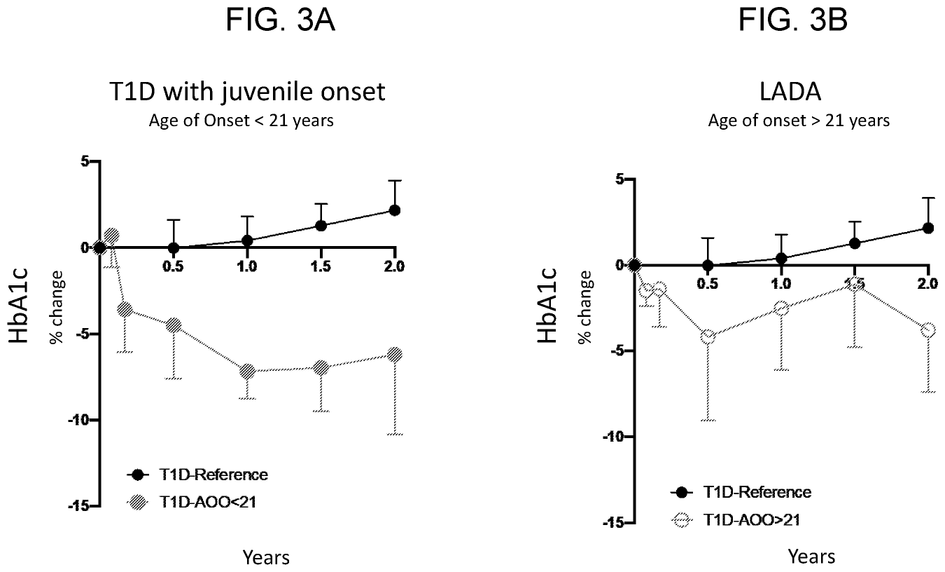

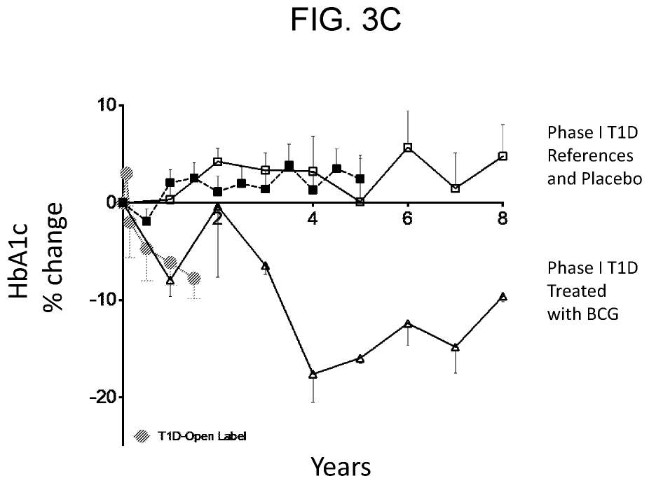

Juvenile-onset T1D (AOO<21) (FIG. 3A) when treated in adulthood, showed uniform reductions in HbA1c over the two-year period as compared to baseline (p=0.015) (FIG. 3A). In contrast, LADA subjects over this two-year observation period did not reduce their HbA1C values (p=0.509) (FIG. 3B). An untreated T1D reference population is also provided for context. This suggests that T1D subjects (vs. LADA) are either exclusively sensitive to BCG vaccination treatment or at least are faster in their blood sugar lowering responses with observations out to two years.

Since there are known differences in BCG strains, the current data from the Tokyo BCG strain were compared to the Sanofi strain-treated Phase I double-blinded clinical trial data, a dataset having similar early ages of onset. The change in HbA1c from the current open-label T1D (n=5) cohort was compared to another Phase 1 T1D cohort (n=3) as well as to a Reference and original placebo groups T1D population (n=57) with the same number of BCG doses (FIG. 3C). The data show similar kinetics and uniformity of all adults with juvenile onset T1D either dosed with the Sanofi or the Tokyo BCG strain. Both strains show a drop in HbA1c levels over 2 years, indicating reproducibility.

Response to Brief 24-Hour BCG Exposure in Monocytes from T1D, LADA, and Non-Diabetic Controls

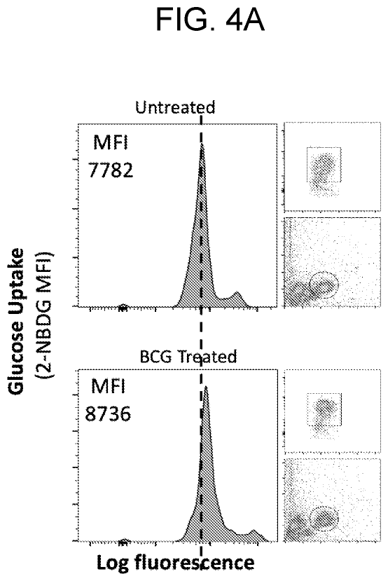

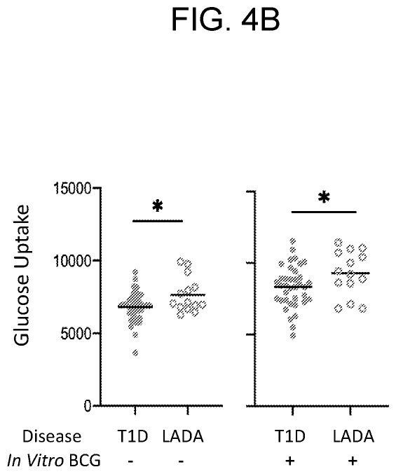

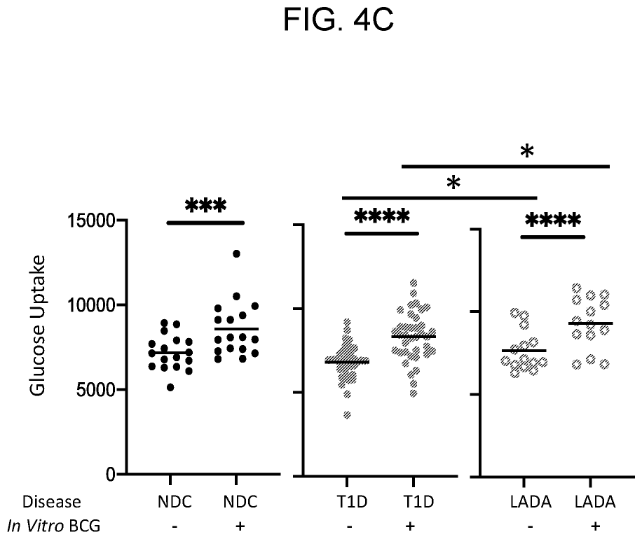

A glucose transport assay, 2-NBDG, was used to corroborate the clinical trial observations (FIG. 4A). Monocytes were isolated from peripheral blood and cultured overnight with or without BCG. The cells were then harvested, washed, counted, incubated with 2-NBDG for 1 hour and labeled with APC-anti-CD14 antibody. The cells were then analyzed using a FASC Canto II flow cytometer. The right shift in the histogram for 2-NBDG fluorescence indicates that the uptake of 2-NBDG (labelled sugar) into the BCG-treated cells is higher than untreated control cells. The outcome is quantified by median fluorescent intensity (MFI).

At baseline, with freshly isolated monocytes, sugar uptake measured by the 2-NBDG assay showed that T1D monocytes had insufficient or depressed sugar transport compared to monocytes from LADA subjects (FIG. 4B, left). The mean fluorescence in T1D monocytes was 6904±170 (n=37) and the mean glucose uptake in LADA monocytes was 7641±325 (n=13) (P<0.0001). With a brief in vitro exposure to BCG, both T1D and LADA monocytes accelerated their sugar transport but T1D monocytes still were less responsive than LADA monocytes (mean 8331±241 versus 9299±421; p<0.0001). These in vitro data indicate that T1D monocytes with in vivo BCG might be more responsive or faster to respond to BCG therapy since they uniquely had the most pronounced deficiencies at baseline.

The in vitro responsiveness to culture with or without BCG was next studied in NDC (n=16), T1D (n=37) and LADA (n=13) monocytes (FIG. 4C). Overall the data show that NDC, T1D and LADA monocytes in culture responded to BCG compared to untreated monocytes and rapidly transported more sugar over a brief one hour monitoring time (P=<0.0001 for each subject group). The results are expressed as the difference in median fluorescence intensity (MFI) between BCG-treated and untreated cells. T1D and LADA diabetic subjects had a difference in baseline sugar transport, with T1D monocytes' sugar transport being sluggish and yielding statistically different results represented as lowered MFI. After BCG treatment in vitro, the LADA monocytes achieved the higher sugar levels inside their cells. Therefore, in T1D with early onset, monocytes have lower basal glucose transport but with BCG exposure, the monocytes have accelerated glucose transport.

Monocyte isolation and culture and the 2-NBDG sugar transport assay were conducted as described in the materials and methods of Example 13.

### Example 3. Glucose Uptake in Bladder Cancer Subjects Who Received BCG Vaccination

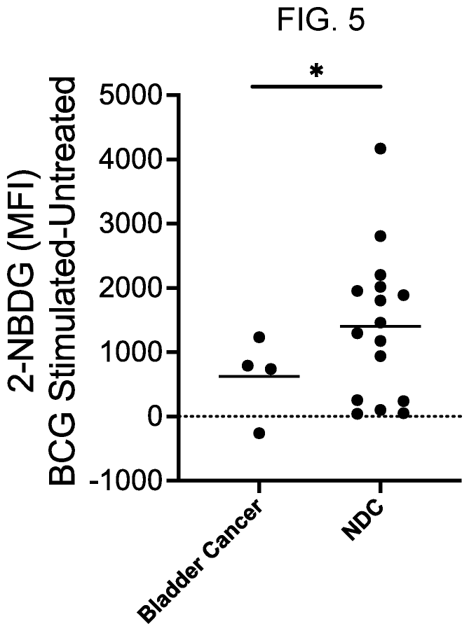

Another clinical setting of BCG exposures are treatments with high dose BCG for bladder cancer. Subjects (who were administered a high dose of BCG in the bladder) were studied months to 20 years after bladder cancer that was treated with BCG (Table 4, FIG. 5). FIG. 5 shows that accelerated glucose transport by monocytes (at baseline and after in vitro BCG exposures) was not observed. All these subjects were US citizens and (based on medical records) received the TICE BCG strain, a strain with less potency than other BCG strains. Furthermore, the stage of bladder cancer may also determine if the BCG from bladder erosions for the cancer will allow systemic population/spread of the administered BCG (this was not feasible data to obtain in this study).

TABLE 4
Subjects vaccinated with BCG for treatment of bladder cancer

| Subject | Birth Country | BCG Strain | Current Age | Dosing Age |
|---|---|---|---|---|
| NDC1 | US | TICE | 71 | 69 |
| NDC1 | US | TICE | 71 | 71 |
| NDC2 | US | TICE | 73 | 71 |
| T2D | US | TICE | 81 | 60 |

These data suggest that not all subjects can exhibit accelerated glucose transport after exposure to BCG, such as, e.g., subjects who have suffered from bladder cancer. Monocyte isolation and culture and the 2-NBDG sugar transport assay were conducted as described in the materials and methods of Example 13.

### Example 4. Response of T2D Monocytes to BCG Exposures In Vitro

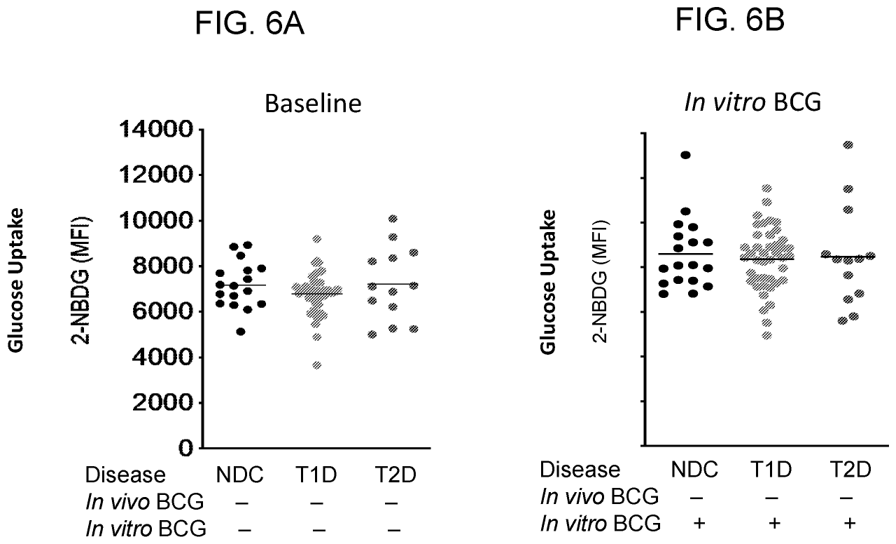

Type 2 diabetes (T2D) was studied using the in vitro glucose uptake assay to possibly generate preliminary data on this form of diabetes being responsive to BCG (FIGS. 6A-6C). The data show that T2D monocytes at baseline have adequate sugar transport and also could respond in vitro to BCG exposures (n=17 NDC, n=42 T1D, n=13 T2D, respectively (FIG. 6A, FIG. 6B)). Further comparisons between NDC and T2D monocytes are also presented (FIG. 6C). There was no difference between NDC and T2D monocytes, either at baseline (untreated panel) or after culture with BCG (BCG-treated panel). These data indicate that type 2 diabetes subjects could benefit from BCG treatment. Monocyte isolation and culture and the 2-NBDG sugar transport assay were conducted as described in the materials and methods of Example 13.

### Example 5. Effect of Metformin on BCG-Induced Glucose Uptake

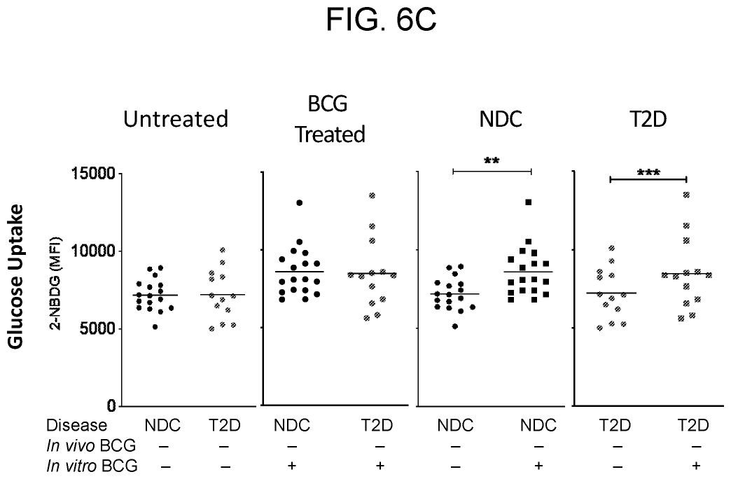

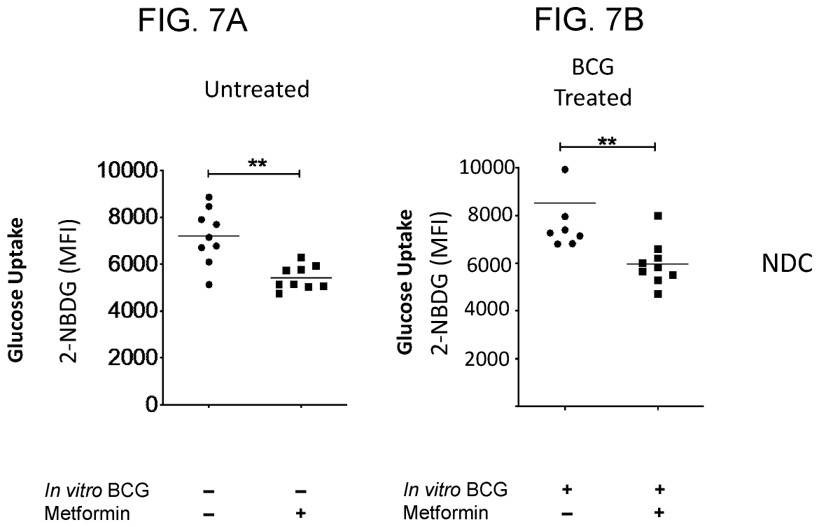

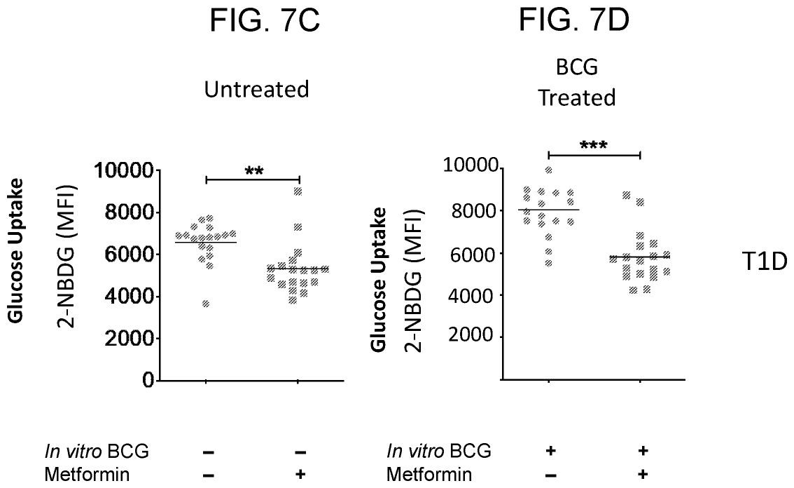

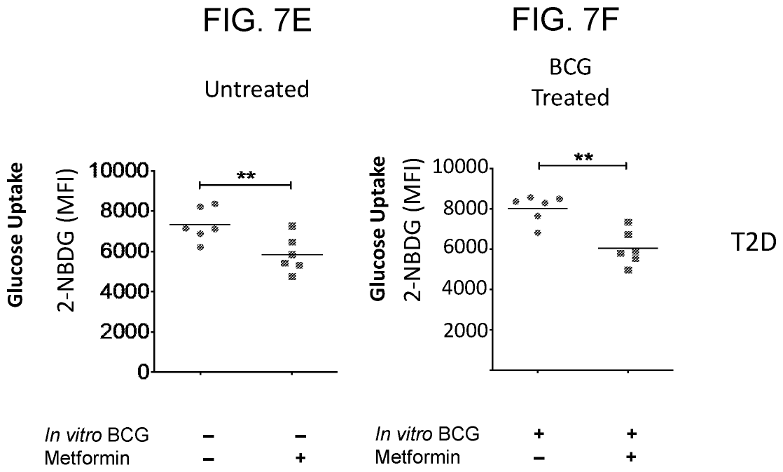

Metformin is taken by many subjects with T2D to help control their blood sugars by increasing insulin sensitivity and by inhibiting glucose neogenesis by the liver. Metformin is also taken by a small fraction of subjects with T1D. It is therefore important to establish whether metformin interferes with BCG with respect to glucose uptake in T1D, as well as T2D in preparation for a potential use of BCG in T2D clinical trials. The effect of metformin and BCG were studied in vitro in human monocytes (FIGS. 7A-F). Previous data suggest that metformin interferes with cytokines induced by BCG through innate immunity as well as with glycolysis pathways in innate and adaptive immunity.

Using the in vitro glucose metabolic assay, 2-NBDG, treatment with metformin in vitro inhibited 2-NBDG uptake in primary human monocytes from all the clinical samples (NDC, T1D, and T2D). Primary monocytes were isolated from NDC, T1D and T2D subjects and were then cultured overnight in the presence or absence of BCG and metformin. The next day the cells were incubated with 2-NBDG and APC-anti-CD14 antibodies and analyzed by flow cytometry. For all forms of diabetes, metformin reduced 2-NBDG uptake by the monocytes, irrespective of whether BCG was present (FIGS. 7A-7F). Thus, in vitro metformin treatment inhibits 2-NBDG uptake in both untreated and BCG treated monocytes and may interfere with the beneficial effects of BCG treatment in vivo for blood sugar control in diabetics. Metformin has its own benefits for blood glucose control in diabetes, its effect likely is not additive with the effect of BCG and it may not be possible to combine both treatments. Monocyte isolation and culture and the 2-NBDG sugar transport assay were conducted as described in the materials and methods of Example 13.

### Example 6. Response to BCG in Murine T1D Model

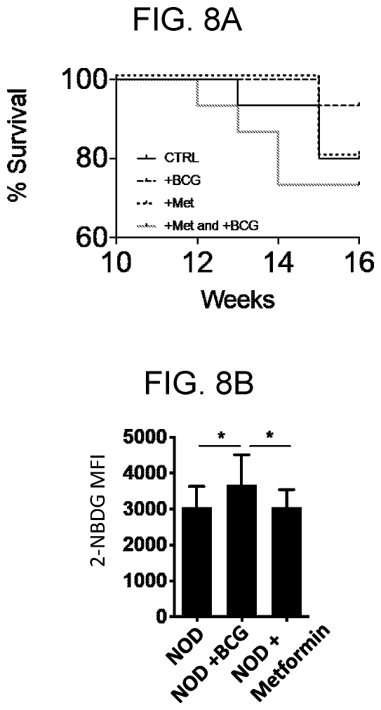

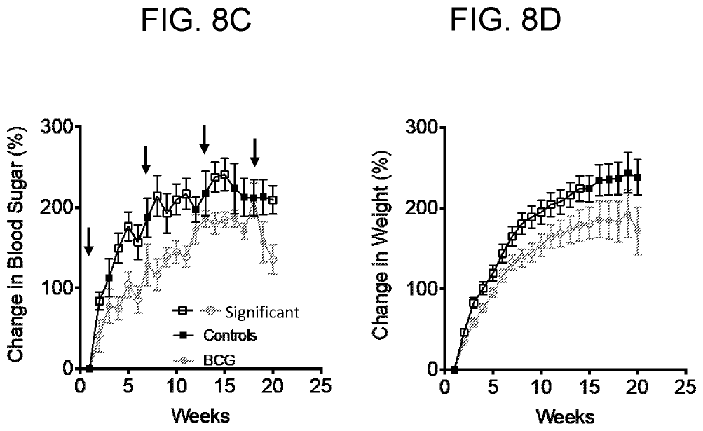

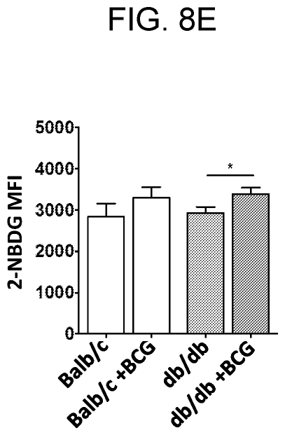

Response to BCG treatment was tested in the non-obese diabetic (NOD) mouse model, a well-established model of T1D. Pre-diabetic (4 weeks old) NOD mice were divided into four groups based on treatment with BCG injections and/or metformin (added to mouse chow). Specifically, the mice were treated with one dose of BCG only; metformin only; both BCG and metformin; or no treatment (FIGS. 8A-B).

The results presented as Kaplan-Meier survival curves for the NOD mice show that BCG increases survival compared to the untreated control group of mice or NOD mice treated with only metformin. In contrast, the combination of BCG and metformin accelerated death in these mice (FIG. 8A). The BCG-only treated mice had the highest survival rate, followed by the untreated controls and the metformin-treated mice. The lowest survival rate was achieved in the group of NOD mice that were treated with both BCG and metformin, again suggesting that BCG and metformin treatments should not be combined. In the mouse, peripheral monocytes cannot be isolated, so instead freshly isolated bone marrow cells from the NOD mice were used to study sugar transport by the 2-NBDG assay (FIG. 8B). BCG significantly increased the 2-NBDG uptake into freshly isolated bone marrow cells from NOD mice (n=11) as compared to untreated NOD mice (n=16, p=0.015) and also as compared to metformin-treated NOD where metformin inhibited the BCG-induced accelerated glycolysis (n=15, p=0.011). These data further support that BCG treatment can increase the rate of glucose uptake in subjects with T1D and that metformin may reduce the efficacy of BCG treatment in subjects. Monocyte isolation and culture, the 2-NBDG sugar transport assay, and NOD mice study were conducted as described in the materials and methods of Example 13.

### Example 7. Response in Murine T2D Model

BCG experiments were performed in the BKS db/db mice, a model of T2D. The mice were divided into 2 groups. One group was treated with multiple BCG injections whereas the other group was untreated. Changes in blood sugars and weight were monitored (FIGS. 8C-D).

Blood sugars for the BCG-treated db/db (n=11) were significantly lower as compared to the untreated db/db mice (n=16). The corresponding change in weight (observed as reduction in weight) in the severely obese mice is shown in FIG. 8D. The open symbols depict timepoints where the difference between CTRL and BCG groups was significant in Student's t testing. Since weight gain and hyperglycemia go hand-in-hand in db/db mice, the lower weight in the BCG-treated mice suggests better health and presumably better glycemic control. Early db/db mouse experiments in which only one single dose of BCG was administered yielded a limited effect. Subsequent studies utilizing administration of four BCG doses yielded robust, clinically meaningful effects.

Bone marrow cells were isolated to test lymphoid sugar transport from all BCG-treated and untreated db/db mice using 2-NBDG uptake experiments (FIG. 8E). For both db/db and control mouse strains the in vivo BCG treatments resulted in bone marrow cells that were capable of increased 2-NBDG uptake. For the db/db mice, the results were significant at p=0.04 (n=10 for both untreated and BCG treated groups). For the BALC/c control mice that also were treated in vivo with BCG under the same conditions, the results trended higher for the BCG-treated group, but non-significantly so (p=0.31; n=4 for both untreated and BCG treated groups). These data indicate that BCG treatment for subjects with T2D could be efficacious. Monocyte isolation and culture, the 2-NBDG sugar transport assay, and NOD mice study were conducted as described in the materials and methods of Example 13.

### Example 8. Determining the Likelihood of a Diabetes Subject to Respond to BCG Therapy on the Basis of Age of Onset of the Diabetic Condition

Using the compositions and methods of the disclosure, a subject (e.g., a human subject) with diabetes (e.g., type 1 diabetes) may be tested for their likelihood to respond to BCG therapy on the basis of the subject's age at the time of onset of the diabetic condition. For example, a physician of skill in the art may ascertain the age that the subject had a the time of onset of the diabetic condition. A finding that the subject had, for example, an age of less than 40 years at the time of onset of the diabetes (such as an age of 39 years, 38 years, 37 years, 36 years, 35 years, 34 years, 33 years, 32 years, 31 years, 30 years, 29 years, 28 years, 27 years, 26 years, 25 years, 24 years, 23 years, 22 years, 21 years, 20 years, 19 years, 18 years, 17 years, 16 years, 15 years, 14 years, 13 years, 12 years, 11 years, 10 years, 9 years, 8 years, 7 years, 6 years, 5 years, 4 years, or 3 years, or less, at the time of onset of the diabetes) may identify the subject as likely to benefit from BCG therapy. The subject may have, for example, a current, chronological age of 50 years or more, such as a chronological age of 51 years, 52 years, 53 years, 54 years, 55 years, 56 years, 57 years, 58 years, 59 years, 60 years, 61 years, 62 years, 63 years, 64 years, or 65 years, or more. Additionally or alternatively, the subject may have been living with the diabetes for an extended period of time, as evidenced, for example, by a finding that the subject has a circulating level of endogenous c-peptide of from 0.1 pM to 5 pM. Regardless of the subject's current chronological age and the duration of the subject's diabetic condition, the subject may be selected for treatment with BCG.

Upon identifying the subject as likely to benefit from BCG treatment, the subject may be administered BCG in one or more doses over the course of a treatment period, such as a treatment period lasting from about one month to about five years. The subject may receive multiple doses of BCG, which may include doses that are evenly spaced apart and/or doses that are unevenly spaced apart.

Following the administration of the final dose of BCG to the subject, the subject may be monitored (e.g., by a physician) to assess the subject's blood glucose and HbA1c levels. Successful treatment with BCG may be indicated, for example, by a determination that, following administration of the BCG to the subject, the subject exhibits a reduction in HbA1c level of about 5% or more, such as a reduction in HbA1C level of from about 5% to about 15%, relative to the HbA1c level prior to treatment. Additional indicators of successful treatment include a finding that the subject exhibits a sustained blood glucose concentration of less than 200 mg/dL.

The subject may be one that, prior to administration of the BCG, is undergoing insulin therapy. Following administration of BCG to the subject, the frequency of the insulin therapy may be reduced. For example, prior to administration of the BCG to the subject, the subject may be receiving insulin in one or more doses per day, week, or month, such as in one or more doses per day, from 2 to 10 doses per week, or from 5 to 25 doses per month. Following administration of the BCG to the subject, the frequency of the insulin therapy may be reduced, for example, such that the subject only receives from 1 to 5 doses of insulin per week or from 2 to 10 doses of insulin per month.

Additionally or alternatively, following administration of the BCG to the subject, the quantity of insulin that is administered to the subject per dose may be reduced. For example, the subject may be receiving insulin an amount of from about 0.1 to about 0.2 units/kg prior to administration of the BCG to the subject. Following administration of the BCG to the subject, the subject may receive insulin in a reduced quantity per dose, such as an amount of from about 0.01 to about 0.05 units/kg.

Alternatively, following administration of the BCG to the subject, the insulin therapy may be ceased altogether.

### Example 9. Determining the Likelihood of a Diabetes Subject to Respond to BCG Therapy on the Basis of Glucose Uptake

Using the compositions and methods of the disclosure, a subject (e.g., a human subject) having diabetes (e.g., type 1 diabetes) may be tested for their likelihood of benefiting from treatment with BCG on the basis of the subject's pre-treatment rate of glucose uptake. For example, prior to administration of BCG, a sample of cells (e.g., peripheral blood cells, such as leukocytes (e.g., monocytes)) may be obtained from the subject and cultured in the presence and absence of BCG. Following the incubation period, the cells may then be exposed to a fluorescent glucose conjugate, such as 2-[N-(7-nitrobenz-2-oxa-1,3-diazol-4-yl)amino]-2-deoxy-D-glucose, among others described herein. Uptake of the glucose conjugate may then be measured by analyzing the fluorescence of the cells at an emission wavelength corresponding to an emission wavelength of the fluorescent glucose conjugate. In this assay, a finding that the rate of glucose uptake exhibited by the cells in the presence of BCG is greater than the rate of glucose uptake exhibited by the cells in the absence of the BCG (or is comparable to the rate of glucose uptake relative to a standard control value) indicates that the subject is likely to respond to BCG treatment.

Upon identifying the subject as likely to benefit from BCG treatment, the subject may be administered BCG in one or more doses over the course of a treatment period, such as a treatment period lasting from about one month to about five years. The subject may receive multiple doses of BCG, which may include doses that are evenly spaced apart and/or doses that are unevenly spaced apart.

Following the administration of the final dose of BCG to the subject, the subject may be monitored by a physician to assess the subject's blood glucose and HbA1c levels. Successful treatment with BCG may be indicated, for example, by a determination that, following administration of the BCG to the subject, the subject exhibits a reduction in HbA1c level of about 5% or more, such as a reduction in HbA1c level of from about 5% to about 15%, relative to the HbA1c level prior to treatment. Additional indicators of successful treatment include a finding that the subject exhibits a sustained blood glucose concentration of less than 200 mg/dL.

The subject may be one that, prior to administration of the BCG, is undergoing insulin therapy. Following administration of BCG to the subject, the frequency of the insulin therapy may be reduced. For example, prior to administration of the BCG to the subject, the subject may be receiving insulin in one or more doses per day, week, or month, such as in one or more doses per day, from 2 to 10 doses per week, or from 5 to 25 doses per month. Following administration of the BCG to the subject, the frequency of the insulin therapy may be reduced, for example, such that the subject only receives from 1 to 5 doses of insulin per week or from 2 to 10 doses of insulin per month.

Additionally or alternatively, following administration of the BCG to the subject, the quantity of insulin that is administered to the subject per dose may be reduced. For example, the subject may be receiving insulin an amount of from about 0.1 to about 0.2 units/kg prior to administration of the BCG to the subject. Following administration of the BCG to the subject, the subject may receive insulin in a reduced quantity per dose, such as an amount of from about 0.01 to about 0.05 units/kg.

Alternatively, following administration of the BCG to the subject, the insulin therapy may be ceased altogether.

### Example 10. Determining the Likelihood of a Diabetes Subject to Respond to BCG Therapy on the Basis of Oxidative Phosphorylation

Using the compositions and methods of the disclosure, a subject (e.g., a human subject) with diabetes (e.g., type 1 diabetes) may be tested for their likelihood of benefiting from treatment with BCG on the basis of the subject's pre-treatment rate of oxidative phosphorylation. For example, prior to administration of BCG, a sample of cells (e.g., peripheral blood cells, such as leukocytes (e.g., monocytes)) may be obtained from the subject and cultured in the presence and absence of BCG. Following the incubation period, the cells may then be analyzed to determine oxygen consumption. In this assay, a finding that the rate of oxygen consumption exhibited by the cells in the presence of BCG is less than the rate of oxygen consumption exhibited by the cells in the absence of the BCG (or is comparable to the rate of oxygen consumption relative to a standard control value) indicates that the subject is likely to respond to BCG treatment.

Upon identifying the subject as likely to benefit from BCG treatment, the subject may be administered BCG in one or more doses over the course of a treatment period, such as a treatment period lasting from about one month to about five years. The subject may receive multiple doses of BCG, which may include doses that are evenly spaced apart and/or doses that are unevenly spaced apart.

Following the administration of the final dose of BCG to the subject, the subject may be monitored by a physician to assess the subject's blood glucose and HbA1c levels. Successful treatment with BCG may be indicated, for example, by a finding that, following administration of the BCG to the subject, the subject exhibits a reduction in HbA1c level of about 5% or more, such as a reduction in HbA1C level of from about 5% to about 15%. Additional indicators of successful treatment include a finding that the subject exhibits a sustained blood glucose concentration of less than 200 mg/dL.

The subject may be one that, prior to administration of the BCG, is undergoing insulin therapy. Following administration of BCG to the subject, the frequency of the insulin therapy may be reduced. For example, prior to administration of the BCG to the subject, the subject may be receiving insulin in one or more doses per day, week, or month, such as in one or more doses per day, from 2 to 10 doses per week, or from 5 to 25 doses per month. Following administration of the BCG to the subject, the frequency of the insulin therapy may be reduced, for example, such that the subject only receives from 1 to 5 doses of insulin per week or from 2 to 10 doses of insulin per month.

Additionally or alternatively, following administration of the BCG to the subject, the quantity of insulin that is administered to the subject per dose may be reduced. For example, the subject may be receiving insulin an amount of from about 0.1 to about 0.2 units/kg prior to administration of the BCG to the subject. Following administration of the BCG to the subject, the subject may receive insulin in a reduced quantity per dose, such as an amount of from about 0.01 to about 0.05 units/kg.

Alternatively, following administration of the BCG to the subject, the insulin therapy may be ceased altogether.

### Example 11. Determining the Likelihood of a Diabetes Subject to Respond to BCG Therapy on the Basis of Aerobic Glycolysis

Using the compositions and methods of the disclosure, a subject (e.g., a human subject) with diabetes (e.g., type 1 diabetes) may be tested for their likelihood of benefiting from treatment with BCG on the basis of the subject's pre-treatment rate of aerobic glycolysis. For example, prior to administration of BCG, a sample of cells (e.g., peripheral blood cells, such as leukocytes (e.g., monocytes)) may be obtained from the subject and cultured in the presence and absence of BCG. Following the incubation period, the cells may then be analyzed to determine the rate of extracellular acidification. In this assay, a finding that the rate of extracellular acidification exhibited by the cells in the presence of BCG is greater than the rate of extracellular acidification exhibited by the cells in the absence of the BCG (or is comparable to the rate of extracellular acidification relative to a standard control value) indicates that the subject is likely to respond to BCG treatment.

Upon identifying the subject as likely to benefit from BCG treatment, the subject may be administered BCG in one or more doses over the course of a treatment period, such as a treatment period lasting from about one month to about five years. The subject may receive multiple doses of BCG, which may include doses that are evenly spaced apart and/or doses that are unevenly spaced apart.

Following the administration of the final dose of BCG to the subject, the subject may be monitored by a physician to assess the subject's blood glucose and HbA1c levels. Successful treatment with BCG may be indicated, for example, by a finding that, following administration of the BCG to the subject, the subject exhibits a reduction in HbA1c level of about 5% or more, such as a reduction in HbA1C level of from about 5% to about 15%. Additional indicators of successful treatment include a finding that the subject exhibits a sustained blood glucose concentration of less than 200 mg/dL.

The subject may be one that, prior to administration of the BCG, is undergoing insulin therapy. Following administration of BCG to the subject, the frequency of the insulin therapy may be reduced. For example, prior to administration of the BCG to the subject, the subject may be receiving insulin in one or more doses per day, week, or month, such as in one or more doses per day, from 2 to 10 doses per week, or from 5 to 25 doses per month. Following administration of the BCG to the subject, the frequency of the insulin therapy may be reduced, for example, such that the subject only receives from 1 to 5 doses of insulin per week or from 2 to 10 doses of insulin per month.

Additionally or alternatively, following administration of the BCG to the subject, the quantity of insulin that is administered to the subject per dose may be reduced. For example, the subject may be receiving insulin an amount of from about 0.1 to about 0.2 units/kg prior to administration of the BCG to the subject. Following administration of the BCG to the subject, the subject may receive insulin in a reduced quantity per dose, such as an amount of from about 0.01 to about 0.05 units/kg.

Alternatively, following administration of the BCG to the subject, the insulin therapy may be ceased altogether.

### Example 12. Determining the Likelihood of a Type 2 Diabetes Subject to Respond to BCG Therapy on the Basis of Age of Onset of the Diabetic Condition

Using the compositions and methods of the disclosure, a subject (e.g., a human subject) with type 2 diabetes may be tested for their likelihood to respond to BCG therapy on the basis of the subject's age at the time of onset of the diabetic condition. For example, a physician of skill in the art may ascertain the age that the subject had a the time of onset of the diabetic condition. A finding that the subject had, for example, an age of less than 40 years at the time of onset of the type 2 diabetes (such as an age of 39 years, 38 years, 37 years, 36 years, 35 years, 34 years, 33 years, 32 years, 31 years, 30 years, 29 years, 28 years, 27 years, 26 years, 25 years, 24 years, 23 years, 22 years, 21 years, 20 years, 19 years, 18 years, 17 years, 16 years, 15 years, 14 years, 13 years, 12 years, 11 years, 10 years, 9 years, 8 years, 7 years, 6 years, 5 years, 4 years, or 3 years, or less, at the time of onset of the diabetes) may identify the subject as likely to benefit from BCG therapy. The subject may have, for example, a current, chronological age of 50 years or more, such as a chronological age of 51 years, 52 years, 53 years, 54 years, 55 years, 56 years, 57 years, 58 years, 59 years, 60 years, 61 years, 62 years, 63 years, 64 years, or 65 years, or more. Additionally or alternatively, the subject may have been living with the type 2 diabetes for an extended period of time. Regardless of the subject's current chronological age and the duration of the subject's diabetic condition, the subject may be selected for treatment with BCG.

Additionally or alternatively, other assays, such as those described in Examples 3-5, above, can be used to identify the subject's likelihood of benefiting from BCG treatment.

Upon identifying the subject as likely to benefit from BCG treatment, the subject may be administered BCG in one or more doses over the course of a treatment period, such as a treatment period lasting from about one month to about five years. The subject may receive multiple doses of BCG, which may include doses that are evenly spaced apart and/or doses that are unevenly spaced apart.

Following the administration of the final dose of BCG to the subject, the subject may be monitored (e.g., by a physician) to assess the subject's blood glucose and HbA1c levels. Successful treatment with BCG may be indicated, for example, by a determination that, following administration of the BCG to the subject, the subject exhibits a reduction in HbA1c level of about 5% or more, such as a reduction in HbA1C level of from about 5% to about 15%, relative to the HbA1c level prior to treatment. Additional indicators of successful treatment include a finding that the subject exhibits a sustained blood glucose concentration of less than 200 mg/dL.

### Example 13. Materials and Methods

Monocyte Isolation and Culture

Whole blood samples were collected from human subjects and monocytes were isolated using the StemCell Technologies Direct Human Monocyte Isolation kit (Stemcell Technologies, Vancouver, BC, Canada) and cultured overnight at 37° C. and 5% CO₂. The culture media was Immunocult media (Stemcell Technologies) the cells were placed into 24-well Nunc UpCell culture plates (Thermofisher Scientific, Waltham, Mass.) at 1×10⁶ monocytes per well. Culture conditions included with or without BCG (1×10⁶ CFU/well, BCG Japan, Tokyo, Japan) and with or without metformin (1 mM, Sigma Aldrich, St. Louis, Mo.). UpCell plates have temperature-responsive surfaces that allow cells to attach at 37° C., but release the cells at room temperature or below. To harvest the cells, the warm culture media was replaced with 1 mL of cold XFP buffer (Agilent Technologies, Santa Clara, Calif.) and the plates left at room temperature for 20 min to allow the cells to detach. The cells were then harvested and transferred to Eppendorf tubes.

2-NBDG Sugar Transport Assay

The 2-NBDG sugar uptake assay was devised to quantify the rate of glucose uptake in vitro and to correlate glucose uptake with response to BCG therapy. Monocytes were isolated from subjects (e.g., T1D, LADA, NDC, and T2D subjects) and cultured for 24 hours on Nunc UpCell 24-Well plates (Thermo Scientific, Cat #174899) at a concentration of 1×10⁶ cells/mL in 1 mL of Immunocult-SF Media (Stemcell Technologies), with or without 1×10⁵ CFU BCG added to the media (Multiplicity of Infection, MOI 0.1). UpCell plates have a coating that exhibits a temperature dependent transition from hydrophobic to hydrophilic. Thus, the coating allows cell attachment at 37° C., whereas the cells will detach at room temperature or lower. Cells were recovered, washed, and incubated for 30 minutes at 37° C. with 100 uM 2-NBDG ((2-(N-(7-Nitrobenz-2-oxa-1,3-diazol-4-yl)Amino)-2-Deoxyglucose); ThermoFisher Cat #N13195) in Hepes buffered RPMI without glucose (XFp medium, pH 7.4; Agilent Technologies, Wilmington, Del., USA. Cat #103576-100). The cells were then washed with XFp medium and analyzed on a BD FacsCanto II flow cytometer. Data was processed using FlowJo software to determine 2-NBDG median fluorescence intensity (MFI).

For the direct measurements of aerobic glycolysis, changes in Oxygen Consumption Rate (OCR), a measure of oxidative phosphorylation, and Extracellular Acidification Rate (ECAR), a measure of aerobic glycolysis, were measured using a Seahorse XFp Analyzer (Agilent). Isolated monocytes were cultured (2×10⁵ per well) for seven days with or without 1×10⁶ CFU of BCG (MOI=5) in Immunocult-SF Media (Stemcell Technologies). The cells were then washed with SEAHORSE™ XFp medium and analyzed using the SEAHORSE™ XFp Analyzer. After obtaining three baseline data points, Rotenone and AntiMycin A (final concentration, 0.5 uM) were added to the media to inhibit oxygen usage due to oxidative phosphorylation.

Animal Studies

Five-week-old BALB/c male, NOD female, and db/db male mice (The Jackson Laboratory, Bar Harbor, Me., USA) were housed under specific pathogen-free conditions. The mice were carefully monitored daily by the staff who completed the course in animal care, freely fed on normal diet and were not fasted before a challenge or assessment. When the mice showed signs of cachexia such as prominent loss of body weight or became moribund, they were immediately euthanized.

The mice were housed in five animals per cage (four animals per cage after they reached a weight of >25 grams). Body weight and blood sugar measurements were taken weekly to monitor diabetes progression. Blood was obtained from tail nicks using a 5.5 mm animal lancet (MEDipoint; Mineola, N.Y.) and glucose was measured using an Aviva Plus glucometer (Accuchek; Indianapolis, Ind.). BCG was injected by footpad at a dose of 25 μL of BCG (2 mg/ml in saline; Japan BCG Laboratory; Tokyo, Japan). Treated and untreated mice were sacrificed using CO₂ and bone marrow was isolated from femurs and purified with RBC Lysis buffer (Thermofisher Scientific, Waltham, Mass.). One million cells were resuspended in 50 μL Agilent Seahorse XFp media and mixed with 50 μL of 2-NBDG (0.2 mM in XFp, (Agilent, Santa Clara, Calif., USA). The suspension was incubated in a CO₂-free incubator at 37° C. for 30 minutes, washed and resuspended in 300 μL XFP. The cells were analyzed on a FacsCanto II flow cytometer. Data was analyzed using FlowJo software.

NOD mice (Jackson Labs; Bar Harbor, Me.) were used as a models for T1D. A subgroup of NOD mice were started on metformin (0.1%) mixed into mouse chow (ScottPharma Solutions; Marlborough, Mass.) at 6 weeks of age. After one month, half of the mice in the metformin group, and half of the mice in the control group were injected a single time with BCG. BALB/c and BKS db/db mice (Jackson Labs; Bar Harbor, Me.) were used as models for non-diabetic control and T2D, respectively. Early experiments with the db/db mouse revealed a single injection of BCG at 7 weeks of age was insufficient to change blood sugar or weight, four BCG injections in alternating rear footpads were administered when the mice were 8, 14, 20 and 23 weeks old (data shown). BCG was at a dose of 25 μL of BCG (2 mg/ml in saline; Japan BCG Laboratory; Tokyo, Japan).

### Other Embodiments

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations described herein following, in general, the principles described herein and including such departures from the invention that come within known or customary practice within the art to which the invention pertains and may be applied to the essential features hereinbefore set forth, and follows in the scope of the claims.

Other embodiments are within the claims.

1. A method of treating diabetes in a subject in need thereof comprising administering a therapeutically effective amount of Bacillus Calmette-Guerin (BCG) to the subject, wherein, prior to administration of the BCG to the subject, the subject has been determined to:
a) have had an age of less than 40 years at the onset of the diabetes;
b) exhibit a rate of glucose uptake that is less than a reference rate of glucose uptake;
c) exhibit a rate of oxidative phosphorylation that is greater than a reference rate of oxidative phosphorylation; and/or
d) exhibit a rate of aerobic glycolysis that is less than a reference rate of aerobic glycolysis.

2. A method of selecting a subject with diabetes for treatment with BCG comprising:
a) determining an age of onset of the diabetes in the subject, wherein a determination that the subject was less than 40 years old at the onset of the diabetes identifies the subject as likely to respond to the BCG;
b) determining a rate of glucose uptake exhibited by the subject, wherein a determination that the subject exhibits a rate of glucose uptake that is less than a reference rate of glucose uptake identifies the subject as likely to respond to the BCG, optionally wherein the reference rate of glucose uptake is about 6,000 mean fluorescence intensity (MFI) units to about 10,000 MFI units, as assessed by subjecting a cell of the subject to a fluorescently-labeled glucose uptake assay;
c) determining a rate of oxidative phosphorylation exhibited by the subject, wherein a determination that the subject exhibits a rate of oxidative phosphorylation that is greater than a reference rate of oxidative phosphorylation identifies the subject as likely to respond to the BCG, optionally wherein the reference rate of oxidative phosphorylation is from about 25 pmol of O₂ consumption per minute to about 150 pmol of O₂ consumption per minute; and/or
d) determining a rate of aerobic glycolysis exhibited by the subject, wherein a determination that the subject exhibits a rate of aerobic glycolysis that is less than a reference rate of aerobic glycolysis identifies the subject as likely to respond to the BCG, optionally wherein the reference rate of aerobic glycolysis is from about 5 to about 50 mpH units per minute,
optionally wherein the method further comprises administering a therapeutically effective amount of BCG to the subject identified as likely to respond to the BCG.

3. A method of treating diabetes in a subject in need thereof comprising administering a therapeutically effective amount of BCG to the subject, wherein the subject has been selected for treatment with BCG by a method comprising:
a) providing first and second samples of cells obtained from the subject prior to administration of the BCG to the subject;
b) culturing the first sample of cells in the absence of BCG and the second sample of cells in the presence of BCG; and
c) evaluating a rate of glucose uptake in the first and second samples of cells,
wherein the rate of glucose uptake in the second sample of cells is determined to be greater than the rate of glucose uptake in the first sample of cells.

4. A method of selecting a subject with diabetes for treatment with BCG comprising:
a) providing first and second samples of cells obtained from the subject prior to administration of BCG to the subject;
b) culturing the first sample of cells in the absence of BCG and the second sample of cells in the presence of BCG; and
c) evaluating a rate of glucose uptake in the first and second samples of cells,
wherein a determination that the rate of glucose uptake in the second sample of cells is greater than the rate of glucose uptake in the first sample of cells identifies the subject as likely to respond to the BCG, optionally wherein the method further comprises administering a therapeutically effective amount of BCG to the subject identified as likely to respond to the BCG.

5. A method of treating diabetes in a subject in need thereof comprising administering a therapeutically effective amount of BCG to the subject, wherein the subject has been selected for treatment with BCG by a method comprising:
a) providing first and second samples of cells obtained from the subject prior to administration of the BCG to the subject;
b) culturing the first sample of cells in the absence of BCG and the second sample of cells in the presence of BCG; and
c) evaluating a rate of oxidative phosphorylation in the first and second samples of cells,
wherein the rate of oxidative phosphorylation in the second sample of cells is determined to be less than the rate of oxidative phosphorylation in the first sample of cells.

6. A method of selecting a subject with diabetes for treatment with BCG comprising:
a) providing first and second samples of cells obtained from the subject prior to administration of the BCG to the subject;
b) culturing the first sample of cells in the absence of BCG and the second sample of cells in the presence of BCG; and
c) evaluating a rate of oxidative phosphorylation in the first and second samples of cells,
wherein a determination that the rate of oxidative phosphorylation in the second sample of cells is less than the rate of oxidative phosphorylation in the first sample of cells identifies the subject as likely to respond to the BCG, optionally wherein the method further comprises administering a therapeutically effective amount of BCG to the subject identified as likely to respond to the BCG.

7. A method of treating diabetes in a subject in need thereof comprising administering a therapeutically effective amount of BCG to the subject, wherein the subject has been selected for treatment with BCG by a method comprising:
a) providing first and second samples of cells obtained from the subject prior to administration of the BCG to the subject;
b) culturing the first sample of cells in the absence of BCG and the second sample of cells in the presence of BCG; and
c) evaluating a rate of aerobic glycolysis in the first and second samples of cells,
wherein the rate of aerobic glycolysis in the second sample of cells is determined to be greater than the rate of aerobic glycolysis in the first sample of cells.

8. A method of selecting a subject with diabetes for treatment with BCG comprising:
a) providing first and second samples of cells obtained from the subject prior to administration of the BCG to the subject;
b) culturing the first sample of cells in the absence of BCG and the second sample of cells in the presence of BCG; and
c) evaluating the rate of aerobic glycolysis in the first and second samples of cells,
wherein a determination that the rate of aerobic glycolysis in the second sample of cells is greater than the rate of aerobic glycolysis in the first sample of cells identifies the subject as likely to respond to the BCG, optionally wherein the method further comprises administering a therapeutically effective amount of BCG to the subject identified as likely to respond to the BCG.

9. The method of any one of claims 1-8, wherein the subject is or was determined to be 37 years old or younger at the onset of the diabetes.

10. The method of claim 9, wherein the subject is or was determined to be 35 years old or younger at the onset of the diabetes.

11. The method of claim 10, wherein the subject is or was determined to be 33 years old or younger at the onset of the diabetes.

12. The method of claim 11, wherein the subject is or was determined to be 31 years old or younger at the onset of the diabetes.

13. The method of claim 12, wherein the subject is or was determined to be 29 years old or younger at the onset of the diabetes.

14. The method of claim 13, wherein the subject is or was determined to be 27 years old or younger at the onset of the diabetes.

15. The method of claim 14, wherein the subject is or was determined to be 25 years old or younger at the onset of the diabetes.

16. The method of claim 15, wherein the subject is or was determined to be 23 years old or younger at the onset of the diabetes.

17. The method of claim 16, wherein the subject is or was determined to be 21 years old or younger at the onset of the diabetes.

18. The method of claim 17, wherein the subject is or was determined to be 18 years old or younger at the onset of the diabetes.

19. The method of claim 18, wherein the subject is or was determined to be 15 years old or younger at the onset of the diabetes.

20. The method of claim 19, wherein the subject is or was determined to be 14 years old or younger at the onset of the diabetes.

21. The method of claim 20, wherein the subject is or was determined to be 13 years old or younger at the onset of the diabetes.

22. The method of claim 21, wherein the subject is or was determined to be 12 years old or younger at the onset of the diabetes.

23. The method of claim 22, wherein the subject is or was determined to be 11 years old or younger at the onset of the diabetes.

24. The method of claim 23, wherein the subject is or was determined to be 10 years old or younger at the onset of the diabetes.

25. The method of claim 24, wherein the subject is or was determined to be 9 years old or younger at the onset of the diabetes.

26. The method of any one of claims 1-8, wherein the subject is or was determined to be about 3 years to about 35 years old at the onset of the diabetes.

27. The method of claim 26, wherein the subject is or was determined to be about 3 years to about 33 years old at the onset of the diabetes.

28. The method of claim 27, wherein the subject is or was determined to be about 3 years to about 31 years old at the onset of the diabetes.

29. The method of claim 28, wherein the subject is or was determined to be about 3 years to about 29 years old at the onset of the diabetes.

30. The method of claim 29, wherein the subject is or was determined to be about 3 years to about 27 years old at the onset of the diabetes.

31. The method of claim 30, wherein the subject is or was determined to be about 3 years to about 25 years old at the onset of the diabetes.

32. The method of claim 31, wherein the subject is or was determined to be about 3 years to about 23 years old at the onset of the diabetes.

33. The method of claim 32, wherein the subject is or was determined to be about 3 years to about 21 years old at the onset of the diabetes.

34. The method of claim 33, wherein the subject is or was determined to be about 3 years to about 18 years old at the onset of the diabetes.

35. The method of claim 34, wherein the subject is or was determined to be about 3 years to about 15 years old at the onset of the diabetes.

36. The method of claim 35, wherein the subject is or was determined to be about 3 years to about 14 years old at the onset of the diabetes.

37. The method of claim 36, wherein the subject is or was determined to be about 3 years to about 13 years old at the onset of the diabetes.

38. The method of claim 37, wherein the subject is or was determined to be about 3 years to about 12 years old at the onset of the diabetes.

39. The method of claim 38, wherein the subject is or was determined to be about 3 years to about 11 years old at the onset of the diabetes.

40. The method of claim 39, wherein the subject is or was determined to be about 3 years to about 10 years old at the onset of the diabetes.

41. The method of claim 40, wherein the subject is or was determined to be about 3 years to about 9 years old at the onset of the diabetes.

42. The method of any one of claims 1-41, wherein the subject first presented one or more symptoms of the diabetes at an age of from about 3 years to about 21 years.

43. The method of claim 42, wherein the subject first presented one or more symptoms of the diabetes at an age of from about 3 years to about 18 years.

44. The method of claim 43, wherein the subject first presented one or more symptoms of the diabetes at an age of from about 3 years to about 15 years.

45. The method of claim 44, wherein the subject first presented one or more symptoms of the diabetes at an age of from about 3 years to about 14 years.

46. The method of claim 45, wherein the subject first presented one or more symptoms of the diabetes at an age of from about 3 years to about 13 years.

47. The method of claim 46, wherein the subject first presented one or more symptoms of the diabetes at an age of from about 3 years to about 12 years.

48. The method of claim 47, wherein the subject first presented one or more symptoms of the diabetes at an age of from about 3 years to about 11 years.

49. The method of claim 48, wherein the subject first presented one or more symptoms of the diabetes at an age of from about 3 years to about 10 years.

50. The method of claim 49, wherein the subject first presented one or more symptoms of the diabetes at an age of from about 3 years to about 9 years.

51. The method of any one of claims 42-50, wherein the one or more symptoms comprise:
a) a blood glucose concentration of about 200 mg/dL or greater;
b) a glycated hemoglobin (HbA1c) level of 6.5% or greater;
c) a blood glucose concentration of about 100 mg/dL or greater in the subject in a fasting state, optionally wherein the fasting state is one in which the subject has not eaten for the previous 8-12 hours relative to the time at which the blood glucose concentration is measured; and/or
d) a presence of anti-glutamic acid decarboxylase (GAD) autoantibodies in peripheral blood isolated from the subject.

52. The method of claim 51, wherein the one or more symptoms comprise a blood glucose concentration of from about 100 mg/dL to about 125 mg/dL in the subject in the fasting state.

53. The method of claim 52, wherein the one or more symptoms comprise a blood glucose concentration of about 126 mg/dL or greater in the subject in the fasting state.

54. The method of any one of claims 1-53, wherein the subject was from about 3 years to about 21 years old at the time of diagnosis of the diabetes in the subject.

55. The method of claim 54, wherein the subject was from about 3 years to about 18 years old at the time of diagnosis of the diabetes in the subject.

56. The method of claim 55, wherein the subject was from about 3 years to about 15 years old at the time of diagnosis of the diabetes in the subject.

57. The method of claim 56, wherein the subject was from about 3 years to about 14 years old at the time of diagnosis of the diabetes in the subject.

58. The method of claim 57, wherein the subject was from about 3 years to about 13 years old at the time of diagnosis of the diabetes in the subject.

59. The method of claim 58, wherein the subject was from about 3 years to about 12 years old at the time of diagnosis of the diabetes in the subject.

60. The method of claim 59, wherein the subject was from about 3 years to about 11 years old at the time of diagnosis of the diabetes in the subject.

61. The method of claim 60, wherein the subject was from about 3 years to about 10 years old at the time of diagnosis of the diabetes in the subject.

62. The method of claim 61, wherein the subject was from about 3 years to about 9 years old at the time of diagnosis of the diabetes in the subject.

63. The method of any one of claims 1-62, wherein the subject has been diagnosed as having juvenile onset diabetes.

64. The method of any one of claims 1-63, wherein, prior to administration of the BCG to the subject, the subject has been determined to exhibit a rate of glucose uptake that is less than a reference rate of glucose uptake.

65. The method of any one of claims 1-64, wherein, prior to administration of the BCG to the subject, the subject has been determined to exhibit a rate of oxidative phosphorylation that is greater than a reference rate of oxidative phosphorylation.

66. The method of any one of claims 1-65, wherein, prior to administration of the BCG to the subject, the subject has been determined to exhibit a rate of aerobic glycolysis that is less than a reference rate of aerobic glycolysis.

67. The method of any one of claims 1, 2, and 9-66, wherein the rate of glucose uptake exhibited by the subject is assessed by evaluating the rate of glucose uptake in a sample of cells obtained from the subject.

68. The method of claim 67, wherein the cells obtained from the subject comprise peripheral blood cells.

69. The method of claim 68, wherein the cells obtained from the subject comprise leukocytes.

70. The method of claim 69, wherein the cells obtained from the subject comprise monocytes.

71. The method of any one of claims 67-70, wherein the rate of glucose uptake in the sample of cells obtained from the subject is evaluated by a method comprising:
a) contacting the cells with a compound comprising glucose covalently bound to a fluorophore;
b) washing the cells to remove any such compound that has not been internalized by the cells; and
c) measuring the fluorescence of the cells at a wavelength corresponding to an emission wavelength of the fluorophore,
wherein intensity of fluorescence exhibited by the cells at the emission wavelength of the fluorophore correlates with uptake of glucose by the cells.

72. The method of claim 71, wherein the fluorophore comprises a moiety selected from 7-nitrobenz-2-oxa-1,3-diazol-4-yl (NBD), methoxycoumarin, dialkylaminocoumarin, hydroxycoumarin, aminomethylcoumarin acetate (AMCA), 6,8-difluoro-7-hydroxy-3-carboxycoumarin (PACIFIC BLUE™ dye), 5-(dimethylamino)naphthalene-1-sulfonyl (Dansyl), pyrene, 7-amino-3-{[(2,5-dioxopyrrolidin-1-yl)oxy]-2-oxoethyl}-4-methyl-2-oxo-2H-chromene-6-sulfonic acid (ALEXA FLUOR 350™), 6,8-difluoro-7-hydroxy-4-methylcoumarin (MARINA BLUE™ dye), N-(2-aminoethyl)-4-{5-[4-(dimethylamino)phenyl]-1,3-oxazol-2-yl}benzenesulfonamide (DAPOXYL™ dye), 2,3,5,6-Tetramethyl-1H,7H-pyrazolo[1,2-a]pyrazole-1,7-dione (Bimane), 4-{[4-(Diethylamino)phenyl][4-(ethylamino)naphthalen-2-yl]methylidene}-N,N-diethylcyclohexa-2,5-dien-1-iminium (CASCADE BLUE™ dye), tris(N,N-diethylethanaminium) 8-[2-(4-{[(2,5-dioxopyrrolidin-1-yl)oxy]carbonyl}piperidin-1-yl)-2-oxoethoxy]pyrene-1,3,6-trisulfonate (ALEXA FLUOR 405™), N,N-diethylethanaminium [9-{6-[(2,5-dioxopyrrolidin-1-yl)oxy]-6-oxohexyl}-8,8-dimethyl-2-oxo-4-(trifluoromethyl)-8,9-dihydro-2H-benzo[g]chromen-6-yl]methanesulfonate (ALEXA FLUOR 430™) 1-[({4-[(7-nitro-2,1,3-benzoxadiazol-4-yl)amino]phenyl}acetyl)oxy]pyrrolidine-2,5-dione (QSY™ dye), fluorescein, 2-(6-amino-3-iminio-4,5-disulfonato-3H-xanthen-9-yl)-5-((2,3,5,6-tetrafluorophenoxy)carbonyl)benzoate (ALEXA FLUOR 488™), 2′,7′-Difluoro-3′,6′-dihydroxy-3H-spiro[isobenzofuran-1,9′-xanthen]-3-one (OREGON GREEN™ 488), 1,3,5,7,8-pentamethyl-4,4-difluorro-4-bora-3a,4a-diaza-s-indacene (BODIPY™493/503), rhodamine green, and rhodamine red.

73. The method of claim 72, wherein the compound is 2-[N-(7-nitrobenz-2-oxa-1,3-diazol-4-yl)amino]-2-deoxy-D-glucose.

74. The method of any one of claims 1, 2, and 9-73, wherein the reference rate of glucose uptake is a rate of glucose uptake exhibited by a reference subject that does not have diabetes.

75. The method of claim 74, wherein the reference rate of glucose uptake is assessed by evaluating the rate of glucose uptake in a sample of cells obtained from the reference subject.

76. The method of claim 75, wherein the cells obtained from the reference subject comprise peripheral blood cells.

77. The method of claim 76, wherein the cells obtained from the reference subject comprise leukocytes.

78. The method of claim 77, wherein the cells obtained from the reference subject comprise monocytes.

79. The method of any one of claims 75-78, wherein the rate of glucose uptake in the sample of cells obtained from the reference subject is evaluated by a method comprising:
a) contacting the cells with a compound comprising glucose covalently bound to a fluorophore;
b) washing the cells to remove any such compound that has not been internalized by the cells; and
c) measuring the fluorescence of the cells at a wavelength corresponding to an emission wavelength of the fluorophore,
wherein intensity of fluorescence exhibited by the cells at the emission wavelength of the fluorophore correlates with uptake of glucose by the cells.

80. The method of claim 79, wherein the fluorophore comprises a moiety selected from 7-nitrobenz-2-oxa-1,3-diazol-4-yl (NBD), methoxycoumarin, dialkylaminocoumarin, hydroxycoumarin, aminomethylcoumarin acetate (AMCA), 6,8-difluoro-7-hydroxy-3-carboxycoumarin (PACIFIC BLUE™ dye), 5-(dimethylamino)naphthalene-1-sulfonyl (Dansyl), pyrene, 7-amino-3-{[(2,5-dioxopyrrolidin-1-yl)oxy]-2-oxoethyl}-4-methyl-2-oxo-2H-chromene-6-sulfonic acid (ALEXA FLUOR 350™), 6,8-difluoro-7-hydroxy-4-methylcoumarin (MARINA BLUE™ dye), N-(2-aminoethyl)-4-{5-[4-(dimethylamino)phenyl]-1,3-oxazol-2-yl}benzenesulfonamide (DAPOXYL™ dye), 2,3,5,6-Tetramethyl-1H,7H-pyrazolo[1,2-a]pyrazole-1,7-dione (Bimane), 4-{[4-(Diethylamino)phenyl][4-(ethylamino)naphthalen-2-yl]methylidene}-N,N-diethylcyclohexa-2,5-dien-1-iminium (CASCADE BLUE™ dye), tris(N,N-diethylethanaminium) 8-[2-(4-{[(2,5-dioxopyrrolidin-1-yl)oxy]carbonyl}piperidin-1-yl)-2-oxoethoxy]pyrene-1,3,6-trisulfonate (ALEXA FLUOR 405™), N,N-diethylethanaminium [9-{6-[(2,5-dioxopyrrolidin-1-yl)oxy]-6-oxohexyl}-8,8-dimethyl-2-oxo-4-(trifluoromethyl)-8,9-dihydro-2H-benzo[g]chromen-6-yl]methanesulfonate (ALEXA FLUOR 430™) 1-[({4-[(7-nitro-2,1,3-benzoxadiazol-4-yl)amino]phenyl}acetyl)oxy]pyrrolidine-2,5-dione (QSY™ dye), fluorescein, 2-(6-amino-3-iminio-4,5-disulfonato-3H-xanthen-9-yl)-5-((2,3,5,6-tetrafluorophenoxy)carbonyl)benzoate (ALEXA FLUOR 488™), 2′,7′-Difluoro-3′,6′-dihydroxy-3H-spiro[isobenzofuran-1,9′-xanthen]-3-one (OREGON GREEN™ 488), 1,3,5,7,8-pentamethyl-4,4-difluorro-4-bora-3a,4a-diaza-s-indacene (BODIPY™493/503), rhodamine green, and rhodamine red.

81. The method of claim 80, wherein the compound is 2-[N-(7-nitrobenz-2-oxa-1,3-diazol-4-yl)amino]-2-deoxy-D-glucose.

82. The method of any one of claims 1, 2, and 9-81, wherein the rate of oxidative phosphorylation exhibited by the subject is assessed by evaluating the rate of oxygen consumption in a sample of cells obtained from the subject.

83. The method of any one of claims 1, 2, and 9-82, wherein the rate of aerobic glycolysis exhibited by the subject is assessed by evaluating the rate of extracellular acidification in a sample of cells obtained from the subject.

84. The method of claim 82 or 83, wherein the cells obtained from the subject comprise peripheral blood cells.

85. The method of claim 84, wherein the cells obtained from the subject comprise leukocytes.

86. The method of claim 85, wherein the cells obtained from the subject comprise monocytes.

87. The method of any one of claims 1, 2, and 9-86, wherein the reference rate of oxidative phosphorylation is a rate of oxidative phosphorylation exhibited by a reference subject that does not have diabetes.

88. The method of claim 87, wherein the reference rate of oxidative phosphorylation is assessed by evaluating the rate of oxygen consumption in a sample of cells obtained from the reference subject.

89. The method of any one of claims 1, 2, and 9-88, wherein the reference rate of aerobic glycolysis is a rate of aerobic glycolysis exhibited by a reference subject that does not have diabetes.

90. The method of claim 89, wherein the reference rate of aerobic glycolysis is assessed by evaluating the rate of extracellular acidification in a sample of cells obtained from the reference subject.

91. The method of claim 89 or 90, wherein the cells obtained from the reference subject comprise peripheral blood cells.

92. The method of claim 91, wherein the cells obtained from the reference subject comprise leukocytes.

93. The method of claim 92, wherein the cells obtained from the reference subject comprise monocytes.

94. The method of any one of claims 3-93, wherein the first and/or second sample of cells obtained from the subject prior to administration of BCG comprise peripheral blood cells.

95. The method of claim 94, wherein the first and/or second sample of cells obtained from the subject prior to administration of BCG comprise leukocytes.

96. The method of claim 95, wherein the first and/or second sample of cells obtained from the subject prior to administration of BCG comprise monocytes.

97. The method of any one of claims 3-96, wherein the second sample of cells is cultured with BCG for from about 1 hour to about 1 week.

98. The method of claim 97, wherein the second sample of cells is cultured with the BCG for from about 12 hours to about 48 hours.

99. The method of claim 98, wherein the second sample of cells is cultured with the BCG for from about 18 hours to about 36 hours.

100. The method of claim 99, wherein the second sample of cells is cultured with the BCG for about 24 hours.

101. The method of any one of claims 3, 4, and 9-100, wherein the rate of glucose uptake in the first and second samples of cells obtained from the subject prior to administration of BCG is evaluated by a method comprising:
a) contacting the first and second samples of cells with a compound comprising glucose covalently bound to a fluorophore;
b) washing the first and second samples of cells to remove any such compound that has not been internalized by the cells; and
c) measuring the fluorescence of the first and second samples of cells at a wavelength corresponding to an emission wavelength of the fluorophore,
wherein intensity of fluorescence exhibited by the first and second samples of cells at the emission wavelength of the fluorophore correlates with uptake of glucose by the first and second samples of cells.

102. The method of claim 101, wherein the fluorophore comprises a moiety selected from 7-nitrobenz-2-oxa-1,3-diazol-4-yl (NBD), methoxycoumarin, dialkylaminocoumarin, hydroxycoumarin, aminomethylcoumarin acetate (AMCA), 6,8-difluoro-7-hydroxy-3-carboxycoumarin (PACIFIC BLUE™ dye), 5-(dimethylamino)naphthalene-1-sulfonyl (Dansyl), pyrene, 7-amino-3-{[(2,5-dioxopyrrolidin-1-yl)oxy]-2-oxoethyl}-4-methyl-2-oxo-2H-chromene-6-sulfonic acid (ALEXA FLUOR 350™), 6,8-difluoro-7-hydroxy-4-methylcoumarin (MARINA BLUE™ dye), N-(2-aminoethyl)-4-{5-[4-(dimethylamino)phenyl]-1,3-oxazol-2-yl}benzenesulfonamide (DAPOXYL™ dye), 2,3,5,6-Tetramethyl-1H,7H-pyrazolo[1,2-a]pyrazole-1,7-dione (Bimane), 4-{[4-(Diethylamino)phenyl][4-(ethylamino)naphthalen-2-yl]methylidene}-N,N-diethylcyclohexa-2,5-dien-1-iminium (CASCADE BLUE™ dye), tris(N,N-diethylethanaminium) 8-[2-(4-{[(2,5-dioxopyrrolidin-1-yl)oxy]carbonyl}piperidin-1-yl)-2-oxoethoxy]pyrene-1,3,6-trisulfonate (ALEXA FLUOR 405™), N,N-diethylethanaminium [9-{6-[(2,5-dioxopyrrolidin-1-yl)oxy]-6-oxohexyl}-8,8-dimethyl-2-oxo-4-(trifluoromethyl)-8,9-dihydro-2H-benzo[g]chromen-6-yl]methanesulfonate (ALEXA FLUOR 430™) 1-[({4-[(7-nitro-2,1,3-benzoxadiazol-4-yl)amino]phenyl}acetyl)oxy]pyrrolidine-2,5-dione (QSY™ dye), fluorescein, 2-(6-amino-3-iminio-4,5-disulfonato-3H-xanthen-9-yl)-5-((2,3,5,6-tetrafluorophenoxy)carbonyl)benzoate (ALEXA FLUOR 488™), 2′,7′-Difluoro-3′,6′-dihydroxy-3H-spiro[isobenzofuran-1,9′-xanthen]-3-one (OREGON GREEN™ 488), 1,3,5,7,8-pentamethyl-4,4-difluorro-4-bora-3a,4a-diaza-s-indacene (BODIPY™493/503), rhodamine green, and rhodamine red.

103. The method of claim 102, wherein the compound is 2-[N-(7-nitrobenz-2-oxa-1,3-diazol-4-yl)amino]-2-deoxy-D-glucose.

104. The method of any one of claims 5, 6, and 9-103, wherein the rate of oxidative phosphorylation in the first and second samples of cells obtained from the subject prior to administration of BCG is evaluated by measuring the rate of oxygen consumption in the first and second samples of cells.

105. The method of any one of claims 7-104, wherein the rate of aerobic glycolysis in the first and second samples of cells obtained from the subject prior to administration of BCG is evaluated by measuring the rate of extracellular acidification in the first and second samples of cells.

106. The method of any one of claims 1-105, wherein the BCG is administered to the subject in a single dose.

107. The method of any one of claims 1-105, wherein the BCG is administered to the subject in multiple doses.

108. The method of claim 107, wherein the BCG is administered to the subject during a first and second, or subsequent, treatment period, wherein the treatment periods have a frequency of no greater than once per year, and wherein, during each treatment period, the BCG is administered to the subject in one or more doses, and the BCG is not administered to the subject during the time elapsed between the treatment periods.

109. The method of claim 108, wherein the BCG is administered to the subject in one or more doses per week, month, or year.

110. The method of claim 109, wherein the BCG is administered to the subject in from 1 to 10 doses per year.

111. The method of claim 110, wherein the BCG is administered to the subject in from 1 to 5 doses per year.

112. The method of claim 111, wherein the BCG is administered to the subject in 1 dose per year.

113. The method of claim 111, wherein the BCG is administered to the subject in 2 doses per year.

114. The method of claim 111, wherein the BCG is administered to the subject in 3 doses per year.

115. The method of claim 111, wherein the BCG is administered to the subject in 4 doses per year.

116. The method of claim 111, wherein the BCG is administered to the subject in 5 doses per year.

117. The method of any one of claims 107-116, wherein the BCG is administered to the subject in a total of from 2 to 20 doses.

118. The method of claim 117, wherein the BCG is administered to the subject in a total of from 2 to 10 doses.

119. The method of claim 118, wherein the BCG is administered to the subject in a total of from 2 to 5 doses.

120. The method of claim 119, wherein the BCG is administered to the subject in a total of 2 doses.

121. The method of claim 119, wherein the BCG is administered to the subject in a total of 3 doses.

122. The method of claim 119, wherein the BCG is administered to the subject in a total of 4 doses.

123. The method of claim 119, wherein the BCG is administered to the subject in a total of 5 doses.

124. The method of any one of claims 107-123, wherein a time elapsed from administration of one dose of the BCG to administration of a subsequent dose of the BCG is from about 1 day to about 1 year.

125. The method of claim 124, wherein the time between doses of the BCG is from about 1 week to about 12 weeks.

126. The method of claim 125, wherein the time between doses of the BCG is from about 2 weeks to about 8 weeks.

127. The method of claim 126, wherein the time between doses of the BCG is from about 3 weeks to about 6 weeks.

128. The method of claim 127, wherein the time between doses of the BCG is about 4 weeks.

129. The method of any one of claims 107-123, wherein a time elapsed from administration of one dose of the BCG to administration of a subsequent dose of the BCG is from about 2 months to about 72 months.

130. The method of claim 129, wherein the time between doses of the BCG is from 3 months to about 48 months.

131. The method of claim 130, wherein the time between doses of the BCG is from about 6 months to about 36 months.

132. The method of claim 131, wherein the time between doses of the BCG is from about 8 months to about 16 months.

133. The method of claim 132, wherein the time between doses of the BCG is about 12 months.

134. The method of any one of claims 1-105, wherein the BCG is administered to the subject in at least first, second, and third doses, wherein:
a) the second dose of the BCG is administered to the subject from about 1 day to about 1 year after administration of the first dose of the BCG to the subject; and
b) the third dose of the BCG is administered to the subject from about 2 months to about 72 months after administration of the second dose of the BCG to the subject.

135. The method of claim 134, wherein second dose of the BCG is administered to the subject from about 1 week to about 12 weeks after administration of the first dose of the BCG to the subject.

136. The method of claim 135, wherein the second dose of the BCG is administered to the subject from about 2 weeks to about 8 weeks after administration of the first dose of the BCG to the subject.

137. The method of claim 136, wherein the second dose of the BCG is administered to the subject from about 3 weeks to about 6 weeks after administration of the first dose of the BCG to the subject.

138. The method of claim 137, wherein the second dose of the BCG is administered to the subject about 4 weeks after administration of the first dose of the BCG to the subject.

139. The method of any one of claims 134-138, wherein the third dose of the BCG is administered to the subject from about 3 months to about 48 months after administration of the second dose of the BCG to the subject.

140. The method of claim 139, wherein the third dose of the BCG is administered to the subject from about 6 months to about 36 months after administration of the second dose of the BCG to the subject.

141. The method of claim 140, wherein the third dose of the BCG is administered to the subject from about 8 months to about 16 months after administration of the second dose of the BCG to the subject.

142. The method of claim 141, wherein the third dose of the BCG is administered to the subject about 12 months after administration of the second dose of the BCG to the subject.

143. The method of any one of claims 134-142, wherein the BCG is further administered to the subject in fourth, fifth, and sixth doses, wherein:
a) the fourth dose of the BCG is administered to the subject from about 2 months to about 72 months after administration of the third dose of the BCG to the subject;
b) the fifth dose of the BCG is administered to the subject from about 2 months to about 72 months after administration of the fourth dose of the BCG to the subject; and
c) the sixth dose of the BCG is administered to the subject from about 2 months to about 72 months after administration of the fifth dose of the BCG to the subject.

144. The method of claim 143, wherein the fourth dose of the BCG is administered to the subject from about 3 months to about 48 months after administration of the third dose of the BCG to the subject.

145. The method of claim 144, wherein the fourth dose of the BCG is administered to the subject from about 6 months to about 36 months after administration of the third dose of the BCG to the subject.

146. The method of claim 145, wherein the fourth dose of the BCG is administered to the subject from about 8 months to about 16 months after administration of the third dose of the BCG to the subject.

147. The method of claim 146, wherein the fourth dose of the BCG is administered to the subject about 12 months after administration of the third dose of the BCG to the subject.

148. The method of any one of claims 143-147, wherein the fifth dose of the BCG is administered to the subject from about 3 months to about 48 months after administration of the fourth dose of the BCG to the subject.

149. The method of claim 148, wherein the fifth dose of the BCG is administered to the subject from about 6 months to about 36 months after administration of the fourth dose of the BCG to the subject.

150. The method of claim 149, wherein the fifth dose of the BCG is administered to the subject from about 8 months to about 16 months after administration of the fourth dose of the BCG to the subject.

151. The method of claim 150, wherein the fifth dose of the BCG is administered to the subject about 12 months after administration of the fourth dose of the BCG to the subject.

152. The method of any one of claims 143-151, wherein the sixth dose of the BCG is administered to the subject from about 3 months to about 48 months after administration of the fifth dose of the BCG to the subject.

153. The method of claim 152, wherein the sixth dose of the BCG is administered to the subject from about 6 months to about 36 months after administration of the fifth dose of the BCG to the subject.

154. The method of claim 153, wherein the sixth dose of the BCG is administered to the subject from about 8 months to about 16 months after administration of the fifth dose of the BCG to the subject.

155. The method of claim 154, wherein the sixth dose of the BCG is administered to the subject about 12 months after administration of the fifth dose of the BCG to the subject.

156. The method of any one of claims 106-155, wherein each said dose comprises from about 1×10⁴ colony forming units (CFU) per 0.1 mg of BCG to about 1×10⁸ CFU per 0.1 mg of BCG.

157. The method of claim 156, wherein each said dose comprises from about 1×10⁵ CFU per 0.1 mg of BCG to about 1×10⁷ CFU per 0.1 mg of BCG.

158. The method of claim 157, wherein each said dose comprises from about 1×10⁶ CFU per 0.1 mg of BCG to about 9×10⁶ CFU per 0.1 mg of BCG.

159. The method of claim 158, wherein each said dose comprises from about 1.8×10⁶ CFU per 0.1 mg of BCG to about 3.9×10⁶ CFU per 0.1 mg of BCG.

160. The method of claim 159, wherein each said dose comprises about 1.8×10⁶ CFU per 0.1 mg of BCG, about 1.9×10⁶ CFU per 0.1 mg of BCG, about 2.0 CFU per 0.1 mg of BCG, about 2.1 CFU per 0.1 mg of BCG, about 2.2 CFU per 0.1 mg of BCG, about 2.3 CFU per 0.1 mg of BCG, about 2.4 CFU per 0.1 mg of BCG, about 2.5 CFU per 0.1 mg of BCG, about 2.6 CFU per 0.1 mg of BCG, about 2.7 CFU per 0.1 mg of BCG, about 2.8 CFU per 0.1 mg of BCG, about 2.9 CFU per 0.1 mg of BCG, about 3.0 CFU per 0.1 mg of BCG, about 3.1 CFU per 0.1 mg of BCG, about 3.2 CFU per 0.1 mg of BCG, about 3.3 CFU per 0.1 mg of BCG, about 3.4 CFU per 0.1 mg of BCG, about 3.5 CFU per 0.1 mg of BCG, about 3.6 CFU per 0.1 mg of BCG, about 3.7 CFU per 0.1 mg of BCG, or about 3.8 CFU per 0.1 mg of BCG.

161. The method of any one of claims 1-160, wherein the BCG is administered to the subject subcutaneously, intradermally, intravenously, or intraperitoneally.

162. The method of any one of claims 1-161, wherein, prior to administration of the BCG to the subject, the subject:
a) has a chronological age of from about 18 years to about 65 years;
b) has been living with the diabetes for a duration of from about 5 years to about 60 years;
c) has tested positive for presence of anti-GAD autoantibodies in peripheral blood;
d) has exhibited a fasting or stimulated c-peptide concentration of about 200 pM or less, optionally wherein the subject has a fasting or stimulated c-peptide concentration of from about 5 pM to about 200 pM;
e) has exhibited a blood glucose concentration of about 200 mg/dL or greater;
f) has exhibited an HbA1c level of 6.5% or greater; and/or
g) has exhibited a blood glucose concentration of about 100 mg/dL or greater in a fasting state, optionally wherein the fasting state is one in which the subject has not eaten for the previous 8-12 hours relative to the time at which the blood glucose concentration is measured.

163. The method of any one of claims 1-162, wherein, prior to being administered the BCG, the subject is undergoing insulin therapy, optionally wherein, prior to being administered the BCG, the subject is dependent upon the insulin therapy to maintain glucose homeostasis.

164. The method of claim 163, wherein the insulin therapy comprises administration of insulin to the subject in one or more doses per day, week, month, or year.

165. The method of claim 164, wherein the insulin therapy comprises administration of insulin to the subject in one or more doses per day.

166. The method of claim 164 or 165, wherein the insulin is administered to the subject subcutaneously, intravenously, parenterally, or by way of inhalation.

167. The method of any one of claims 164-166, wherein the insulin is administered to the subject in the form of an agent selected from insulin lispro injection (ADMELOG™), insulin glulisine (APIDRA™) insulin aspart (FIASP™), insulin lispro (HUMALOG™), human insulin isophane suspension (HUMULIN N™), insulin glargine (LANTUS™), insulin detemir (LEVEMIR™), insulin degludec (TRESIBA FLEXTOUCH™), 75% insulin lispro protamine suspension/25% insulin lispro injection (HUMALOG MIX 75/25™), 70% human insulin isophane suspension/30% human insulin injection (HUMALOG 70/30™) 50% insulin lispro protamine suspension/50% insulin lispro injection (HUMALOG MIX 50/50™), 70% insulin aspart protamine suspension/30% insulin aspart injection (NOVOLOG MIX 70/30™), 70% insulin degludec/30% insulin aspart (RYZODEG 70/30 FLEXTOUCH™), and 70% NPH human insulin/30% regular human insulin injection (HUMULIN 70/30™).

168. The method of any one of claims 163-167, wherein following administration of the BCG to the subject the frequency of the insulin therapy is reduced.

169. The method of any one of claims 163-168, wherein following administration of the BCG to the subject the quantity of insulin administered to the subject per dose is reduced.

170. The method of any one of claims 163-169, wherein following administration of the BCG to the subject the insulin therapy is ceased.

171. The method of any one of claims 1-170, wherein following administration of the BCG to the subject, the subject exhibits a reduction in HbA1c level of about 5% or more, optionally wherein following administration of the BCG to the subject, the subject exhibits a reduction in HbA1C level of from about 5% to about 15%.

172. The method of claim 171, wherein the subject exhibits the reduction in HbA1c level at least about 6 months following administration of the BCG to the subject, optionally wherein the subject exhibits the reduction in HbA1c level from about 6 months to about 36 months following administration of the BCG to the subject.

173. The method of any one of claims 1-172, wherein following administration of the BCG to the subject, the subject exhibits a blood glucose concentration of less than 200 mg/dL, optionally wherein the blood glucose concentration of less than 200 mg/dL is sustained without insulin therapy.

174. The method of claim 173, wherein the blood glucose concentration of less than 200 mg/dL is sustained in both a fed and fasting state.

175. The method of any one of claims 1-174, wherein following administration of the BCG to the subject, the subject exhibits an increase in aerobic glycolysis.

176. The method of any one of claims 1-175, wherein following administration of the BCG to the subject, the subject exhibits a reduction in oxidative phosphorylation.

177. The method of any one of claims 1-176, wherein the subject is a mammal.

178. The method of claim 177, wherein the mammal is a human.

179. The method of any one of claims 74-81 and 84-93, wherein the subject is a mammal.

180. The method of claim 179, wherein the subject is a human.

181. The method of any one of claims 1-180, wherein the diabetes is type 1 diabetes.

182. The method of any one of claims 1-180, wherein the diabetes is type 2 diabetes.

183. The method of any one of claims 1-182, wherein the subject has not been previously administered metformin.

184. The method of any one of claims 1-183, wherein the subject is not administered metformin following, or concurrently with, administration of BCG.

185. The method of any one of claims 1-184, wherein the subject does not have latent autoimmune diabetes in adults (LADA).

186. The method of any one of claims 1-185, wherein the subject does not have or has not previously had bladder cancer.

187. The method of any one of claims 1-186, wherein the subject is 40 years old or more at the time of being administered the BCG (e.g., 40, 41, 42, 43, 44, 45, 46, 47, 48, 49, 50, 51, 52, 53, 54, 55, 56, 57, 58, 59, 60, 61, 62, 63, 64, or 65 years old, or more, at the time of being administered the BCG).

188. The method of any one of claims 1, 2, and 9-187, wherein the reference rate of glucose uptake is about 6,000 mean fluorescence intensity (MFI) units to about 10,000 MFI units, as assessed by subjecting a cell of the subject to a fluorescently-labeled glucose uptake assay.

189. The method of any one of claims 1, 2, and 9-188, wherein the reference rate of oxidative phosphorylation is from about 25 pmol of O₂ consumption per minute to about 150 pmol of O₂ consumption per minute.

190. The method of any one of claims 1, 2, and 9-189, wherein the reference rate of aerobic glycolysis is from about 5 to about 50 mpH units per minute.

191. A kit comprising: (i) one or more unit dosage forms of BCG and (ii) a package insert, wherein the package insert instructs a user of the kit to administer the BCG to a subject having diabetes in accordance with the method of any one of claims 1-190.

192. The kit of claim 191, wherein the BCG is formulated for subcutaneous, intradermal, intravenous, or intraperitoneal injection to the subject.

193. The kit of claim 191 or 192, wherein each unit dosage form comprises from about 1×10⁴ colony forming units (CFU) per 0.1 mg of BCG to about 1×10⁸ CFU per 0.1 mg of BCG.

194. The kit of claim 193, wherein each unit dosage form comprises from about 1×10⁵ CFU per 0.1 mg of BCG to about 1×10⁷ CFU per 0.1 mg of BCG.

195. The kit of claim 194, wherein each unit dosage form comprises from about 1×10⁶ CFU per 0.1 mg of BCG to about 9×10⁶ CFU per 0.1 mg of BCG.

196. The kit of claim 195, wherein each unit dosage form comprises from about 1.8×10⁶ CFU per 0.1 mg of BCG to about 3.9×10⁶ CFU per 0.1 mg of BCG.

197. The kit of claim 196, wherein each unit dosage form comprises about 1.8×10⁶ CFU per 0.1 mg of BCG, about 1.9×10⁶ CFU per 0.1 mg of BCG, about 2.0 CFU per 0.1 mg of BCG, about 2.1 CFU per 0.1 mg of BCG, about 2.2 CFU per 0.1 mg of BCG, about 2.3 CFU per 0.1 mg of BCG, about 2.4 CFU per 0.1 mg of BCG, about 2.5 CFU per 0.1 mg of BCG, about 2.6 CFU per 0.1 mg of BCG, about 2.7 CFU per 0.1 mg of BCG, about 2.8 CFU per 0.1 mg of BCG, about 2.9 CFU per 0.1 mg of BCG, about 3.0 CFU per 0.1 mg of BCG, about 3.1 CFU per 0.1 mg of BCG, about 3.2 CFU per 0.1 mg of BCG, about 3.3 CFU per 0.1 mg of BCG, about 3.4 CFU per 0.1 mg of BCG, about 3.5 CFU per 0.1 mg of BCG, about 3.6 CFU per 0.1 mg of BCG, about 3.7 CFU per 0.1 mg of BCG, or about 3.8 CFU per 0.1 mg of BCG.

198. A kit comprising: (i) a compound comprising glucose covalently bound to a fluorophore and (ii) a package insert, wherein the package insert instructs a user of the kit to select a subject having diabetes for treatment with BCG in accordance with the method of any one of claims 2, 4, and 9-190.

199. The kit of claim 198, wherein the fluorophore comprises a moiety selected from 7-nitrobenz-2-oxa-1,3-diazol-4-yl (NBD), methoxycoumarin, dialkylaminocoumarin, hydroxycoumarin, aminomethylcoumarin acetate (AMCA), 6,8-difluoro-7-hydroxy-3-carboxycoumarin (PACIFIC BLUE™ dye), 5-(dimethylamino)naphthalene-1-sulfonyl (Dansyl), pyrene, 7-amino-3-{[(2,5-dioxopyrrolidin-1-yl)oxy]-2-oxoethyl}-4-methyl-2-oxo-2H-chromene-6-sulfonic acid (ALEXA FLUOR 350™), 6,8-difluoro-7-hydroxy-4-methylcoumarin (MARINA BLUE™ dye), N-(2-aminoethyl)-4-{5-[4-(dimethylamino)phenyl]-1,3-oxazol-2-yl}benzenesulfonamide (DAPOXYL™ dye), 2,3,5,6-Tetramethyl-1H,7H-pyrazolo[1,2-a]pyrazole-1,7-dione (Bimane), 4-{[4-(Diethylamino)phenyl][4-(ethylamino)naphthalen-2-yl]methylidene}-N,N-diethylcyclohexa-2,5-dien-1-iminium (CASCADE BLUE™ dye), tris(N,N-diethylethanaminium) 8-[2-(4-{[(2,5-dioxopyrrolidin-1-yl)oxy]carbonyl}piperidin-1-yl)-2-oxoethoxy]pyrene-1,3,6-trisulfonate (ALEXA FLUOR 405™), N,N-diethylethanaminium [9-{6-[(2,5-dioxopyrrolidin-1-yl)oxy]-6-oxohexyl}-8,8-dimethyl-2-oxo-4-(trifluoromethyl)-8,9-dihydro-2H-benzo[g]chromen-6-yl]methanesulfonate (ALEXA FLUOR 430™) 1-[({4-[(7-nitro-2,1,3-benzoxadiazol-4-yl)amino]phenyl}acetyl)oxy]pyrrolidine-2,5-dione (QSY™ dye), fluorescein, 2-(6-amino-3-iminio-4,5-disulfonato-3H-xanthen-9-yl)-5-((2,3,5,6-tetrafluorophenoxy)carbonyl)benzoate (ALEXA FLUOR 488™), 2′,7′-Difluoro-3′,6′-dihydroxy-3H-spiro[isobenzofuran-1,9′-xanthen]-3-one (OREGON GREEN™ 488), 1,3,5,7,8-pentamethyl-4,4-difluorro-4-bora-3a,4a-diaza-s-indacene (BODIPY™493/503), rhodamine green, and rhodamine red.

200. The kit of claim 199, wherein the compound is 2-[N-(7-nitrobenz-2-oxa-1,3-diazol-4-yl)amino]-2-deoxy-D-glucose.

201. A kit comprising: (i) one or more agents capable of detecting oxygen consumption in a cell and (ii) a package insert, wherein the package insert instructs a user of the kit to select a subject having diabetes for treatment with BCG in accordance with the method of any one of claims 5, 6, and 9-190.

202. The kit of claim 201, wherein the kit further comprises an electron transport chain inhibitor.

203. The kit of claim 202, wherein the electron transport chain inhibitor is rotenone or antimycin A.

204. A kit comprising: (i) one or more agents capable of detecting extracellular pH in a sample of cells and (ii) a package insert, wherein the package insert instructs a user of the kit to select a subject having diabetes for treatment with BCG in accordance with the method of any one of claims 7-190.

205. The kit of any one of claims 198-204, wherein the kit further comprises one or more unit dosage forms of BCG, optionally wherein the package insert further instructs a user of the kit to administer BCG to the subject identified as likely to respond to the BCG.

206. The kit of claim 205, wherein the BCG is formulated for subcutaneous, intradermal, intravenous, or intraperitoneal injection to the subject.

207. The kit of claim 205 or 206, wherein each unit dosage form comprises from about 1×10⁴ colony forming units (CFU) per 0.1 mg of BCG to about 1×10⁸ CFU per 0.1 mg of BCG.

208. The kit of claim 207, wherein each unit dosage form comprises from about 1×10⁵ CFU per 0.1 mg of BCG to about 1×10⁷ CFU per 0.1 mg of BCG.

209. The kit of claim 208, wherein each unit dosage form comprises from about 1×10⁶ CFU per 0.1 mg of BCG to about 9×10⁶ CFU per 0.1 mg of BCG.

210. The kit of claim 209, wherein each unit dosage form comprises from about 1.8×10⁶ CFU per 0.1 mg of BCG to about 3.9×10⁶ CFU per 0.1 mg of BCG.

211. The kit of claim 210, wherein each unit dosage form comprises about 1.8×10⁶ CFU per 0.1 mg of BCG, about 1.9×10⁶ CFU per 0.1 mg of BCG, about 2.0 CFU per 0.1 mg of BCG, about 2.1 CFU per 0.1 mg of BCG, about 2.2 CFU per 0.1 mg of BCG, about 2.3 CFU per 0.1 mg of BCG, about 2.4 CFU per 0.1 mg of BCG, about 2.5 CFU per 0.1 mg of BCG, about 2.6 CFU per 0.1 mg of BCG, about 2.7 CFU per 0.1 mg of BCG, about 2.8 CFU per 0.1 mg of BCG, about 2.9 CFU per 0.1 mg of BCG, about 3.0 CFU per 0.1 mg of BCG, about 3.1 CFU per 0.1 mg of BCG, about 3.2 CFU per 0.1 mg of BCG, about 3.3 CFU per 0.1 mg of BCG, about 3.4 CFU per 0.1 mg of BCG, about 3.5 CFU per 0.1 mg of BCG, about 3.6 CFU per 0.1 mg of BCG, about 3.7 CFU per 0.1 mg of BCG, or about 3.8 CFU per 0.1 mg of BCG.
